(19) United States
(12) Patent Application Publication
(10) Pub. No.: US 2026/0315083 A1
(43) Pub. Date: Oct. 15, 2026

(54) ROBOTICALLY CONTROLLED MULTI-DEVICE STACK FOR TREATMENT OF PULMONARY EMBOLISM AND METHODS OF USE

(71) Applicant: Imperative Care, Inc., Campbell, CA (US)

(72) Inventor: Theodore August Jourdan, Mt. Pleasant, SC (US)

(21) Appl. No.: 19/630,953

(22) Filed: Mar. 27, 2026

Related U.S. Application Data
(60) Provisional application No. 63/780,089, filed on Mar. 28, 2025.

(51) Int. Cl. A61B 17/22; A61B 17/00; A61B 34/30; A61M 25/00; A61M 25/01; A61M 25/09

(52) U.S. Cl. CPC A61B 17/22; A61B 34/30; A61B 2017/003; A61B 2017/00876; A61B 2017/22038; A61B 2017/22079; A61B 2017/22084; A61B 2034/301; A61B 2034/303; A61M 25/0074; A61M 25/0113; A61M 25/09041; A61M 2025/091

(57) ABSTRACT

A multi-device stack can include a sheath, a procedure catheter, an insert catheter, and a guidewire. The multi-device stack can be utilized according to a method of operation to treat pulmonary embolism. An insertion kit can include a puncture needle, a micro wire, and a dilator. The insertion kit can be utilized according to an insertion method to introduce the multi-device stack into vasculature of a patient. The multi-device stack can be robotically controlled by a robotic drive system.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57. This application claims priority benefit of U.S. Provisional Application No. 63/780,089 filed Mar. 28, 2025, which is hereby incorporated by reference in its entirety herein.

### BACKGROUND

### Field

The present application relates to neurovascular procedures, and more particularly, to catheter assemblies and robotic control systems for neurovascular site access.

### Description of the Related Art

A variety of neurovascular procedures can be accomplished via a transvascular access, including thrombectomy, diagnostic angiography, embolic coil deployment and stent placement. However, the delivery of neurovascular care is limited or delayed by a variety of challenges. For example, there are not enough trained interventionalists and centers to meet the current demand for neuro interventions. Neuro interventions are difficult, with complex set up requirements and demands on the surgeon's dexterity. With two hands, the surgeon must exert precise control over 3-4 coaxial catheters plus manage the fluoroscopy system and patient position. Long, tortuous anatomy, requires delicate, precise maneuvers. Inadvertent catheter motion can occur due to energy storage and release caused by frictional interplay between coaxial shafts and the patient's vasculature. Supra-aortic access necessary to reach the neurovasculature is challenging to achieve, especially Type III arches. Once supra-aortic access is achieved, adapting the system for neurovascular treatments is time consuming and requires guidewire and access catheter removal and addition of a procedure catheter (and possibly one or more additional catheters) to the stack.

Thus, there remains a need for a supra-aortic access and neurovascular site access system that addresses some or all these challenges and increases the availability of neurovascular procedures. Preferably, the system is additionally capable of driving devices further distally through the supra-aortic access to accomplish procedures in the intracranial vessels.

### SUMMARY

The present disclosure provides a system for treating a pulmonary embolism. The system includes a coaxial multi-device assembly including a sheath coupled to a sheath hub, wherein the sheath hub is configured to adjust an axial position of the sheath, a procedure catheter positioned within the sheath and coupled to a procedure catheter hub, wherein the procedure catheter hub is configured to adjust an axial position of the procedure catheter, and an access device positioned within the procedure catheter and coupled to an access device hub, wherein the access device hub is configured to adjust an axial position and a rotational position of the access device, wherein the access device includes a tip configured to transition between a curled configuration and a straight configuration, and a robotic drive system configured to drive axial movement of the sheath hub, the procedure catheter hub, and the access device hub.

In some embodiments, the coaxial multi-device assembly further includes a guidewire positioned within the access device and coupled to a guidewire hub, wherein the guidewire hub is configured to drive axial and rotational movement of the guidewire. In some embodiments, the access device is in fluid communication with a contrast source and configured to inject contrast while the guidewire is positioned within the access device. In some embodiments, the guidewire includes a hollow guidewire. In some embodiments, the guidewire is in fluid communication with a source of a drug to provide the drug through the guidewire. In some embodiments, the drug includes a tissue plasminogen activator. In some embodiments, the guidewire is in fluid communication with an aspiration source to provide aspiration through the guidewire. In some embodiments, the guidewire includes a tip configured to transition between a curled configuration and a straight configuration. In some embodiments, the access device includes a tapered distal section. In some embodiments, the robotic drive system includes: a sheath hub adapter configured to couple to the sheath hub to drive axial movement of the sheath hub, a procedure catheter hub adapter configured to couple to the procedure catheter hub to drive axial movement of the procedure catheter, and an access device hub adapter configured to couple to the access device hub to drive axial movement of the access device. In some embodiments, the sheath hub adapter is magnetically coupled to the sheath hub, wherein the procedure catheter hub adapter is magnetically coupled to the procedure catheter hub, and wherein the access device hub adapter is magnetically coupled to the access device hub. In some embodiments, the access device is an access catheter.

The present disclosure also provides a method of treating a pulmonary embolism. The method includes introducing a multi-device assembly into a blood vessel of a patient, the multi-device assembly including a sheath, a procedure catheter, and an access device, advancing the multi-device assembly through the inferior vena cava and into the right atrium of the patient, advancing the access device through the right atrium of the patient, through the right ventricle of the patient, and into the pulmonary trunk of the patient, advancing the access device into a pulmonary branch and to a treatment site, advancing the procedure catheter over the access device and to the treatment site, removing the access device, and performing a thrombectomy using the procedure catheter. In some embodiments, the method further includes advancing the sheath over the procedure catheter and the access device into the pulmonary trunk. In some embodiments, the step of advancing the sheath over the procedure catheter and the access device into the pulmonary trunk is performed after advancing the access device into the pulmonary trunk of the patient and before advancing the access device into the pulmonary branch. In some embodiments, the multi-device assembly further includes a guidewire. In some embodiments, the step of advancing the access device through the right atrium of the patient, through the right ventricle of the patient, and into the pulmonary trunk of the patient includes advancing the access device and the guidewire through the right atrium of the patient, through the right ventricle of the patient, and into the pulmonary trunk of the patient, wherein the step of advancing the access device into the pulmonary branch and to the treatment site includes advancing the access device and the guidewire into the pulmonary branch and to the treatment site, and wherein the step of removing the access device includes removing the access device and the guidewire. In some embodiments, the method further includes injecting contrast through the access device to identify the treatment site while the guidewire is positioned within the access device. In some embodiments, the method further includes, after performing the thrombectomy with the procedure catheter, withdrawing the sheath and the procedure catheter from the pulmonary branch and advancing the sheath and procedure catheter into a different pulmonary branch without reinserting the access device and the guidewire. In some embodiments, the guidewire includes a hollow guidewire. In some embodiments, the method further includes delivering a drug through the guidewire. In some embodiments, the drug includes a tissue plasminogen activator. In some embodiments, the method further includes aspirating through the guidewire to anchor a blood clot to the guidewire. In some embodiments, the guidewire includes a tip configured to transition between a curled configuration and a straight configuration. In some embodiments, advancing the guidewire through the right atrium of the patient, through the right ventricle of the patient, and into the pulmonary trunk of the patient is performed while the tip of the guidewire is in the curled configuration. In some embodiments, the method further includes transitioning the tip of the guidewire from the curled configuration to the straight configuration within the pulmonary trunk. In some embodiments, the access device includes a tip configured to transition between a curled configuration and a straight configuration. In some embodiments, advancing the access device through the right atrium of the patient, through the right ventricle of the patient, and into the pulmonary trunk of the patient is performed while the tip of the access device is in the curled configuration. In some embodiments, the method further includes transitioning the tip of the access device from the curled configuration to the straight configuration within the pulmonary trunk. In some embodiments, the access device includes a tapered distal section. In some embodiments, the method further includes coupling the multi-device assembly to a robotic drive system, wherein at least the steps of advancing the multi-device assembly through the inferior vena cava and into the right atrium of the patient, advancing the access device through the right atrium of the patient, through the right ventricle of the patient, and into the pulmonary trunk of the patient, advancing the access device into the pulmonary branch and to the treatment site, and advancing the procedure catheter over the access device to the treatment site are performed by the robotic drive system. In some embodiments, the sheath is coupled to a sheath hub, the procedure catheter is coupled to a procedure catheter hub, and the access device is coupled to an access device hub. In some embodiments, coupling the multi-device assembly to the robotic drive system includes coupling the sheath hub to a sheath hub adapter, coupling the procedure catheter hub to a procedure catheter hub adapter, and coupling the access device hub to an access device hub adapter. In some embodiments, driving the multi-device assembly through the inferior vena cava and into the right atrium of the patient includes driving the multi-device assembly in response to movement of each of the sheath hub adapter, the procedure catheter hub adapter, and the access device hub adapter. In some embodiments, the sheath hub is magnetically coupled to the sheath hub adapter, the procedure catheter hub is magnetically coupled to the procedure catheter hub adapter, and the access device hub is magnetically coupled to the access device hub adapter. In some embodiments, the access device is an insert catheter.

The present disclosure also provides a method of treating a pulmonary embolism. The method includes introducing a multi-device assembly into a blood vessel of a patient, the multi-device assembly including a plurality of interventional devices co-axially movably assembled into the multi-device assembly, advancing the multi-device assembly through the inferior vena cava and into the right atrium of the patient, advancing a first subset of the plurality of interventional devices of the multi-device assembly through the right atrium of the patient, through the right ventricle of the patient, and into the pulmonary trunk of the patient, advancing the first subset into a pulmonary branch and to a treatment site, advancing a second subset of the plurality of interventional devices over the first subset and to the treatment site, advancing a third subset of the plurality of interventional devices of the multi-device assembly over the first subset and the second subset and into the pulmonary trunk, removing the first subset, and performing a thrombectomy procedure.

In some embodiments, the first subset includes an access device and a guidewire. In some embodiments, the second subset includes a procedure catheter. In some embodiments, the third subset includes a sheath. In some embodiments, the method further includes coupling the multi-device assembly to a robotic drive system, wherein at least the steps of advancing the multi-device assembly through the inferior vena cava and into the right atrium of the patient, advancing the first subset of the plurality of interventional devices of the multi-device assembly through the right atrium of the patient, through the right ventricle of the patient, and into the pulmonary trunk of the patient, advancing the first subset into the pulmonary branch and to the treatment site, advancing the second subset of the plurality of interventional devices over the first subset and to the treatment site, and advancing the third subset of the plurality of interventional devices of the multi-device assembly over the first subset and the second subset and into the pulmonary trunk are performed by the robotic drive system. In some embodiments, the step of advancing the third subset of the plurality of interventional devices of the multi-device assembly over the first subset and the second subset and into the pulmonary trunk is performed after advancing the first subset into the pulmonary trunk and before advancing the first subset into the pulmonary branch.

The present disclosure also provides an access device. The access device includes an obturator portion including a tapered distal end, a first bend located in the obturator portion, a second bend located distal to the first bend, and a pigtail portion configured to extend from the tapered distal end of the obturator portion.

In some embodiments, the first bend is between 20 degrees to 60 degrees, and the second bend is between 20 degrees to 60 degrees. In some embodiments, the first bend is 45 degrees, and the second bend is 45 degrees. In some embodiments, the first bend and the second bend are oriented in a same plane. In some embodiments, the pigtail portion is curled within the same plane as the first bend and the second bend. In some embodiments, the second bend is oriented in a different plane from the first bend. In some embodiments, the first bend is within a first plane and the second bend is within a second plane, wherein the second plane is oriented at between 45 degrees to 315 degrees relative to the first plane. In some embodiments, the second plane is oriented at 270 degrees relative to the first plane. In some embodiments, the pigtail portion is curled within the second plane. In some embodiments, the first bend and the second bend are oriented in a same direction, wherein the same direction is either a clockwise direction or a counterclockwise direction. In some embodiments, the pigtail portion curls in an opposite direction to the first bend and the second bend. In some embodiments, the first bend is positioned between 2 cm to 5 cm away from the second bend. In some embodiments, the pigtail portion is fixedly coupled to the tapered distal end of the obturator portion. In some embodiments, the obturator portion includes a lumen, and wherein the pigtail portion is configured to slidably move within the lumen between a first configuration in which the pigtail portion is retracted within the lumen and a second configuration in which the pigtail portion at least partially extends out of the tapered distal end of the obturator portion. In some embodiments, the pigtail portion includes a lumen and a side port in fluid communication with the lumen. In some embodiments, the access device further includes a stiffening member configured to be advanced through the access device to selectively straighten the first bend, the second bend, and the pigtail portion. In some embodiments, the access device includes an access dilator.

The present disclosure also provides a multi-device stack. The multi-device stack includes a sheath, a procedure catheter positioned coaxially within the sheath, and the access device positioned coaxially within the procedure catheter.

In some embodiments, the access device includes an obturator portion, a first bend located in the obturator portion, a second bend located distal to the first bend, and a pigtail portion configured to extend from a distal end of the obturator portion, wherein the pigtail portion curls in an opposite direction to the first bend and the second bend. In some embodiments, the first bend is between 20 degrees to 60 degrees, and the second bend is between 20 degrees to 60 degrees. In some embodiments, the access device is an access dilator.

The present disclosure also provides a method of treating a pulmonary embolism in a patient. The method includes introducing an access device into vasculature of the patient, wherein the access device includes an obturator portion, a first bend located in the obturator portion, a second bend located distal to the first bend, and a pigtail portion configured to extend from a distal end of the obturator portion, advancing the access device through a heart and a lung of the patient until the pigtail portion is adjacent to a treatment site within a pulmonary artery of the patient, wherein the treatment site is adjacent to the pulmonary embolism, advancing an aspiration catheter to the treatment site via the access device, retracting the access device from the treatment site, and applying aspiration via the aspiration catheter to aspirate and remove at least a portion of the pulmonary embolism.

### BRIEF DESCRIPTION OF THE DRAWINGS

FIG. 1 is a schematic perspective view of an interventional setup having an imaging system, a patient support table, and a robotic drive system in accordance with the present disclosure.

FIG. 2 is a longitudinal cross section showing the concentric relationship between a guidewire having two degrees of freedom, an access catheter having 3 degrees of freedom and a guide catheter having one degree of freedom.

FIG. 3A is an exploded schematic view of interventional device hubs separated from a support table by a sterile barrier.

FIGS. 3B-3F show an alternate sterile barrier in the form of a shipping tray having one or more storage channels for carrying interventional devices.

FIGS. 3G-3K show embodiments of an alternate sterile barrier having a convex drive surface.

FIGS. 3L and 3M depict an example of a hub that may be used with the sterile barriers of FIGS. 3G-3K.

FIG. 4 is a schematic elevational cross section through a hub adapter having a drive magnet separated from an interventional device hub and driven magnet by a sterile barrier.

FIGS. 5A and 5B schematically illustrate a three interventional device and a four interventional device assembly.

FIG. 6 is a perspective view of a hub assembly.

FIG. 7 illustrates a clot capture and visualization device that can be integrated into a hub and/or connected to an aspiration line.

FIG. 8 illustrates a side elevational schematic view of an interventional device assembly for supra-aortic access and neuro-interventional procedures.

FIGS. 9A-9E depict an example sequence of steps of introducing a catheter assembly configured to achieve supra-aortic access and neurovascular site access.

FIG. 10 illustrates an example of a telescoping drive table.

FIG. 11 illustrates a system diagram of an embodiment of a medical device operation system.

FIG. 12A depicts a schematic view of an example multi-catheter stack having an access sheath, an aspiration catheter, an insert catheter, and a guidewire.

FIG. 12B depicts a schematic view of the multi-catheter stack of FIG. 12A with the insert catheter in a straightened configuration.

FIG. 12C depicts an enlarged region of the multi-catheter stack shown in FIG. 12B.

FIG. 13A depicts an enlarged schematic view of the insert catheter of FIG. 12A in a curled configuration.

FIG. 13B depicts an enlarged schematic view of the insert catheter of FIG. 12A in a straightened configuration.

FIG. 14A depicts a schematic view of the insert catheter of FIG. 12A being traversed through a heart.

FIG. 14B depicts a schematic view of the insert catheter of FIG. 12A being traversed through the lungs.

FIG. 15A depicts an enlarged schematic view of the guidewire of FIG. 12A in a curled configuration.

FIG. 15B depicts an enlarged schematic view of the guidewire of FIG. 12A in a straightened configuration.

FIG. 16 depicts a schematic view of the multi-catheter stack of FIG. 12A at a treatment site within a blood vessel.

FIGS. 17A-17U depict steps and sub-steps of an example method of operating the multi-catheter stack of FIG. 12A for treatment of a blood clot, such as a pulmonary embolism.

FIGS. 18A-18J depict steps of an example method for inserting the multi-catheter stack of FIG. 12A into the vasculature of a patient.

FIG. 19 depicts an example interventional setup for utilizing the multi-catheter stack of FIG. 12A.

FIG. 20A depicts examples of distal ends of insert catheters.

FIG. 20B depicts an example of a distal end of an insert catheter.

FIG. 20C depicts an example of a distal end of an insert catheter.

FIG. 20D depicts an example of a distal end of an insert catheter.

FIG. 21 is a side view of an embodiment of an access dilator.

FIG. 22A is a side view of another embodiment of an access dilator, showing a pigtail portion in a retracted or removed configuration.

FIG. 22B is a side view of the access dilator of FIG. 22A, showing the pigtail portion in a deployed configuration.

FIG. 23A is a schematic top view of another embodiment of an access dilator having a first bend and a second bend in different planes.

FIG. 23B is a schematic front view of the access dilator of FIG. 23A.

FIG. 23C is a schematic side view of the access dilator of FIG. 23A.

FIG. 24A is a schematic view of a multi-device stack with the access dilator of FIG. 22A, showing the access dilator in a deployed configuration.

FIG. 24B is a schematic view of the multi-device stack of FIG. 24A, showing the access dilator in a straightened configuration.

### DETAILED DESCRIPTION

In certain embodiments, a system is provided for advancing a guide catheter from a femoral artery or radial artery access into the ostium of one of the great vessels at the top of the aortic arch, thereby achieving supra-aortic access. A surgeon can then take over and advance interventional devices into the cerebral vasculature via the robotically placed guide catheter.

In some implementations, the system may additionally be configured to robotically gain intra-cranial vascular access and to perform an aspiration thrombectomy or other neuro vascular procedure.

A drive table can be positioned over or alongside the patient, and configured to axially advance, retract, and in some cases rotate and/or laterally deflect two or three or more different (e.g., concentrically or side by side oriented) intravascular devices. The hub is moveable along a path along the surface of the drive table to advance or retract the interventional device as desired. Each hub may also contain mechanisms to rotate or deflect the device as desired, and is connected to fluid delivery tubes (not shown) of the type conventionally attached to a catheter hub. Each hub can be in electrical communication with an electronic control system, either via hard wired connection, RF wireless connection or a combination of both.

Each hub is independently movable across the surface of a sterile field barrier membrane carried by the drive table. Each hub is releasably magnetically coupled to a unique drive carriage on the table side of the sterile field barrier. The drive system independently moves each hub in a proximal or distal direction across the surface of the barrier, to move the corresponding interventional device proximally or distally within the patient's vasculature.

The carriages on the drive table, which magnetically couple with the hubs to provide linear motion actuation, are universal. Functionality of the catheters/guidewire are provided based on what is contained in the hub and the shaft designs. This allows flexibility to configure the system to do a wide range of procedures using a wide variety of interventional devices on the same drive table. Additionally, the interventional devices and methods disclosed herein can be readily adapted for use with any of a wide variety of other drive systems (e.g., any of a wide variety of robotic surgery drive systems).

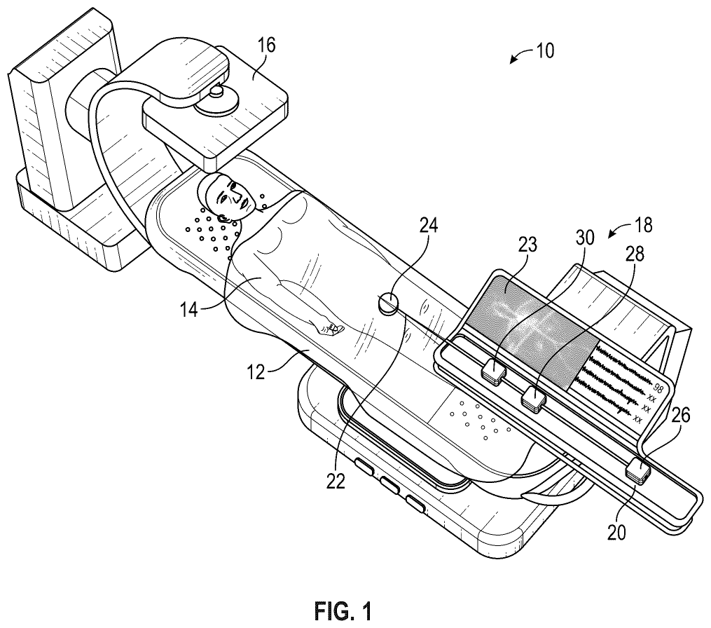

FIG. 1 is a schematic perspective view of an interventional setup 10 having a patient support table 12 for supporting a patient 14. An imaging system 16 may be provided, along with a robotic interventional device drive system 18 in accordance with the present disclosure.

The drive system 18 may include a support table 20 for supporting, for example, a guidewire hub 26, an access catheter hub 28 and a guide catheter hub 30. In the present context, the term “access” catheter can be any catheter having a lumen with at least one distally facing or laterally facing distal opening, that may be utilized to aspirate thrombus, provide access for an additional device to be advanced therethrough or therealong, or to inject saline or contrast media or therapeutic agents.

More or fewer interventional device hubs may be provided depending upon the desired clinical procedure. For example, in certain embodiments, a diagnostic angiogram procedure may be performed using only a guidewire hub 26 and an access catheter hub 28 for driving a guidewire and an access catheter (in the form of a diagnostic angiographic catheter), respectively. Multiple interventional devices 22 extend between the support table 20 and (in the illustrated example) a femoral access point 24 on the patient 14. Depending upon the desired procedure, access may be achieved by percutaneous or cut down access to any of a variety of arteries or veins, such as the femoral artery or radial artery. Although disclosed herein primarily in the context of neuro vascular access and procedures, the robotic drive system and associated interventional devices can readily be configured for use in a wide variety of additional medical interventions, in the peripheral and coronary arterial and venous vasculature, gastrointestinal system, lymphatic system, cerebral spinal fluid lumens or spaces (such as the spinal canal, ventricles, and subarachnoid space), pulmonary airways, treatment sites reached via trans ureteral or urethral or fallopian tube navigation, or other hollow organs or structures in the body (for example, in intra-cardiac or structural heart applications, such as valve repair or replacement, or in any endoluminal procedures).

A display 23, such as for viewing fluoroscopic images, catheter data (e.g., fiber Bragg grating fiber optics sensor data or other force or shape sensing data) or other patient data may be carried by the support table 20 and/or patient support table 12. Alternatively, the physician input/output interface including display 23 may be remote from the patient, such as behind radiation shielding, in a different room from the patient, or in a different facility than the patient.

In the illustrated example, a guidewire hub 26 is carried by the support table 20 and is moveable along the table to advance a guidewire into and out of the patient 14. An access catheter hub 28 is also carried by the support table 20 and is movable along the table to advance the access catheter into and out of the patient 14. The access catheter hub may also be configured to rotate the access catheter in response to manipulation of a rotation control, and may also be configured to laterally deflect a deflectable portion of the access catheter, in response to manipulation of a deflection control.

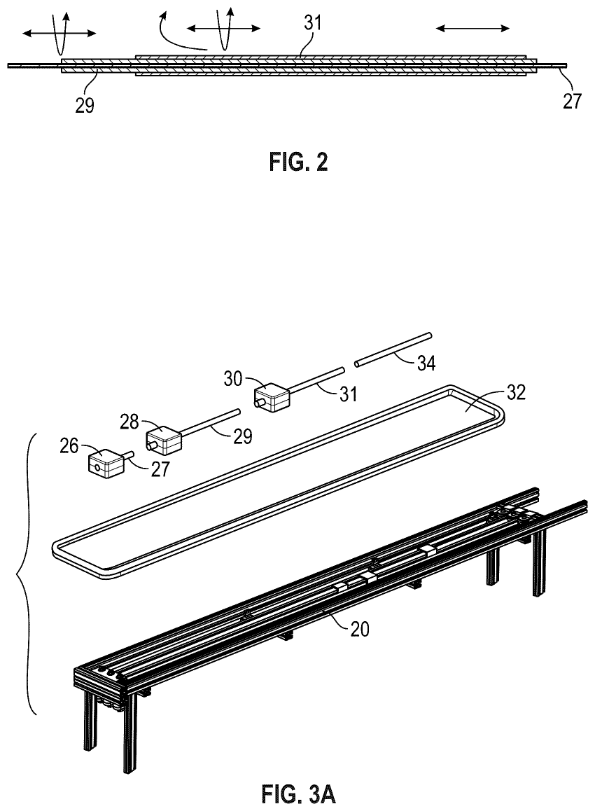

FIG. 2 is a longitudinal cross section schematically showing the motion relationship between a guidewire 27 having two degrees of freedom (axial and rotation), an access catheter 29 having three degrees of freedom (axial, rotational and lateral deflection) and a guide catheter 31, having one degree of freedom (axial).

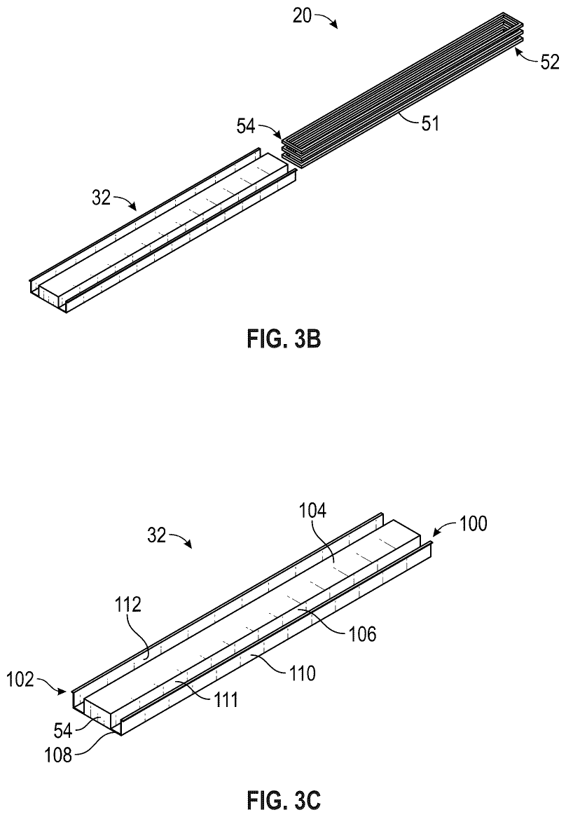

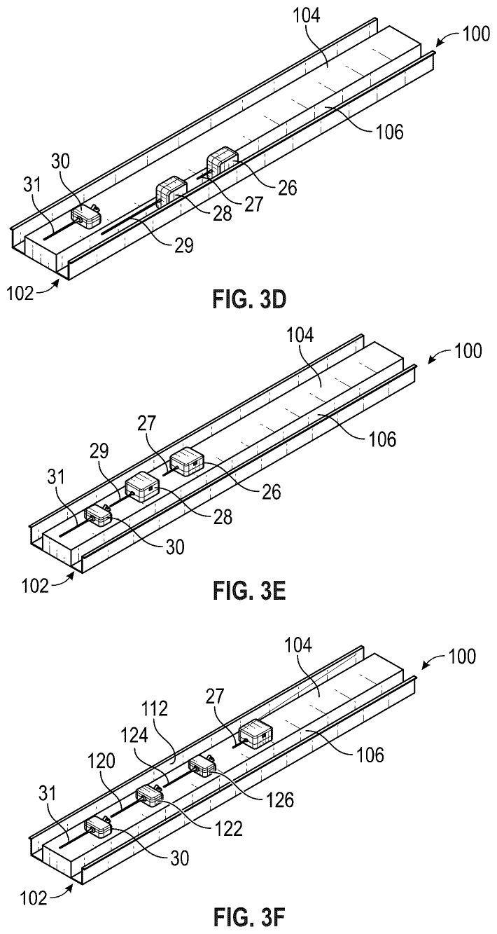

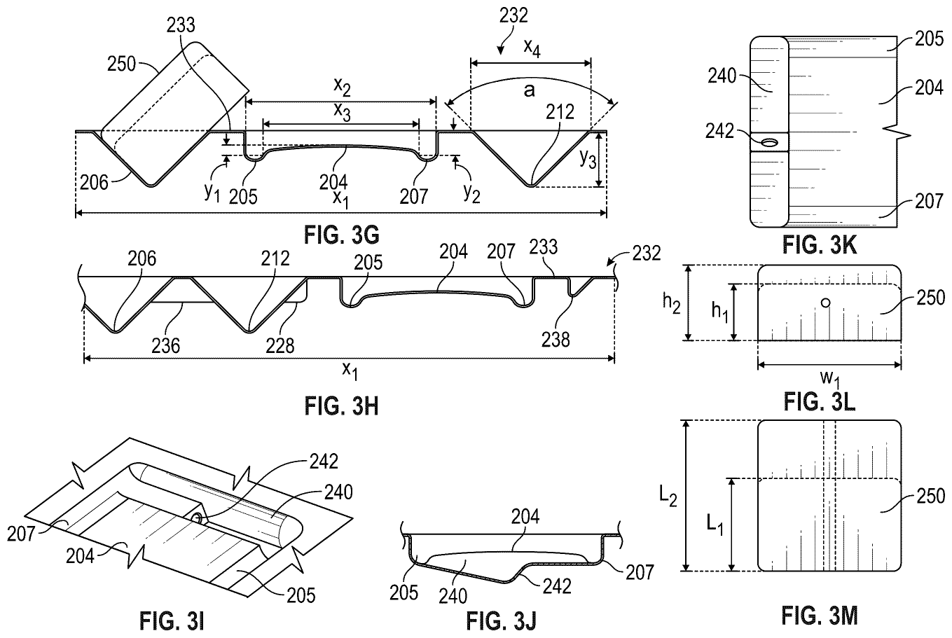

Referring to FIG. 3A, the support table 20 includes a drive mechanism described in greater detail below, to independently drive the guidewire hub 26, access catheter hub 28, and guide catheter hub 30. An anti-buckling feature 34 may be provided in a proximal anti-buckling zone for resisting buckling of the portion of the interventional devices spanning the distance between the support table 20 and the femoral artery access point 24. The anti-buckling feature 34 may include a plurality of concentric telescopically axially extendable and collapsible tubes through which the interventional devices extend.

Alternatively, a proximal segment of one or more of the device shafts may be configured with enhanced stiffness to reduce buckling under compression. For example, a proximal reinforced segment may extend distally from the hub through a distance of at least about 5 centimeters or 10 centimeters but typically no more than about 120 centimeters or 100 centimeters to support the device between the hub and the access point 24 on the patient. Reinforcement may be accomplished by using metal or polymer tubing or embedding at least one or two or more axially extending elements into the wall of the device shafts, such as elongate wires or ribbons. In some implementations, the extending element may be hollow and protect from abrasion, buckling, or damage at the inputs and outputs of the hubs. In some embodiments, the hollow extending element may be a hollow and flexible coating attached to a hub. The hollow, extending element (e.g., a hollow and flexible coating) may cover a portion of the device shaft when threaded through the hubs. In some embodiments in which the hollow extending element is a coating, the coating may be attached to a portion of a hub such that threading the catheter device through the hub 26, 28, or 30 threads the catheter device through the coating as well. In some implementations, an anti-buckling device may be installed on or about or surrounding a device shaft to avoid misalignment or insertion angle errors between hubs or between a hub and an insertion point. The anti-buckling device may be a laser cut hypotube, a spring, telescoping tubes, tensioned split tubing, or the like.

In some implementations, a number of deflection sensors may be placed along a catheter length to identify buckling. Identifying buckling may be performed by sensing that a hub is advancing distally, while the distal tip of the catheter or interventional device has not moved. In some implementations, the buckling may be detected by sensing that an energy load (e.g., due to friction) has occurred between catheter shafts.

Alternatively, thin tubular stiffening structures can be embedded within or carried over the outside of the device wall, such as a tubular polymeric extrusion or length of hypo-tube. Alternatively, a removable stiffening mandrel may be placed within a lumen in the proximal segment of the device, and proximally removed following distal advance of the hub towards the patient access site, to prevent buckling of the proximal shafts during distal advance of the hub. Alternatively, a proximal segment of one or more of the device shafts may be constructed as a tubular hypo tube, which may be machined (e.g., with a laser) so that its mechanical properties vary along its length. This proximal segment may be formed of stainless steel, nitinol, and/or cobalt chrome alloys, optionally in combination with polymer components which may provide for lubricity and hydraulic sealing. In some embodiments, this proximal segment may be formed of a polymer, such as polyether ether ketone (PEEK), polyether ketone ketone (PEKK), polyethylenimine (PEI), or polyimide (PI). Alternatively, the wall thickness or diameter of the interventional device can be increased in the anti-buckling zone.

In certain embodiments, a device shaft having advanced stiffness (e.g., axially and torsionally) may provide improved transmission of motion from the proximal end of the device shaft to the distal end of the device shaft. For example, the device shafts may be more responsive to motion applied at the proximal end. Such embodiments may be advantageous for robotic driving in the absence of haptic feedback to a user.

In some embodiments, a flexible coating can be applied to a device shaft and/or hub to reduce frictional forces between the device shaft and/or hub and a second device shaft when the second device shaft passes therethrough.

The interventional device hubs may be separated from the support table 20 by sterile barrier 32. Sterile barrier 32 may include a thin plastic membrane such as polyethylene terephthalate (PET), polyethylene terephthalate glycol (PETG), polyethylene terephthalate (PETE), high-density polyethylene (HDPE), polyvinyl chloride (PVC), low-density polyethylene (LDPE), polypropylene (PP), polystyrene (PS), or styrene. This allows the support table 20 and associated drive system to reside on a non-sterile (lower) side of sterile barrier 32. The guidewire hub 26, access catheter hub 28, guide catheter hub 30 and the associated interventional devices are all on a sterile (top) side of the sterile barrier 32. The sterile barrier is preferably waterproof and can also serve as a tray used in the packaging of the interventional devices, discussed further below. The interventional devices can be provided individually or as a coaxially preassembled kit that is shipped and stored in the tray and enclosed within a sterile packaging.

FIGS. 3B-3F schematically illustrate an alternate sterile barrier in the form of a dual function sterile barrier for placement on the support table during the interventional procedure, and shipping tray, having one or more storage channels for carrying sterile interventional devices. The sterile barrier may also act as a sterile work surface for preparation of catheters or other devices during a procedure.

Referring to FIGS. 3B and 3C, there is illustrated a sterile barrier 32 in the form of a pre-shaped tray, for fitting over an elongate support table 20. In use, the elongate support table 20 would be positioned below the sterile barrier 32. The sterile barrier 32 extends between a proximal end 100 and a distal end 102 and includes an upper support surface 104 for supporting the interventional device hubs. In one implementation, the support surface 104 has an axial length greater than the length of the intended interventional devices, in a linear drive configuration.

The length of support surface 104 will typically be at least about 100 centimeters and within the range of from about 100 centimeters to about 2.7 meters. Shorter lengths may be utilized in a system configured to advance the drive couplers along an arcuate path. In some embodiments, two or more support surfaces may be used instead of a single support surface 104. The two or more support surfaces may have a combined length between 100 centimeters to about 2.7 meters. The width of the linear drive table is preferably no more than about 30 to about 80 centimeters.

At least a first channel 106 may be provided, extending axially at least a portion of the length of the support table 20. In the illustrated implementation, first channel 106 extends the entire length of the support table 20. Preferably, the first channel 106 has a sufficient length to hold the interventional devices, and sufficient width and depth to hold the corresponding hubs (for example, by providing lateral support to prevent dislodgment of the hubs when forces are applied to the hubs). First channel 106 is defined within a floor 108, outer side wall 110 and inner side wall 111, forming an upwardly facing concavity. Optionally, a second channel 112 may be provided. Second channel 112 may be located on the same side or the opposite side of the upper support surface 104 from the first channel 106. Two or three or more additional recesses such as additional channels or wells may be provided, to hold additional medical devices or supplies that may be useful during the interventional procedure as well as to collect fluids and function as wash basins for catheters and related devices.

Referring to FIG. 3D, the guide catheter hub 30 is shown positioned on the upper support surface 104, and magnetically coupled to the corresponding coupler holding the drive magnets, positioned beneath the sterile barrier 32. The access catheter hub 28 and access catheter 29, and guidewire hub 26 and guidewire 27 are illustrated residing within the first channel 106 such as before introduction through the guide catheter 31 or following removal from the guide catheter 31.

The interventional devices may be positioned within the channel 106 and enclosed in a sterile barrier for shipping. At the clinical site, an upper panel of the sterile barrier may be removed, or a tubular sterile barrier packaging may be opened and axially removed from the support table 20 and sterile barrier 32 assembly, exposing the sterile top side of the sterile barrier tray and any included interventional devices. The interventional devices may be separately carried in the channel, or preassembled into an access assembly or procedure assembly, discussed in additional detail below.

FIGS. 3D-3F illustrate the support table with sterile barrier in place, and in FIG. 3E, the interventional devices configured in an access assembly for aortic access, following coupling of the access assembly to the corresponding carriages beneath the sterile barrier. The access assembly may be preassembled with the guidewire fully advanced through the access catheter which is in turn fully advanced through the guide catheter. In embodiments in which the access catheter or other catheters are pre-shaped (i.e., pre-curved or not straight), the guidewire and/or outer catheters may be positioned so that relatively stiff sections are not superimposed with curved stiffer sections of the pre-shaped catheter, for example, to avoid creep or straightening of the pre-shaped catheter and/or introduction of a curve into an otherwise straight catheter. This access assembly may be lifted out of the channel 106 and positioned on the support surface 104 for coupling to the respective drive magnets and introduction into the patient. The guide catheter hub 30 is the distal most hub. Access catheter hub 28 is positioned proximally of the guide catheter hub, so that the access catheter 29 can extend distally through the guide catheter. The guidewire hub 26 is positioned most proximally, in order to allow the guidewire 27 to advance through the access catheter 29 and guide catheter 31.

A procedure assembly is illustrated in FIG. 3F following introduction of the procedure assembly through the guide catheter 31 that was used to achieve supra-aortic access. In this implementation, guide catheter 31 remains the distal most of the interventional devices. A first procedure catheter 120 and corresponding hub 122 is illustrated extending through the guide catheter 31. An optional second procedure catheter 124 and corresponding hub 126 is illustrated extending through the first procedure catheter 120. The guidewire 27 extends through at least a portion of the second procedure catheter 124 in a rapid exchange version of second procedure catheter 124, or the entire length of second procedure catheter 124 in an over the wire implementation.

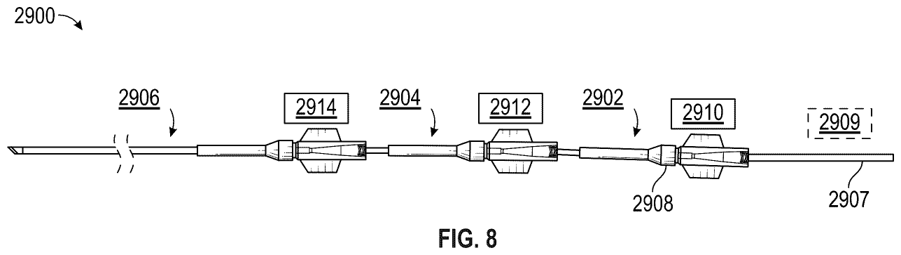

As is discussed in greater detail in connection with FIG. 8, the multi catheter stack may be utilized to achieve both access and the intravascular procedure without the need for catheter exchange. This may be accomplished in either a manual or a robotically driven procedure. In one example, the guide catheter 31 may include a catheter having an inner diameter of at least about 0.08 inches and in one implementation about 0.088 inches. The first procedure catheter 120 may include a catheter having an inner diameter within the range of from about 0.065 inches to about 0.075 inches and in one implementation catheter 120 has an inner diameter of about 0.071 inches. The second procedure catheter 124 may be an access catheter having an OD sized to permit advance through the first procedure catheter 120. The second procedure catheter may be steerable, having a deflection control 2908 configured to laterally deflect a distal end of the catheter. The second procedure (access) catheter may also have an inner lumen sized to allow an appropriately sized guidewire to remain inside the second procedure catheter while performing contrast injections through the second procedure catheter.

In certain embodiments, the catheter 31 may be a “large bore” access catheter or guide catheter having an inner diameter of at least about 0.075 or at least about 0.080 inches in diameter. The catheter 120 may be an aspiration catheter having an inner diameter within the range of from about 0.060 to about 0.075 inches. The catheter 124 may be a steerable catheter with a deflectable distal tip, having an inner diameter within the range of from about 0.025 to about 0.050 inches. The guidewire 27 may have an outer diameter within the range of from about 0.014 to about 0.020 inches. In one example, the catheter 31 may have an inner diameter of about 0.088 inches, the catheter 120 about 0.071 inches, the catheter 124 about 0.035 inches, and the guidewire 27 may have an outer diameter of about 0.018 inches.

In one commercial execution, a preassembled access assembly (guide catheter, access catheter and guidewire) may be carried within a first channel on the sterile barrier tray and a preassembled procedure assembly (one or two procedure catheters and a guidewire) may be carried within the same or a different, second channel on the sterile barrier tray. One or two or more additional catheters or interventional tools may also be provided, depending upon potential needs during the interventional procedure.

FIGS. 3G-3K illustrate embodiments of an alternate sterile barrier having a convex drive surface (e.g., a convex, crowned road like drive surface). FIG. 3G is a cross sectional view of a sterile barrier 232. The sterile barrier 232 includes a convex upper support surface 204. Fluid channels 205 and 207 are positioned laterally of and below the support surface 204 for self-clearing or draining of fluids from the support surface 204 (for example, during an interventional procedure). The fluid channels 205 and 207 may extend axially at least a portion of the length of the sterile barrier.

FIGS. 3I, 3J, and 3K illustrate a sectional perspective view, a cross-sectional view, and a top sectional view, respectively, of a proximal end of the sterile barrier 232. As shown, in FIGS. 3I-3K, the sterile barrier 232 can include a trough 240 in communication with the fluid channels 205 and 207. The trough 240 can receive fluids from the channels 205 and 207 (for example, during an interventional procedure). The trough 240 may be positioned at least partially below the fluid channels 205 and 207 so that fluid within the channels 205 and 207 flows into the trough 240. In certain embodiments, the fluid channels 205 and 207 may be angled relative to a horizontal plane (for example, may decline from an end of the channel furthest from the trough 240 to the trough 240) so that fluid within the channels 205 and 207 is directed to the trough 240. For example, the channels 205 and 207 may increase in depth from an end of the channels furthest from the trough 240 to the trough 240. Alternatively, the sterile barrier 232 and/or support table may be positioned at an angle relative to a horizontal plane, during part of or an entirety of an interventional procedure, such that the end of the channels 205 and 207 furthest from the trough 240 is positioned higher than the trough 240. For example, the sterile barrier 232 and/or support table may be constructed or arranged in an angled arrangement so that an end of the sterile barrier 232 and/or support table opposite the trough 240 is positioned higher than the trough 240. Alternatively or additionally, a drive mechanism may temporarily tilt the sterile barrier 232 and/or support table so that an end of the sterile barrier 232 and/or support table opposite the trough 240 is positioned higher than the trough 240 (for example, by lifting an end of the sterile barrier and/or support table opposite the trough 240 or lowering an end of the sterile barrier 232 and/or support table at which the trough 240 is positioned) so that fluids within the channels 205 and 207 flow into the trough 240.

The trough 240 can include a drain hole 242. The trough 240 can be shaped, dimensioned, and/or otherwise configured so that fluid within the trough 240 empties to the drain hole 242. The drain hole 242 can include tubing, a barb fitting, and/or an on-off valve for removal of fluids from the trough 240. As shown in FIGS. 3I-3K, the trough 240 can be positioned at the proximal end of the sterile barrier 232. In alternate embodiments, the trough 240 may be positioned at a distal end of the sterile barrier 232. In some embodiments, the sterile barrier 232 can include a first trough 240 at the proximal end and a second trough 240 at the distal end. In some embodiments, the trough 240 can also be used as a wash basin.

A first channel 206 may extend axially at least a portion of the length of the sterile barrier 232. The channel 206 can have a sufficient length to hold the interventional devices, and sufficient width and depth to hold the corresponding hubs (for example, by providing support to prevent dislodgement of the hubs when forces are applied to the hubs). Optionally, a second channel 212 may be provided. The second channel 212 may be located on the same side or the opposite side of the upper support surface 204 from the first channel 206. FIG. 3G illustrates the channel 212 located on the opposite side of the support surface 204 from the channel 206. FIG. 3H is a cross-sectional view illustrating an alternate embodiment of the sterile barrier 232 in which the channel 212 is on the same side of the support surface 204 as the channel 206.

As shown in FIGS. 3G and 3H, the channels 206 and 212 can have generally triangular, wedge-shaped, or otherwise angled cross-sections, so as to hold the hubs at an angle relative to a horizontal plane. Holding the hubs at an angle relative to the horizontal plane can allow for a smaller width of the sterile barrier 232.

Two or three or more additional recesses such as additional channels or wells may be provided, to hold additional medical devices or supplies that may be useful during the interventional procedure as well as to collect fluids and function as wash basins for catheters and related devices. In some embodiments, any of the channels or wells described herein may not be part of the sterile barrier, but may instead be part of the drive table positioned below the sterile barrier.

In some embodiments, the sterile barrier 232 can include one or more structural ribs 236. The sterile barrier 232 can further include one or more frame support bosses 228 and 238.

In the embodiment of the sterile barrier 232 shown in FIG. 3G, a width x₁ can be 14 in, about 14 in, between 12 in and 16 in, between 10 in and 18 in, or any other suitable width. In the embodiment of the sterile barrier 232 shown in FIG. 3H, the width x₁ can be 15 in, about 15 in, between 13 in and 17 in, between 11 in and 19 in, or any other suitable width. A height y₁ of the support surface 204 can be 0.125 in, about 0.125 in, between 0.1 and 0.15 in, or any other suitable height. In some embodiments, the support surface 204 can be recessed from a top surface 233 of the sterile barrier 232. A height y₂ between a bottom of the support surface 204 and the top surface 233 can be 0.5 in, about 0.5 in, between 0.25 in and 0.75 in, or any other suitable height. A width x₂ from a lateral edge of the channel 205 to a lateral edge of the channel 207 can be 5 in, about 5 in, between 4 in and 6 in, or any other suitable width. A width X₃ of the support surface 204 can be 4 in, about 4 in, between 3 in and 5 in, or any other suitable width. A height y₃ of the channel 206 and/or channel 212 can be 1.5 in, about 1.5 in, between 1 in and 2 in, or any other suitable height. A width x₄ of the channel 206 and/or channel 212 can be 3 in, about 3 in, between 2 in and 4 in, or any other suitable width. The channel 206 and/or channel 212 can be defined by an arc angle α of 90°, about 90°, between 80° and 100°, or any other suitable angle, and a radius of curvature of 0.125 in, about 0.125 in, between 0.1 and 0.15 in, or any other suitable radius of curvature. In certain embodiments, an arc angle α of 90° or about 90° may be used to hold a hub having a rectangular or generally rectangular cross-section. The support surface 204 can be defined by a radius of curvature of 13 in, about 13 in, between 11 in and 15 in, or any other suitable radius of curvature. The channel 205 and/or channel 207 can be defined by a radius of curvature of 0.25 in, about 0.25 in, between 0.15 in and 0.35 in, or any other suitable radius of curvature.

FIGS. 3L and 3M depict example dimensions of a hub 250 that may be used with the sterile barrier 232 as shown in FIGS. 3G-3K. The hub 250 may be any of the hubs described herein. In certain embodiments, the hub 250 can have a width w₁ of 3.75 in, about 3.75 in, between 3.25 in and 4.25 in, or any other suitable width. The hub 250 can have a height h₁ of 1.5 in, about 1.5 in, between 1.25 in and 1.75 in, or any other suitable height. Alternatively, the hub 250 can have a height h₂ of 2 in, about 2 in, between 1.75 in and 2.25 in, or any other suitable height. In some embodiments, the hub 250 can have a length L₁ of 2.5 in, about 2.5 in, between 2 in and 3 in or any other suitable length. Alternatively, the hub 250 can have a length L₂ of 4 in, about 4 in, between 3.25 in and 4.75 in, or any other suitable length.

In some embodiments, a top surface of the support table can include surface features that generally correspond to those of the sterile barrier 232. For example, the support table can include a convex surface configured to correspond to the shape, size, and location of the support surface 204 and/or one or more recesses configured to correspond to the shape, size, and location of the channels 205 and 207.

In alternate embodiments, a planar support surface (for example, support surface 104 of sterile barrier 32) can be positioned at an angle to a horizontal plane to facilitate the draining of fluids. In some embodiments, the sterile barrier and/or support table may be positioned, during part of or the entirety of an interventional procedure, at an angle to a horizontal plane to facilitate the draining of fluids. For example, the sterile barrier and/or support table may be constructed or arranged in an angled arrangement (for example, so that one lateral side of the planar support surface is positioned higher than the other lateral side of the planar support surface, the proximal end is higher than the distal end, or the distal end is higher than the proximal end) to facilitate the drainage of fluids. Alternatively or additionally, a drive mechanism may temporarily tilt the sterile barrier and/or support table (for example, so that one lateral side of the planar support surface is positioned higher than the other lateral side of the planar support surface, the proximal end is higher than the distal end, or the distal end is higher than the proximal end) to facilitate the drainage of fluids. For example, the drive mechanism may raise or lower one lateral side of the sterile barrier and/or support table, the proximal end of the sterile barrier and/or support table, and/or the distal end of the sterile barrier and/or support table.

In certain embodiments, a support surface (for example, support surface 104 of sterile barrier 32) can be positioned in a vertical configuration instead of the horizontal configuration shown, for example, in FIGS. 3A-3F. For example, the support surface 104 can be positioned at about 90 degrees (or any other suitable angle) from a horizontal plane (e.g., rotated 90 degrees about a long axis of the support surface 104 relative to the embodiment shown in FIGS. 3A-3F). A vertical configuration may provide for easier interaction with the drive system 18 by a physician, A vertical configuration may also provide for a lower axis of catheter travel closer to a patient without adding standoff height to the drive system 18.

In some embodiments, the drive system 18 may be positioned, during part of or the entirety of an interventional procedure, at an angle to a horizontal plane to facilitate the draining of fluids. For example, the drive system 18 may be constructed or arranged in an angled arrangement (for example, so that one lateral side of the planar support surface is positioned higher than the other lateral side of the planar support surface, the proximal end is higher than the distal end, or the distal end is higher than the proximal end) to facilitate the drainage of fluids. Alternatively or additionally, a drive mechanism may temporarily tilt the drive system 18 (for example, so that one lateral side of the drive system 18 is positioned higher than the other lateral side of the drive system 18, the proximal end is higher than the distal end, or the distal end is higher than the proximal end) to facilitate the drainage of fluids. For example, the drive mechanism may raise or lower one lateral side of the system 18, the proximal end of the drive system 18, and/or the distal end of the drive system 18. In some embodiments, the drive system 18 may be angled so that it extends at an angle away from axis point 24 (for example, so that the proximal end is higher than the distal end), for example, to allow for clearance of a patient's feet.

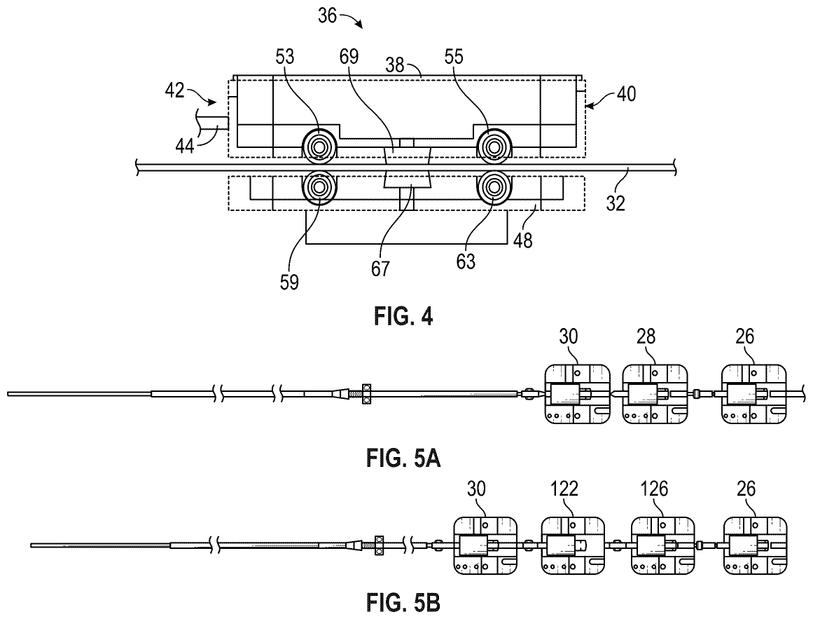

Referring to FIG. 4, hub 36 may represent any of the hubs previously described. Hub 36 includes a housing 38 which extends between a proximal end 40 and a distal end 42. An interventional device 44, which could be any of the interventional devices disclosed herein, extends distally from the hub 36 and into the patient 14 (not illustrated). A hub adapter 48 or carriage acts as a shuttle by advancing proximally or distally along a track in response to operator instructions or controller manipulations. The hub adapter 48 includes at least one drive magnet 67 configured to couple with a driven magnet 69 carried by the hub 36. This provides a magnetic coupling between the drive magnet 67 and driven magnet 69 through the sterile barrier such that the hub 36 is moved across the top of the sterile barrier 32 in response to movement of the hub adapter 48 outside of the sterile field. Movement of the hub adapter is driven by a drive system carried by the support table and described in additional detail below. The hub adapter may act as a robotic drive for an interventional device coupled thereto.

To reduce friction in the system, the hub 36 may be provided with at least a first roller 53 and a second roller 55 which may be in the form of wheels or rotatable balls or drums. The rollers space the sterile barrier apart from the surface of the driven magnet 69 by at least about 0.02 centimeters (about 0.008 inches) and generally no more than about 0.08 centimeters (about 0.03 inches). In some implementations, the space is within the range of from about 0.03 centimeters (about 0.010 inches) to about 0.041 centimeters (about 0.016 inches). The space between the drive magnet 67 and driven magnet 69 is generally no more than about 0.38 centimeters (about 0.15 inches) and in some implementations is no more than about 0.254 centimeters (about 0.10 inches) such as within the range of from about 0.216 centimeters (about 0.085 inches) to about 0.229 centimeters (about 0.090 inches). The hub adapter 48 may similarly be provided with at least a first hub adapter roller 59 and the second hub adapter roller 63, which may be positioned opposite the respective first roller 53 and second roller 55 as illustrated in FIG. 4.

In other embodiments, any of the interventional devices and/or hubs described herein may be coupled to a carriage or hub adapter via a mechanical coupling.

Additional details regarding a magnetic coupling through a sterile barrier can be found in U.S. patent application Ser. No. 18/678,766, entitled MAGNETIC COUPLING THROUGH A STERILE FIELD BARRIER, filed May 30, 2024, and U.S. patent application Ser. No. 19/406,652, entitled MAGNETIC COUPLING THROUGH A STERILE FIELD BARRIER, filed Dec. 2, 2025, the entirety of each of which is hereby incorporated by reference herein.

Any of the catheters illustrated, for example, in FIG. 5A, 5B or 11 generally include an elongate tubular body extending between a proximal end and a distal functional end. The length and diameter of the tubular body depends upon the desired application. For example, lengths in the area of from about 90 centimeters to about 195 centimeters or more are typical for use in femoral access percutaneous transluminal coronary applications. Intracranial or other applications may call for a different catheter shaft length depending upon the vascular access site.

Certain embodiments of hub assemblies described herein, such as hub assembly (“hub”) 36 shown in FIG. 4, include a housing (e.g., housing 38) for coupling an interventional device thereto, components (e.g., rollers 53 and 55) for directly coupling to and moving along a drive table, and magnet(s) (e.g., magnet 69) for magnetically coupling to a hub adapter across a sterile barrier. A hub (or hub assembly) can refer to a single assembly with a housing, or a hub (or hub assembly) can generally refer to an apparatus having two (or more) subassemblies (e.g., a first subassembly and a second subassembly). In some embodiments of a hub assembly having two subassemblies, a hub can refer to a first subassembly that can be configured to couple to and house an interventional device, and that may be removably attachable to a second subassembly (or mount) configured to magnetically couple to a hub adapter across a sterile barrier and move along a drive table. Such a hub and mount may together form a hub assembly. Such hub assemblies may allow for a hub (first subassembly) to be removed from a mount (second subassembly) which can be advantageous, for example, so that a different hub can be coupled to the same mount or so that the hub may be used separately from the mount (e.g., for a manual procedure).

An arrangement of a hub assembly having a hub that is releasably couplable to a mount can allow for replacement of a hub with a different hub having a different interventional device coupled thereto without breaking a magnetic connection with a hub adapter. For example, such an arrangement may allow for a hub coupled to an access catheter to be removed from a mount and replaced with a hub coupled to a procedure catheter without breaking a magnetic connection between active and passive magnetic sides of the coupling of the hub adapter and hub assembly (e.g., between the hub adapter and the mount). In some embodiments, the mount may be a magnetically driven member, an axially driven member, a puck, a slider, a shuttle, or a stage. The robotic control systems described herein can relate to various embodiments of systems that include a hub, or a hub and a mount, regardless of whether they are described in reference to a hub, or a hub and mount, unless explicitly indicated or indicated by context. In some embodiments, a mount may be magnetically coupled to a hub adapter across a sterile barrier prior to coupling a hub to the mount, for example, when preparing the drive table for a medical procedure.

As described herein, the hub assemblies can include intravascular devices that can access the vascular system of a patient via at least one artery and/or vein (e.g., the femoral artery) and be driven within the vascular system to perform a vascular procedure.

Additional details regarding hub assemblies can be found in U.S. patent application Ser. No. 18/986,519, entitled ROBOTIC HUB ASSEMBLY, filed Dec. 18, 2024, which is hereby expressly incorporated by reference in its entirety herein.

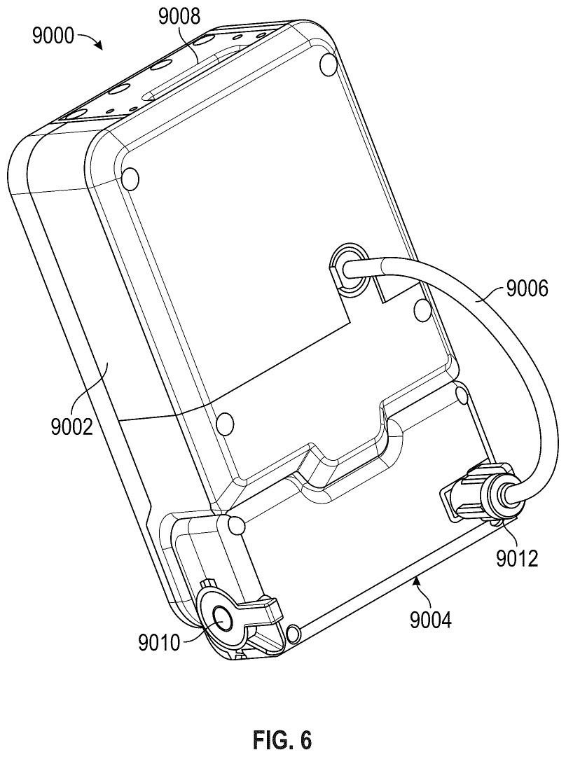

FIG. 6 illustrates a hub assembly 9000. The hub assembly 9000 may include any of the same or similar features and/or functions as the other embodiments of hubs and hub assemblies described herein and vice versa.

In some embodiments, the hub assembly 9000 can include a first subassembly, puck, or mount 9002 and a second subassembly or hub 9004. The mount 9002 can also be referred to as a catheter puck, a hub mount, and/or a first hub member. The mount 9002 can be configured to couple to and move along a drive table. The hub assembly 9000 can be configured to be positioned on a sterile side (e.g., a disposable equipment side) of a sterile barrier.

In some embodiments, the hub 9004 can be referred to as a second hub member. The hub 9004 may include or couple to an interventional device, such as a catheter or guidewire.

As described herein, in certain embodiments, an interventional device may be coupled to a fluidics management system (e.g., to receive fluids such as contrast or saline, or for aspiration). In some embodiments, the mount 9002 can be coupled to the fluidics management system. In some embodiments, a fluidics connector 9006 can extend between and fluidly couple the mount 9002 and the hub 9004.

The mount 9002 can further include a first housing. The first housing can define one or more openings 9008 and a plurality of internal components. The first housing can form an outer shell to protect the internal components of the mount 9002. The first housing can include at least one side shaped and/or dimensioned (e.g., having a contour) for receiving the hub 9004.

The one or more openings 9008 can provide access for fluidics and/or electrical connections into the mount 9002. In some embodiments, a contrast tube, a saline tube, and/or an aspiration tube may extend through the one or more openings 9008 into the mount 9002. Additionally, in some embodiments, a power line may extend through the one or more openings 9008 to provide electrical power into the mount 9002. The mount 9002 can be configured to receive an input from one or more active torque elements of an active torque subsystem. In some embodiments, the inputs from the one or more active torque elements may be a magnetic rotary force as described herein. The mount 9002 can be configured to transmit one or more outputs to the hub 9004. In some embodiments, the mount 9002 may transform one or more rotary inputs of the one or more active torque elements into corresponding linear and/or rotary outputs. In some embodiments, the mount 9002 may be configured to translate linearly along a drive table (e.g., in response to linear movement of a hub adapter within the drive table due to a magnetic coupling between mount 9002 and the hub adapter).

The hub 9004 can further include a second housing. The hub 9004 can include a lumen 9010 for receiving an interventional device therein. The hub 9004 can include a luer 9012. The hub 9004 can further include a plurality of internal components described in greater detail below. The second housing can form an outer shell to protect the internal components of the hub 9004. In some embodiments, the second housing may include at least one side shaped and/or dimensioned (e.g., having a contour) to correspond to the shape of the first housing. For example, the contour of the second housing can correspond to the contour of the first housing of the mount 9002. The hub 9004 can be configured to receive one or more inputs from the mount 9002. The hub 9004 can be configured to transmit one or more outputs. In some embodiments, the hub 9004 may transform the outputs of the mount 9002 into corresponding linear and/or rotary motion of components within or coupled to the hub 9004 (e.g., the interventional device coupled to the hub 9004 and/or one or more fluidics components).

The fluidics connector 9006 can be a tubular body defining an interior lumen extending from one end of the fluidics connector 9006 to a second end of the fluidics connector 9006. In some embodiments, the fluidics connector 9006 may be configured to transport fluids between the mount 9002 and the hub 9004. For example, the fluidics connector 9006 may facilitate the flow of contrast, saline, bodily fluids, and/or air between the mount 9002 and the hub 9004. The fluidics connector 9006 can transport fluids from the mount 9002 to the hub 9004, or vice versa. The fluidics connector 9006 may form an airtight seal.

The hub 9004 may be removably coupled to the mount 9002. In some embodiments, the hub 9004 can be mounted to a mounting element defined by the mount 9002. The fluidics connector 9006 may be coupled to both the mount 9002 and the hub 9004. In some embodiments, the hub 9004 may be in fluid communication with the mount 9002 via the fluidics connector 9006. Accordingly, fluids may be transferred between the mount 9002 and the hub 9004 via the fluidics connector 9006.

Additional details for the fluidics system are described in U.S. patent application Ser. No. 18/666,217, entitled FLUIDICS CONTROL SYSTEM FOR MULTI CATHETER STACK, filed May 16, 2024, the entirety of which is hereby incorporated by reference herein.

Any of a variety of sensors may be provided on any of the catheters, hubs, carriages, or table, depending upon the desired data. For example, in some implementations, it may be desirable to measure axial tension or compression force applied to the catheter such as along a force sensing zone. It may also be desirable to measure elastic forces across the magnetic coupling between the hub and corresponding carriage, using the natural springiness (compliance) of the magnetic coupling to measure the force applied to the hub. The magnetic coupling between the hubs and carriages creates a spring. When a force is applied to the hub, the hub will move a small amount relative to the carriage. In robotics, this is called a series elastic actuator. This property can be used to measure the force applied from the carriage to the hub. To measure the force, the relative distance between the hub and the carriage is determined and characterize some effective spring constant k between the two components.

The relative distance could be measured in multiple different ways. One method for measuring the relative distance between the hub and carriage is a magnetic sensor (e.g., a Hall effect Sensor between hub and carriage). A magnet is mounted to either the hub or carriage, and a corresponding magnetic sensor is mounted on the other device (carriage or hub). The magnetic sensor might be a hall effect sensor, a magnetoresistive sensor, or another type of magnetic field sensor. Generally, multiple sensors may be used to increase the reliability of the measurement. This reduces noise and reduces interference from external magnetic fields.

Other non-contact distance sensors can also be used. These include optical sensors, inductance sensors, and capacitance sensors. Optical sensors would preferably be configured in a manner that avoids accumulation of blood or other fluid in the interface between the hubs and carriages. In some implementations, wireless (i.e., inductive) power may be used to translate movement and/or transfer information across the sterile barrier between a drive carriage and a hub, for example.

The magnetic coupling between the hub and the carriage has a shear or axial break away threshold which may be about 300 grams or 1000 grams or more. The processor can be configured to compare the axial force applied to the catheter to a preset axial trigger force which, if applied to the catheter, is perceived to create a risk to the patient. If the trigger force is reached, the processor may be configured to generate a response such as a visual, auditory or tactile feedback to the physician, and/or intervene and shut down further advance of the catheter until a reset is accomplished. An override feature may be provided so the physician can elect to continue to advance the catheter at forces higher than the trigger force, in a situation where the physician believes the incremental force is warranted.

Force and/or torque sensing fiber optics (e.g., Fiber Bragg Grating (FBG) sensors) may be built into the catheter side wall to measure the force and/or torque at various locations along the shaft of a catheter or alternatively may be integrated into a guidewire. The fiber measures axial strain, which can be converted into axial force or torque (when wound helically). At least a first FBG sensor can be integrated into a distal sensing zone, proximal sensing zone and/or intermediate sensing zone on the catheter or guidewire, to measure force and/or torque in the vicinity of the sensor.

It may also be desirable to understand the three-dimensional configuration of the catheter or guidewire during and/or following transvascular placement. Shape sensing fiber optics, such as an array of FBG fibers, can sense the shape of catheters and guidewires. By using multiple force sensing fibers that are a known distance from each other, the shape along the length of the catheter/guidewire can be determined.

A resistive strain gauge may be integrated into the body of the catheter or guidewire to measure force or torque, such as at the distal tip and/or proximal end of the device.

Measurements of force and/or torque applied to the catheter or guidewire shafts can be used to determine applied force and/or torque above a safety threshold. When an applied force and/or torque exceeds a safety threshold, a warning may be provided to a user. Applied force and/or torque measurements may also be used to provide feedback related to better catheter manipulation and control. Applied force and/or torque measurements may also be used with processed fluoroscopic imaging information to determine or characterize distal tip motion.

Absolute position of the hubs (and corresponding catheters) along the length of the table may be determined in a variety of ways. For example, a non-contact magnetic sensor may be configured to directly measure the position of the hubs through the sterile barrier. The same type of sensor can also be configured to measure the position of the carriages. Each hub may have at least one magnet attached to it. The robotic table would have a linear array of corresponding magnetic sensors going the entire length of the table. A processor can be configured to determine the location of the magnet along the length of the linear sensor array, and display axial position information to the physician.

The foregoing may alternatively be accomplished using a non-contact inductive sensor to directly measure the position of the hubs through the sterile barrier. Each hub or carriage may be provided with an inductive “target” in it. The robotic table may be provided with an inductive sensing array over the entire working length of the table. As a further alternative, an absolute linear encoder may be used to directly measure the linear position of the hubs or carriages. The encoder could use any of a variety of different technologies, including optical, magnetic, inductive, and capacitive methods.

In one implementation, a passive (no electrical connections) target coil may be carried by each hub. A linear printed circuit board (PCB) may run the entire working length of the table (e.g., at least about 1.5 meters to about 1.9 meters) configured to ping an interrogator signal which stimulates a return signal from the passive coil. The PCB is configured to identify the return signal and its location.

Axial position of the carriages may be determined using a multi-turn rotary encoder to measure the rotational position of the pulley, which directly correlates to the linear position of the carriage. Direct measurement of the location of the carriage may alternatively be accomplished by recording the number of steps commanded to the stepper motor to measure the rotational position of the pulley, which directly correlates to the linear position of the carriage.

The location of the catheters and guidewires within the anatomy may also be determined by processing the fluoroscopic image with machine vision, such as to determine the distal tip position, distal tip orientation, and/or guidewire shape. Comparing distal tip position or movement or lack thereof to commanded or actual proximal catheter or guidewire movement at the hub, may be used to detect a loss of relative motion, which may be indicative of a device shaft buckling, prolapse, kinking, or a similar outcome (for example, along the device shaft length inside the body (e.g., in the aorta) or outside the body between hubs). The processing may be done in real time to provide position/orientation data at up to 30 Hertz, although this technique would only provide data while the fluoroscopic imaging is turned on. In some embodiments, machine vision algorithms can be used to generate and suggest optimal catheter manipulations to access or reach anatomical landmarks, similar to driver assist. The machine vision algorithms may utilize data to automatically drive the catheters depending on the anatomy presented by fluoroscopy.

Proximal torque applied to the catheter or guidewire shaft may be determined using a dual encoder torque sensor.

Confirming the absence of bubbles in fluid lines may also be accomplished using bubble sensors, particularly where the physician is remote from the patient. This may be accomplished using a non-contact ultrasonic sensor that measures the intensity and Doppler shift of the reflected ultrasound through the sidewall of fluid tubing to detect bubbles and measure fluid flow rate or fluid level. An ultrasonic or optical sensor may be positioned adjacent an incoming fluid flow path within the hub, or in a supply line leading to the hub. To detect the presence of air bubbles in the infusion line (that is formed of ultrasonically or optically transmissive material) the sensor may include a signal source on a first side of the flow path and a receiver on a second side of the flow path to measure transmission through the liquid passing through the tube to detect bubbles. Alternatively, a reflected ultrasound signal may be detected from the same side of the flow path as the source due to the relatively high echogenicity of bubbles.

In some embodiments, a bubble removal system is automatically activated upon detection of in line bubbles. A processor may be configured to activate a valve positioned in the flow path downstream of the bubble detector, upon the detection of bubbles. The valve diverts a column of fluid out of the flow path to the patient and into a reservoir. Once bubbles are no longer detected in the flow path and after the volume of fluid in the flow path between the detector and the valve has passed through the valve, the valve may be activated to reconnect the source of fluid with the patient through the flow path. In other embodiments, the bubble removal system can include a pump and control system upstream of the bubble detector for removal of in line bubbles. A processor may be configured to activate the pump upon detection of bubbles to reverse the fluid flow and clear the bubbles into a waste reservoir before reestablishing bubble free forward flow.

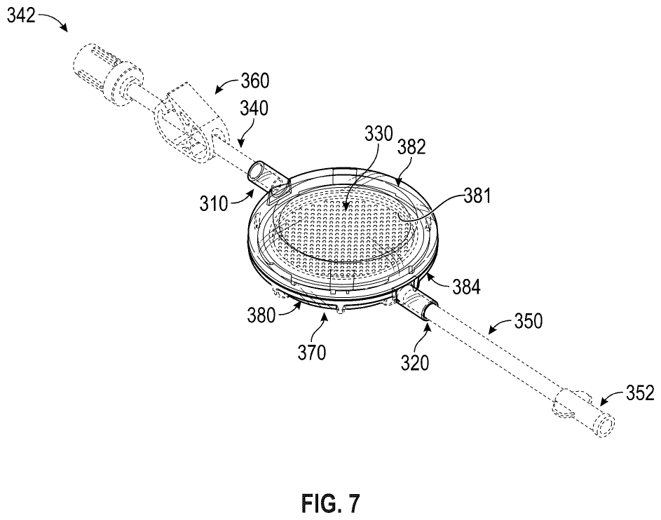

It may additionally be desirable for the physician to be able to view aspirated clot at a location within the sterile field and preferably as close to the patient as practical for fluid management purposes. This may be accomplished by providing a clot retrieval device mounted on the hub, or in an aspiration line leading away from the hub in the direction of the pump. Referring to FIG. 7, one example of a clot retrieval device 370 can include a body 380 enclosing a chamber 381 which communicates with a first port 310 and a second port 320.

In some embodiments, the body 380 includes a housing having a top portion 382 and a bottom portion 384. The body 380 may include a filter 330 positioned in the chamber 381 between the top portion 382, and the bottom portion 384. In some examples, the first port 310 is configured to connect to a first end of a first tube 340 that is fluidly connected to a proximal end of an aspiration catheter.

In an embodiment that is configured to be connected downstream from the hub, the first tube 340 includes a connector 342 positioned at a second end of the first tube 340 that is configured to engage or mate with a corresponding connector on or in communication with the hub. The first port 310 directly communicates with the chamber on the upstream (e.g., top side) of the filter, and the second port 320 directly communicates with the chamber on the downstream (e.g., bottom side) of the filter to facilitate direct visualization of material caught on the upstream side of the filter.

In an implementation configured for remote operation, any of a variety of sensors may be provided to detect clot passing through the aspiration line and/or trapped in the filter, such as an optical sensor, pressure sensor, flow rate sensor, ultrasound sensor or others known in the art.

In some embodiments, the second port 320 is configured to connect to a first end of a second tube 350 that is fluidly connected to an aspiration source (e.g., a pump). In some embodiments, the second tube 350 includes a connector 352 positioned at a second end of the second tube 350 that is configured to engage or mate with a corresponding connector on the pump.

In some examples, the system 300 can include an on-off valve 360, such as a clamp. The clamp can be positioned in between the filter 330 and the patient, such as over the first tube 340, to allow the user to engage the clamp and provide flow control by isolating the patient from the clot retrieval device 370. Closing the valve 360 and operating the remote vacuum pump (not illustrated) causes the canister associated with the vacuum pump and the chamber 381 to reach the same low pressure. Due to the short distance and small line volume of the lumen between the chamber 381 and the distal end of the catheter, a sharp negative pressure spike is experienced at the distal end of the catheter rapidly following opening of the valve 360. Additional details are disclosed in U.S. Pat. No. 11,259,821 , issued Mar. 1, 2022 to Buck et al., entitled Aspiration System with Accelerated Response, the entirety of which is hereby expressly incorporated by reference herein. In some embodiments, a vacuum may be cycled against a clot to retrieve the clot. The vacuum may be automatically and robotically controlled to remove the clot.

The body 380 can have a top surface spaced apart from a bottom surface by a tubular side wall. In the illustrated implementation, the top and bottom surfaces are substantially circular and spaced apart by a cylindrical side wall. The top surface may have a diameter that is at least about three times, or five times or more than the axial length (transverse to the top and bottom surfaces) of the side wall, to produce a generally disc shaped housing. Preferably, at least a portion of the top wall is optically transparent to improve clot visualization once it is trapped in the clot retrieval device 370. Additional details may be found in U.S. patent application Ser. No. 18/436,882, entitled Device For Clot Retrieval With Varying Tube Diameters, filed Feb. 8, 2024, the entirety of which is hereby expressly incorporated by reference herein.

In some examples, the body 380 can include a flush port (not illustrated) that is configured to allow the injection of an optically transparent media, such as air, saline, or other fluid, into the chamber 381 to clear an optical path between the window and the filter to improve clot visualization once it is trapped in the filter 330.

The foregoing represents certain specific implementations of a drive table and associated components and catheters. A wide variety of different drive table constructions can be made, for supporting and axially advancing and retracting two or three or four or more drive magnet assemblies to robotically drive interventional devices, fluid elements, and electrical umbilical elements for communicating electrical signals and fluids to the catheter hubs, as will be appreciated by those of skill in the art in view of the disclosure herein. Additional details may be found in U.S. patent application Ser. No. 17/527,393, entitled CATHETER DRIVE SYSTEM FOR SUPRA-AORTIC ACCESS, filed Nov. 16, 2021, the entirety of which is hereby incorporated by reference herein. Additional details may be found in U.S. patent application Ser. No. 18/060,935, entitled “METHOD OF PRIMING AN INTERVENTIONAL DEVICE ASSEMBLY,” filed Dec. 1, 2022, the entirety of which is hereby expressly incorporated by reference herein.

While the foregoing describes robotically driven interventional devices and manually driven interventional devices, the devices may be manually driven, robotically driven, or a combination of both manually and robotically driven interventional devices, as will be appreciated by those of skill in the art in view of the disclosure herein.

FIG. 8 illustrates a side elevational schematic view of a multi-device assembly 2900 (e.g., multi-catheter interventional device assembly) for combined supra-aortic access and/or neurovascular site access and procedure (e.g., aspiration), as described herein. The multi-device assembly 2900 may be configured for either a manual or a robotic procedure.

The multi-device assembly 2900 includes an insert or access catheter 2902, a procedure catheter 2904, and a guide catheter 2906. Other components are possible, including, but not limited to, one or more guidewires (e.g., optional guidewire 2907), one or more guide catheters, an access sheath, and/or one or more other procedure catheters and/or associated catheter (control) hubs. In some embodiments, the assembly 2900 may also be configured with an optional deflection control 2908 for controlling deflection of one or more catheters of assembly 2900.

In operation, the multi-device assembly 2900 may be used without having to exchange hub components. For example, in the two stage procedure disclosed previously, a first stage for achieving supra-aortic access includes mounting an access catheter, guide catheter and guidewire to the support table. Upon gaining supra aortic access, the access catheter and guidewire were typically removed from the guide catheter. Then, a second catheter assembly is introduced through the guide catheter after attaching a new guidewire hub and a procedure catheter hub to the corresponding drive carriage on the support table.

The devices of the multi-device assembly 2900 may be operated using a control mechanism (e.g., a handheld controller, a user interface, etc.) as described in further detail herein. The control mechanism may be operated to cause independent or simultaneous axial translation of the interventional devices of the multi-device assembly 2900 (e.g., along a drive surface). The control mechanism may be operated to cause independent or simultaneous rotational movement of one or more of the interventional devices of the multi-device assembly 2900.

The single multi-device assembly 2900 of FIG. 8 is configured to be operated without having to remove hubs and catheters and without the addition of additional assemblies and/or hubs. Thus, the multicomponent access and procedure configuration of assembly 2900 may utilize a guidewire 2907 manufactured to function as an access guidewire and a navigation guidewire to allow for sufficient access and support, and navigation to the particular distal treatment site. In a non-limiting example configured for robotic implementation, a catheter assembly may include a guidewire hub (e.g., guidewire hub 2909 or guidewire hub 26 positioned on a drive table and to the right of catheter 2902), an insert or access catheter hub 2910, a procedure catheter hub 2912, a guide catheter hub 2914 and corresponding catheters. In certain embodiments, one or more of the hubs may include or be coupled to a hemostasis valve (e.g., a rotating hemostasis valve) to accommodate introduction of interventional devices therethrough. In some embodiments, any of the control mechanisms described herein can include at least one control for opening and closing a hemostasis valve.

Additional details regarding hemostasis valves and fluidics systems are included in U.S. patent application Ser. No. 17/879,614, entitled Multi Catheter System With Integrated Fluidics Management, filed Aug. 2, 2022, U.S. patent application Ser. No. 18/666,217, entitled Fluidics Control System For Multi Catheter Stack, filed May 16, 2024, and U.S. patent application Ser. No. 19/394,754, entitled Fluidics Control System For Multi Catheter Stack, filed Nov. 19, 2025, each of which is hereby expressly incorporated by reference in its entirety herein.

One or more of interventional device and hub combinations may further include fluidics connections for coupling to fluid sources and/or vacuum sources. For example, each of the insert or access catheter 2902, the procedure catheter 2904, and the guide catheter 2906 may be in fluid communication with a saline source, a contrast source, and/or a vacuum source. In some embodiments, any of the control mechanisms described herein can include at least one control for initiating and/or terminating the introduction of fluids to one or more of the catheters and/or aspiration of fluids from one or more of the catheters. For example, any of the control mechanisms described herein can include at least one control for opening and/or closing one or more valves to initiate the introduction of fluids to one or more of the catheters and/or aspiration of fluids from one or more of the catheters. For example, any of the control mechanisms described herein can be used to control various components (e.g., manifold valves, pumps, hemostatic valves, hubs, and/or catheters) of a fluidics system as described in U.S. patent application Ser. No. 17/879,614, entitled Multi Catheter System With Integrated Fluidics Management, filed Aug. 2, 2022, the entirety of which is hereby incorporated by reference herein.

In some embodiments, the control mechanisms described herein may allow a user to simultaneously control movement of a catheter or other interventional device (e.g., axial and/or rotational movement) and a fluidics system (e.g., for introduction of fluids and/or aspiration).

In some implementations, other control operations beyond translational movement and rotational movement may be carried out using any of the controls described herein. For example, the controls may be configured to drive a shape change and/or stiffness change of a corresponding interventional device. Controls may be toggled between different operating modes. For example, controls may be toggled between movement driven by acceleration and velocity to movement that reflects actual linear displacement or rotation.

In some implementations, the control mechanisms may be provided with a visual display or other indicator of the relative positions of the controls which may correspond to the relative positions of the interventional devices. Such displays may depict any or all movement directions, instructions, percentage of movements performed, and/or hub and/or catheter indicators to indicate which device is controlled by a particular control. In some implementations, the display may depict applied force or resistance encountered by the catheter or other measurement being detected or observed by a particular hub or interventional component.

The systems described herein may compare an actual fluoroscopic image position to an input displacement from the controller. A static fluoroscopic image of the patient may be captured in which the patient's vasculature is indexed relative to bony landmarks or one or more implanted soft tissue fiducial markers. Then a real time fluoroscopic image may be displayed as an overlay, aligned with the static image by registration of the fiducial markers. Visual observation of conformance of the real time movement with the static image, assisted by detected force data can help confirm proper navigation of the associated catheter or guidewire. The systems described herein can also display a comparison of an input proximal mechanical translation of a catheter or guidewire and a resulting distal tip output motion or lack thereof. A loss of relative motion at the distal tip may indicate shaft buckling, prolapse, kinking, or a similar outcome, either inside or outside the body. Such a comparison may be beneficial when the shaft buckling, prolapse, kinking, or similar outcome occurs outside of a current fluoroscopic view.

Additional details regarding a controller for a robotic surgical system can be found in U.S. patent application Ser. No. 18/784,630, entitled SYSTEM FOR REMOTE MEDICAL PROCEDURE, filed Jul. 25, 2024, the entirety of which is hereby incorporated by reference herein.

Once access above the aortic arch has been achieved, the insert or access catheter 2902 (associated with insert or access catheter hub 2910) may be parked in the vicinity of a carotid artery ostia and the remainder or a subset of the catheter assembly may be guided more distally toward a particular site (e.g., a clot site, a surgical site, a procedure site, etc.).

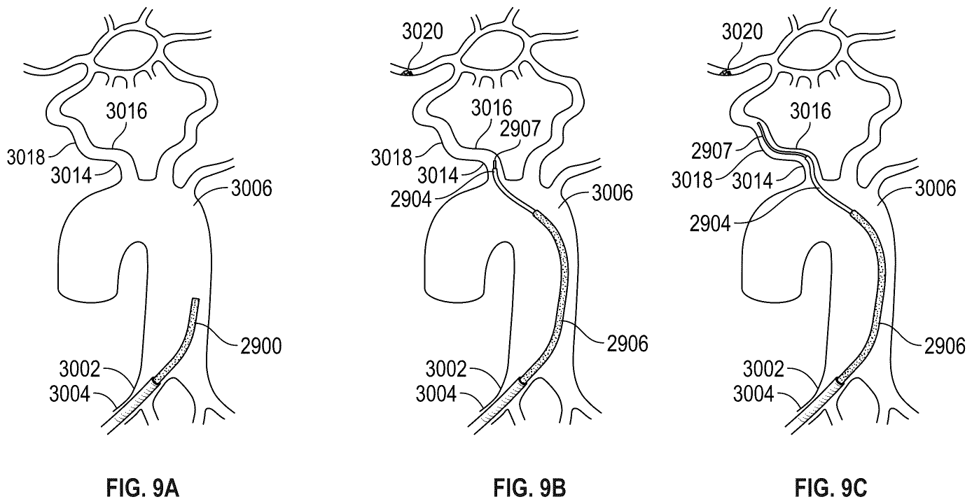

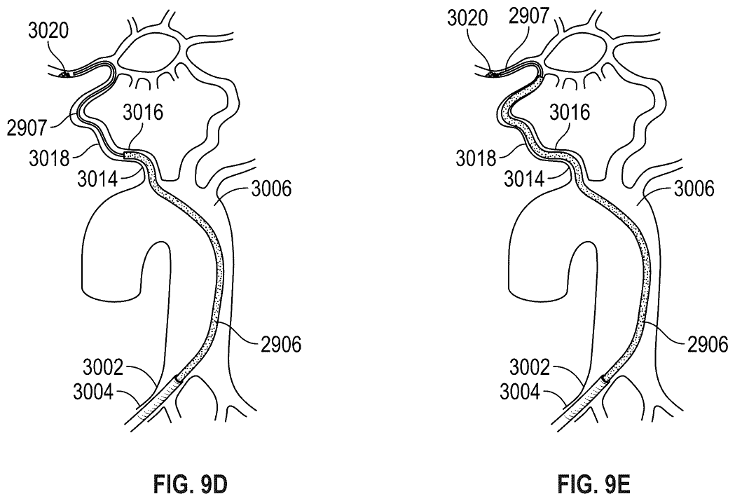

In some embodiments, other smaller procedure catheters may also be added and used at the site. As used herein for multi-device assembly 2900, in a robotic configuration of assembly 2900, the catheter 2906 may function as a guide catheter. The catheter 2904 may function as a procedure (e.g., aspiration) catheter. In some embodiments, the catheter 2906 may function to perform aspiration in addition to functioning as a guide catheter, either instead of or in addition to the catheter 2904. The access catheter 2902 may have a distal deflection zone and can function to access a desired ostium. One of skill in the art will appreciate from FIGS. 9A-9E that either manual manipulation or robotic manipulation of the multi catheter stack are contemplated herein.

In some embodiments, the multi-device assembly 2900 (or other combined catheter assemblies described herein) may be driven as a unit to a location. However, each catheter (or guidewire) component may instead be operated and driven independent of one another to the same or different locations.

In a non-limiting example, the multi-device assembly 2900 may be used for a diagnostic angiogram procedure. In some embodiments, the assembly 2900 may include only the guidewire 2907 and access catheter 2902 (in the form of a diagnostic angiographic catheter) for performing the diagnostic angiogram procedure or only the guidewire 2907 and the access catheter 2902 may be utilized during the procedure. Alternatively, the guide catheter 2906 and procedure catheter 2904 may be retracted proximally to expose the distal end of the access catheter 2902 (e.g., a few centimeters of the distal end of the access catheter) to perform the diagnostic angiography.

As shown in FIG. 8, the guide catheter 2906, procedure catheter 2904, access catheter 2902, and guidewire 2907 can be arranged concentrically. In certain embodiments, the guide catheter 2906 may be a ‘large bore’ guide catheter or access catheter having an inner diameter of at least about 0.075 or at least about 0.080 inches in diameter. The procedure catheter 2904 may be an aspiration catheter having an inner diameter within the range of from about 0.060 to about 0.075 inches. The access catheter 2902 may be a steerable catheter with a deflectable distal tip, having an inner diameter within the range of from about 0.025 to about 0.050 inches. The guidewire 2907 may have an outer diameter within the range of from about 0.014 to about 0.020 inches. In one example, the guide catheter 2906 may have an inner diameter of about 0.088 inches, the procedure catheter 2904 about 0.071 inches, the access catheter 2902 about 0.035 inches, and the guidewire 2907 may have a diameter of about 0.018 inches.

FIGS. 9A-9E depict an example sequence of steps of introducing a multi-catheter assembly configured to achieve access all the way to the clot, either manually or robotically. FIGS. 9A-9E may be described using the interventional device assembly of FIG. 8. Other combinations of catheters may be substituted for the interventional device assembly, as will be appreciated by those of skill in the art in view of the disclosure herein.

Referring to FIG. 9A, the three catheter multi-device assembly 2900 is shown driven through an introducer sheath 3002, up through the iliac artery 3004 and into the descending aorta. Next, the access catheter 2902, the procedure catheter 2904 (e.g., 0.071 inch) and the guide catheter 2906 (e.g., 0.088 inch) are tracked up to the aortic arch 3006, as shown in FIG. 9B. Here, the distal end of the guide catheter 2906 may be parked below the aortic arch 3006 and the procedure catheter 2904, access catheter 2902 (positioned within the procedure catheter 2904 and not visible in FIG. 9B), and a guidewire 2907 can be driven into the ostium (e.g., simultaneously or separately). In some embodiments, the access catheter 2902 is advanced out of the procedure catheter 2904 and the guide catheter 2906 to engage the ostium first. After the distal end of the access catheter 2902 is positioned within the desired ostium, the guidewire 2907 can be advanced distally into the ostium to secure access. After the access catheter 2902 and guidewire 2907 are positioned within the desired ostium, the procedure catheter 2904 and/or guide catheter 2906 can be advanced into the ostium (and, in some embodiments, beyond), while using the support of the access catheter 2902 and/or guidewire 2907 to maneuver through the aorta and into the ostium. In the embodiment shown in FIG. 9B, the procedure catheter 2904 has been advanced into the ostium while the guide catheter 2906 has remained parked below the aortic arch 3006.

Referring to FIG. 9C, the guidewire 2907 may be distally advanced and the radiopacity of the guidewire 2907 may be used to confirm under fluoroscopic imaging that access through the desired ostia has been attained. The guidewire 2907 engages the origin of the brachiocephalic artery 3014. The guidewire 2907 is then advanced up to the petrous segment 3018 of the internal carotid artery 3016.

Referring to FIG. 9D, the guide catheter 2906 and the procedure catheter 2904 (positioned within the guide catheter 2906 and not visible in FIG. 9D) are both advanced (e.g., simultaneously or sequentially) over the guidewire 2907 and over the insert or access catheter 2902 (positioned within the procedure catheter 2904 and not visible in FIG. 9D) while the access catheter 2902 remains at the ostium for support. The guidewire 2907 may be further advanced past the petrous segment 3018 to the site of the clot 3020, such as the M1 segment.

Referring to FIG. 9E, the guide catheter 2906 and the procedure catheter 2904 (positioned within the guide catheter 2906 and not visible in FIG. 9E) are advanced (e.g., simultaneously or sequentially) to position the distal tip of the procedure catheter 2904 at the procedure site, for example on the face of the clot 3020. The guidewire 2907 and access catheter 2902 (positioned within the procedure catheter 2904 and not visible in FIG. 9E) are removed, and aspiration of the clot 3020 commences through the procedure catheter 2904. That is, the guidewire 2907 and the access catheter 2902 are proximally retracted to allow aspiration through the procedure catheter 2904. After aspiration of the clot, the procedure catheter 2904 and guide catheter 2906 can be removed (e.g., simultaneously or sequentially). For example, in some embodiments, the procedure catheter 2904 may be removed before removing the guide catheter 2906.

In some embodiments, aspiration may be performed through two catheters (e.g., the procedure catheter 2904 and the guide catheter 2906) simultaneously. For example, during a thrombectomy procedure, the clot 3020 may become engaged with or corked at a distal end of the procedure catheter 2904 (or another inner catheter). In such cases, it may be necessary to remove the procedure catheter 2904 (or other inner catheter) from the vasculature of the patient to remove the clot 3020. As the procedure catheter 2904 (or other inner catheter) is retracted from the guide catheter 2906 (or other outer catheter), debris from the stuck clot 3020 can dislodge. In such embodiments, application of vacuum at both the procedure catheter 2904 (or other inner catheter) and the guide catheter 2906 (or other outer catheter) can beneficially prevent the debris from flowing distally into the vasculature of the patient by aspirating the debris and thus reducing the risk of embolization.

In some embodiments, the clot 3020 may become engaged with or corked at the distal end of the guide catheter 2906 (or other outer catheter). Vacuum through the guide catheter 2906 (or other outer catheter) may prevent dislodgement of the clot 3020 from the guide catheter 2906 (or other outer catheter). In some instances, it may not be readily apparent if portions of a clot are engaged with the guide catheter 2906 (or other outer catheter) or the procedure catheter 2904 (or other inner catheter). In such instances, vacuum through both the guide catheter 2906 (or other outer catheter) and the procedure catheter 2904 (or other inner catheter) may prevent debris from flowing distally into the vasculature of the patient by aspirating the debris and thus reducing the risk of embolization.

The multi-device assembly 2900 may be used to perform a neurovascular procedure, as described in FIGS. 9A-9E. For example, the neurovascular procedure may be a neurovascular thrombectomy. The steps of the procedure may include providing an assembly that includes at least a guidewire, an access catheter, a guide catheter, and a procedure catheter. For example, the multi-device assembly 2900 includes a guidewire 2907, an access (e.g., insert) catheter 2902, a guide catheter 2906, and at least one procedure catheter 2904. The procedure catheter 2904 may include an aspiration catheter, an embolic deployment catheter, a stent deployment catheter, a flow diverter deployment catheter, a diagnostic angiographic catheter, a stent retriever catheter, a clot retriever catheter, a balloon catheter, a catheter to facilitate percutaneous valve repair or replacement, an ablation catheter, and/or an RF ablation catheter or guidewire.

The neurovascular procedure may further include steps of coupling the assembly to a non-robotic or a robotic drive system, and driving the assembly to achieve supra-aortic access. The steps may further include driving a subset of the assembly to a neurovascular site, and performing the neurovascular procedure using a subset of the assembly. The subset of the assembly may include the guidewire, the guide catheter, and the procedure catheter.

Each of the guidewire 2907, the access catheter 2902, the guide catheter 2906, and the procedure catheter 2904 is configured to be adjusted by a respective hub. For example, the guidewire 2907 may include (or be coupled to) a hub installed on one of the tray assemblies described herein. Similarly, the access catheter 2902 may be coupled to insert or access catheter hub 2910. The guide catheter 2906 may be coupled to the guide catheter hub 2914. The procedure catheter 2904 may be coupled to the procedure catheter hub 2912.

In general, coupling of the assembly may include magnetically coupling a first hub 2909 on the guidewire 2907 to a first drive magnet, magnetically coupling a second hub 2910 on the access catheter 2902 to a second drive magnet, magnetically coupling a third hub 2912 on the procedure catheter 2904 to a third drive magnet, and magnetically coupling a fourth hub 2914 on the guide catheter 2906 to a fourth drive magnet. In general, the first drive magnet, the second drive magnet, the third drive magnet, and the fourth drive magnet are each independently movably carried by a drive table, as described with respect to tray assemblies and controls described herein. In some embodiments, the first drive magnet, the second drive magnet, the third drive magnet, and the fourth drive magnet are coupled (e.g., to their respective catheter hubs) through a sterile barrier (e.g., a sterile and fluid barrier) and independently movably carried by a drive table having a plurality of driven magnets. In some embodiments, two or more drive magnets can be tethered or otherwise coupled together to move as a unit in response to commands from a single controller tethered or otherwise coupled to one of the drive magnets.

In some implementations, the steps of performing the neurovascular procedure may include driving the assembly in response to movement of each of the hub adapters along a support table until the assembly is positioned to achieve supra-aortic vessel access. The hub adapters may include, for example, a coupler/carriage that acts as a shuttle by advancing proximally or distally along a track in response to operator instructions. The hub adapters described herein may each include at least one drive magnet configured to couple with a driven magnet carried by the respective hub. This provides a magnetic coupling between the drive magnet and driven magnet through the sterile barrier such that the respective hub is moved across the top of the sterile barrier in response to movement of the hub adapter outside of the sterile field (as described in detail in FIG. 4). Movement of the hub adapter is driven by a drive system carried by the support table in which the guidewire hub 2909, the guide catheter hub 2914, the procedure catheter hub 2912, and the insert or access catheter hub 2910 are installed upon.

The steps may further include driving a subset of the assembly in response to movement of each of the hub adapters along the support table until the subset of the assembly is positioned to perform a neurovascular procedure at a neurovascular treatment site. The subset of the assembly may include the guidewire 2907, the guide catheter 2906, and the procedure catheter 2904.

In some embodiments, the guidewire 2907, the guide catheter 2906 and the procedure catheter 2904 are advanced as a unit through (with respect to the guidewire 2907) and over (with respect to the guide catheter 2906 and the procedure catheter 2904) at least a portion of a length of the access (e.g., insert) catheter 2902 after supra-aortic access is achieved.

In some embodiments, the multi-device assembly 2900 may be part of a robotic control system for achieving supra-aortic access and neurovascular treatment site access, as described in FIGS. 9A-9E. In some embodiments, the multi-device assembly 2900 may be part of a manual control system for achieving supra-aortic access and neurovascular treatment site access. In some embodiments, the multi-device assembly 2900 may be part of a hybrid control system (with manual and robotic components) for achieving supra-aortic access and neurovascular treatment site access. For example, in such hybrid systems, supra-aortic access may be robotically driven while neurovascular site access and embolectomy or other procedures may be manual. Alternatively, in such hybrid systems, supra-aortic access may be manual while neurovascular site access may be robotically achieved. Still further, in such hybrid systems, any one or more of: the guidewire, access catheter, guide catheter, or procedure catheter may be robotically driven or manually manipulated.

An example robotic control system may include at least a guidewire hub (e.g., guidewire hub 2909) configured to adjust each of an axial position and a rotational position of a guidewire 2907. The robotic control system may also include an insert or access catheter hub 2910 configured to adjust axial and rotational movement of an access catheter 2902. The robotic control system may also include a guide catheter hub 2914 configured to control axial movement of a guide catheter 2906. The robotic control system may also include a procedure catheter hub 2912 configured to adjust an axial position and a rotational position of a procedure catheter 2904.

In some embodiments, the procedure catheter hub 2912 is further configured to laterally deflect a distal deflection zone of the procedure catheter 2904.

In some embodiments, the guidewire hub 2909 is configured to couple to a guidewire hub adapter by magnetically coupling the guidewire hub to a first drive magnet. The insert or access catheter hub 2910 is configured to couple to an access catheter hub adapter by magnetically coupling the insert or access catheter hub 2910 to a second drive magnet. The procedure catheter hub 2912 is configured to couple to a procedure catheter hub adapter by magnetically coupling the procedure catheter hub 2912 to a third drive magnet. The guide catheter hub 2914 is configured to couple to a guide catheter hub adapter by magnetically coupling the guide catheter hub 2914 to a fourth drive magnet. In some embodiments, the first drive magnet, the second drive magnet, the third drive magnet, and the fourth drive magnet are independently movably carried by a drive table.

In some embodiments, the robotic control system includes a first driven magnet on the guidewire hub 2909. The first driven magnet may be configured to cooperate with the first drive magnet such that the first driven magnet is configured to move in response to movement of the first drive magnet. In some embodiments, the first drive magnet is configured to move outside of a sterile field separated from the first driven magnet by a barrier while the first driven magnet is within the sterile field. In some embodiments, a position of the first driven magnet is movable in response to manipulation of a procedure drive control on a control console associated with the drive table. Drive magnets and driven magnet interactions are described in detail with respect to FIG. 4 above.

In some embodiments, the robotic control system includes a second driven magnet on the insert or access catheter hub 2910. The second driven magnet may be configured to cooperate with the second drive magnet such that the second driven magnet is configured to move in response to movement of the second drive magnet. In some embodiments, the second drive magnet is configured to move outside of a sterile field separated from the second driven magnet by a barrier while the second driven magnet is within the sterile field.

In some embodiments, the robotic control system includes a third driven magnet on the procedure catheter hub 2912. The third driven magnet may be configured to cooperate with the third drive magnet such that the third driven magnet is configured to move in response to movement of the third drive magnet. In some embodiments, the third drive magnet is configured to move outside of a sterile field separated from the third driven magnet by a barrier while the third driven magnet is within the sterile field.

In some embodiments, the robotic control system includes a fourth driven magnet on the guide catheter hub 2914. The fourth driven magnet may be configured to cooperate with the fourth drive magnet such that the fourth driven magnet is configured to move in response to movement of the fourth drive magnet. In some embodiments, the fourth drive magnet is configured to move outside of a sterile field separated from the fourth driven magnet by a barrier while the fourth driven magnet is within the sterile field. In some embodiments, there may be more than four driven magnets and corresponding catheter hubs for control of additional catheters.

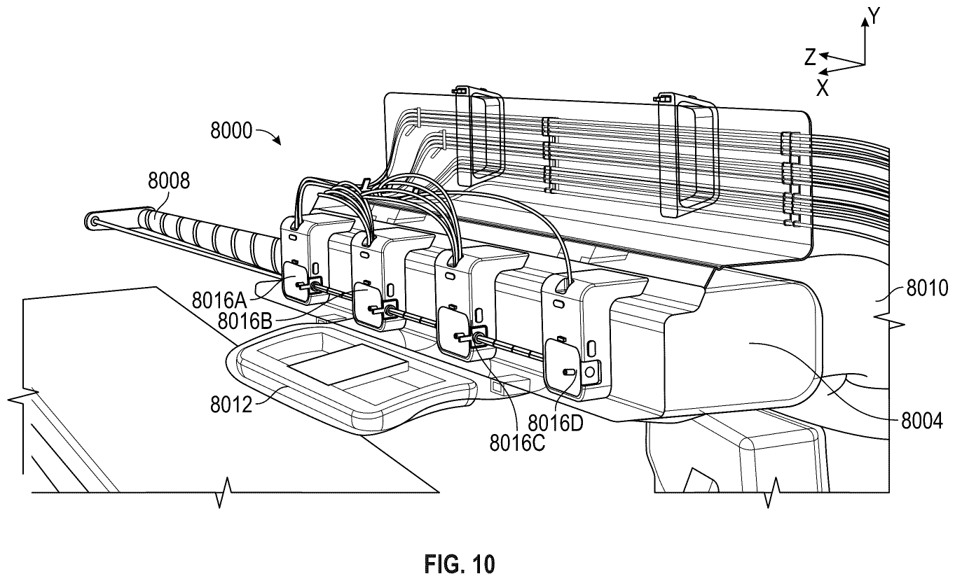

FIG. 10 illustrates an example of a drive table 8000. The drive table 8000 can include any of the same and/or similar features as any of the drive tables described herein and vice versa. The drive table 8000 can include a main body 8004 (which may also be referred to as a shuttle). In some embodiments, the drive table 8000 can include one or more planar support surfaces 8006A-B. In some embodiments, the drive table 8000 can include an extendable or telescoping member 8008 (also referred to herein as a hockey stick, a telescoping arm, or a telescoping support). The drive table 8000 can be further coupled to a base 8010. In some embodiments, the drive table 8000 can further include a handle 8012. The drive table 8000 can be configured to position a drive assembly proximal to an access point on a patient.

The drive table 8000 can include a plurality of hub assemblies. The plurality of hub assemblies can include a first hub assembly 8016A, a second hub assembly 8016B, a third hub assembly 8016C, and/or a fourth hub assembly 8016D. Each of the plurality of hub assemblies can be configured to translate along the one or more planar support surfaces 8006A-B of the drive table 8000 or a drive surface placed over one or more of the planar support surfaces 8006A-B of the drive table (e.g., a drive surface of a sterile barrier placed over one or more of the planar support surfaces 8006A-B). In some embodiments, the plurality of hub assemblies can be magnetically coupled to a corresponding carriage or hub adapter of the drive system located within the main body 8004. Each hub assembly of the plurality of hub assemblies can move relative to the main body 8004. For example, each hub assembly of the plurality of hub assemblies can be moved in a distal direction thereby advancing the plurality of hub assemblies and their corresponding interventional devices into the patient. Each hub assembly of the plurality of hub assemblies can be moved in a proximal direction thereby retracting the plurality of hub assemblies and their corresponding interventional devices away from the patient.

The telescoping arm or telescoping member 8008 can be a longitudinal body configured to extend from and/or retract into the main body 8004. In some embodiments, the telescoping member 8008 can be a support bracket. In some embodiments, the telescoping member 8008 may be configured to support one or more interventional devices at a position proximal to a patient. In some embodiments, the telescoping member 8008 can be coupled to an access sheath (e.g., a femoral access sheath) at a patient access point. In some cases, the main body 8004 can be advanced and/or retracted over the telescoping member 8008. This can allow the position of the main body 8004 relative to a patient to be adjusted. For example, the main body 8004 can be advanced over the telescoping member 8008 to bring the main body 8004 closer to the patient thereby advancing the plurality of hub assemblies and their corresponding interventional devices into the patient. The main body 8004 can be retracted over the telescoping member 8008 to bring the main body 8004 away from the patient thereby retracting the plurality of hub assemblies and their corresponding interventional devices from the patient. Additional details regarding drive tables are disclosed in U.S. application Ser. No. 19/228,455, entitled DRIVE TABLE, filed Jun. 4, 2025, the entirety of which is hereby expressly incorporated by reference herein. Additional details regarding drive tables are disclosed in U.S. patent application Ser. No. 18/524,879, entitled “ROTATABLE DRIVE TABLE,” filed Nov. 30, 2023, the entirety of each of which is hereby expressly incorporated by reference herein.

In some embodiments, the arm or member 8008 may not telescope within the main body 8004. The body 8004 may move relative to the arm or member 8008 in an alternative manner. For example, the arm or member 8008 may include a track positioned exterior to the main body 8004 along which the main body 8004 can translate.

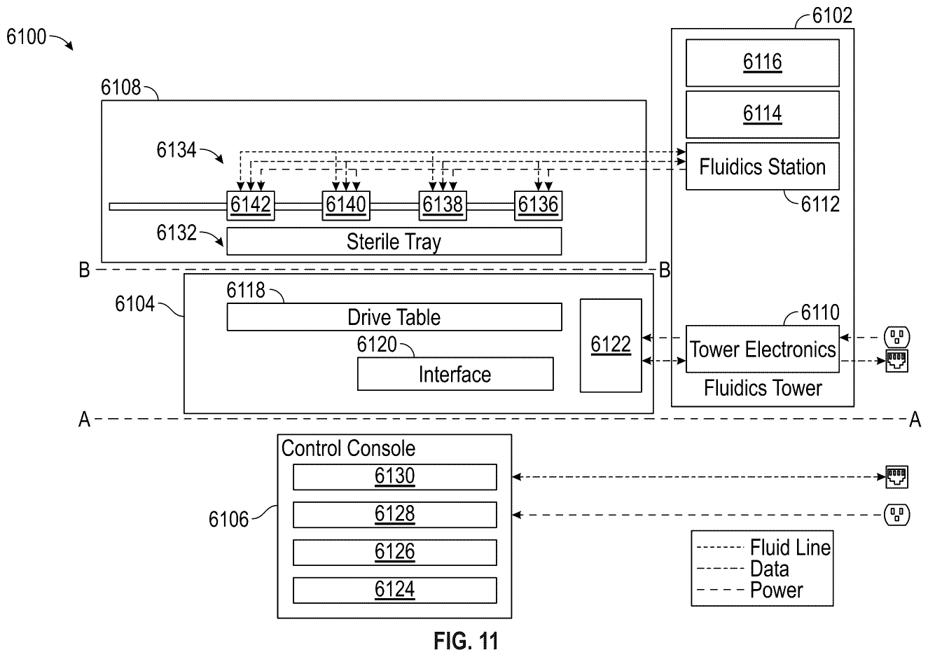

FIG. 11 illustrates a system diagram of an example of a medical device operation system 6100. The system diagram shows fluid and electrical connectivity between the subsystems of the medical device operation system 6100. The medical device operation system 6100 may include a fluidics tower 6102, a robotic drive system 6104, a control system 6106, and one or more interventional devices 6108.

The fluidics tower 6102 may be a housing or console including a fluidics management system for controlling the administration or removal of contrast, saline and/or bodily fluids to and/or from an interventional device. The fluidics tower 6102 may further include an electronics tower 6110, a fluidics station (or “system”) 6112, a monitor 6114, and one or more communication devices 6116. Although illustrated in FIG. 11 as part of the fluidics tower 6102, in other embodiments the electronics tower 6110 may be housed separately from the fluidics tower 6102 while still being in communication with the fluidics tower 6102, the interventional devices 6108, the robotic drive system 6104, and the control console system.

The electronics tower 6110 may be a housing configured to contain system electronics such as one or more processors and memory. The one or more processors and memory may be organized into one or more computer devices. The electronics tower 6110 may include a power cord configured to be operatively coupled to a power source, such as a battery, a generator, or an outlet. In some embodiments, the electronics tower 6110 may draw power from a source providing 110/220 volts of alternating current (VAC) power.

The system electronics may be a central hub for the medical device operation system 6100, interconnecting the electronic devices from other components as described in greater detail below. The system electronics of the electronics tower 6110 may be configured to transmit and receive electronic signals and/or data to operate components of the medical device operation system 6100. The electronics tower 6110 may include means for connecting to other devices. The electronics tower 6110 may transmit and/or receive electronic signals and/or data wirelessly or over a wired connection. In some embodiments, the electronics tower 6110 may include an Ethernet port for connecting the system electronics to a network. In some embodiments, the electronics tower 6110 may include one or more ports for tethering to nearby electronic devices via a wired connection. For example, the electronics tower 6110 may have ports to run cables between the fluidics tower 6102 and the robotic drive system 6104 and/or control system 6106. Alternatively, the electronics tower 6110 may be configured to connect wirelessly to nearby electronic devices. For example, the electronics tower 6110 may include a personal area network (PAN) module, such as Bluetooth®, or other network capabilities to transmit data wirelessly.

In some embodiments, electronic signals and/or data may include instructions for a system, subsystem, component, or device to perform a particular task. Additionally and/or alternatively, electronic signals and/or data may include indicators or data measured from sensors for processing by a computing device.

In some embodiments, the electronics tower 6110 may connect to other devices over a communication network (“network”). The network may cover a small geographic area, such as a particular room or building, a medium geographic area, such as a city, or a large geographic area, so long as there is access to a network. For example, the network may be a local area network (LAN) or wireless local area network (WLAN) including a series of devices linked together to form a network within a hospital or clinic. Alternatively, the network may be a wide-area network (WAN) including a series of devices linked together to form a network within a medical campus including two or more buildings. Alternatively, the network may be an intranet or internet for providing global connectivity. Connecting over the network advantageously connects the operating room to physicians located around the world, including experts located across the nation or in other countries, without requiring the physician to travel to the operating room. This advantageously connects patients to physicians without the time or cost required for the physician to physically travel to the operating room. In some procedures, every minute of delay before a procedure is performed can increase the chance of a bad outcome, and thus such surgical systems can help mitigate damage to the patient due to a delay in starting the surgical procedure.

The fluidics system 6112 may include one or more subsystems including one or more containers, one or more tubes, and one or more pumps. The subsystems may be divided and organized into a contrast subsystem, a saline subsystem, and/or an aspiration (or “vacuum”) subsystem. The fluidics system 6112 may be the fluidics system described herein.

A contrast subsystem may be configured for supplying contrast to a patient. The contrast subsystem may include one or more containers for storing and supplying contrast, one or more fluid communication channels (“tubes”), one or more valves, and a high-pressure pump.

A saline subsystem may be configured for supplying saline to a patient. The saline subsystem may similarly include one or more containers for storing and supplying saline, one or more tubes, one or more valves, and one or more pumps.

An aspiration subsystem may be configured for removing biological material from a patient. The aspiration subsystem may include one or more containers, one or more tubes, one or more valves, and a vacuum pump.

The one or more pumps and containers of the contrast, saline, and aspiration subsystems may be contained within the fluidics tower 6102. The one or more tubes of the contrast, saline, and aspiration subsystems may extend out of the fluidics tower 6102 for interacting with and coupling to other devices.

The monitor 6114 may be any electronic visual computer display (or displays) that includes a screen and circuitry configured to interpret electronic signals to display one or more images. For example, the monitor 6114 may include an imaging window, a speed indicator, a rotational indicator, an axial position bar, a telescopic position window, one or more axial position indicators, and/or other graphical user interfaces or windows. In some embodiments, the monitor 6114 may be configured to display fluoroscopic images, catheter data, fluidics information (e.g., information relating to a contrast injection subsystem, including its current operation status, information relating to a saline subsystem, including its current operation status, and/or information relating to an aspiration subsystem, including its current operation status), and patient data including vital signs. In some embodiments, the monitor 6114 may be the display 23 described above.

The one or more communication devices 6116 may be one or more microphones, one or more cameras, and/or one or more audio output devices such as a speaker and/or a headset.

The fluidics system 6112, the monitor 6114, and the one or more communication devices 6116 may be in electrical communication with the electronics tower 6110 and configured to receive and/or transmit electronic signals and/or data therebetween. In some embodiments, the electronic signals and/or data may include instructions to activate one or more pumps and/or one or more valves of the fluidics system 6112. For example, the instructions may direct the fluidics system 6112 to provide saline and/or contrast to the one or more interventional devices 6108. In some embodiments, the data may include video and/or audio inputs and audio outputs for the monitor 6114 and one or more communication devices 6116. For example, the data may be one or more images to be displayed on the monitor 6114 and/or audio-visual data captured by the one or more communication devices 6116.

The fluidics tower 6102 may be further configured to be operatively coupled with the one or more interventional devices 6108. In some embodiments, the fluidics system 6112 may be mechanically coupled to and/or in fluid communication with the one or more interventional hubs 6134. Accordingly, activating the fluidics system 6112 may provide contrast, saline, and/or suction to the interventional hubs 6134 and corresponding interventional devices.

The robotic drive system 6104 may include a plurality of components to drive one or more access systems, such as catheters and guidewires, during a procedure. The robotic drive system 6104 may be the drive system 18 described above. The robotic drive system 6104 may include a drive table 6118, an interface 6120, and a joint setup 6122.

The drive table 6118 may support the one or more disposable devices 6108 (e.g., a catheter) configured to be advanced to access a patient for performing a surgical procedure and/or for introducing saline, contrast media, or therapeutic agents, or providing aspiration. The drive table 6118 may further support a sterile barrier.

The drive table 6118 may be the support table 20 described above. The drive table 6118 may be positioned over or alongside a patient, and configured to axially advance, retract, and in some cases rotate two or three or more different concentrically oriented intravascular devices of an interventional device assembly. The drive table 6118 may include electronics and motors for controlling the location of the interventional devices and actuation of fluidics components.

In some embodiments, the drive table 6118 may include one or more hub adapters. The one or more hub adapters may include the drive magnets described above. Movement of the drive magnets may be driven by a drive system carried by the drive table 6118. Movement of the drive magnets may be configured to drive one or more interventional hubs 6134 of the one or more disposable devices 6108. The drive table 6118 and the one or more disposable devices 6108 may be separated such that the one or more interventional hubs 6134 may not mechanically couple to the drive table 6118, as shown by axis B-B.

The interface 6120 may be any device configured to interact with and/or display information to personnel locally situated within an operating room during a procedure, such as a bedside user. For example, a bedside user may be a nurse or surgical technician staffed within the operating room. The interface 6120 may include an imaging window, a speed indicator, a rotational indicator, an axial position bar, a telescopic position window, and/or one or more axial position indicators. The interface 6120 may be the display 23 described above configured to display fluoroscopic images, catheter data, pressure values of the fluidics system, and/or other patient data. In some embodiments, the interface 6120 may be a touchscreen device such as a tablet computer. The interface 6120 may display information to a bedside user. The information displayed to the bedside user may include directions and/or prompts for the bedside user to follow. For example, the information may describe what steps to perform next, how to position the robotic drive system 6104, when to deploy the drapes, whether the system is malfunctioning or whether an error is detected, and/or prompt the bedside user to otherwise interact with the system. In some embodiments, the interface 6120 is configured to accept user input to control one or more components, for example, the position of the drive table.

The interface 6120 may be in communication with one or more portions of the medical device operation system 6100 (for example, the robotic drive system 6104, the fluidics tower 6102, the disposable devices 6108, etc.). In some embodiments, the interface 6120 may be mechanically coupled to the robotic drive system 6104, be housed separately, or be mechanically coupled to another part of the medical device operation system 6100. The interface 6120 may control the joint setup 6122 of the robotic drive system 6104. For example, the interface 6120 may control the transition processes between a storage position and a deployed position, engaging a priming sequence, or controlling fine motor adjustments for providing minor adjustments to the positions of the interventional hubs. Controlling the joint setup 6122 and motors with the interface 6120 advantageously provides greater precision and setup before an operation by individuals present in the operating room.

The interface 6120 may advantageously provide a backup control mechanism to interact with and provide input to control the medical device operation system 6100, for example, in the event that the control system 6106 is rendered incapable of performing an operation.

The joint setup 6122 may include a plurality of joints and motors for controlling the positioning and movements of the robotic drive system 6104. In some embodiments, the joint setup 6122 may initialize the robotic drive system 6104 into a starting position. The initialization process may include transitioning the robotic drive system 6104 from a storage position to a deployed position and vice versa. For example, the joint setup 6122 may be configured to transition the robotic drive system 6104 from a storage position of the robotic drive system 6104 to a deployed position such that at least a portion of the robotic drive system 6104 transitions from a compact state to a position where at least a portion of the robotic drive system 6104 is positioned either over or alongside a patient.

Within the robotic drive system 6104, the drive table 6118 may be mechanically coupled with the interface 6120 and the joint setup 6122. The joint setup 6122 may also be electrically connected to the drive table 6118 and the interface 6120. The robotic drive system 6104 may be configured for the joint setup 6122 to transmit electronic signals and data to the drive table 6118 and the interface 6120. Additionally and/or alternatively, the robotic drive system 6104 may be configured for the joint setup 6122 to receive electronic signals and data from the drive table 6118 and the interface 6120.

The control system 6106 may be a collection of components configured to control and operate the robotic control system described above. In some embodiments, the control system is a control console or is coupled to a control console. The control system 6106 may further include an operator controller 6124, an interface 6126, a monitor 6128, and one or more communication devices 6130. The control system 6106 may be locally positioned or remotely positioned. For example, in some embodiments, the control system 6106 may be located in the operating room with the fluidics tower 6102, the robotic drive system 6104, and the one or more disposable devices 6108. Alternatively, the control system 6106 may be located remotely (e.g., in a control room) as illustrated by line A-A. The control system 6106 may include system electronics including one or more processors and one or more memory components (“memory”). The system electronics may be configured to electrically connect the controller 6124, the interface 6126, the monitor 6128, and the one or more communication devices 6130.

The control system 6106 may include means for connecting to other devices. The control system 6106 may transmit and/or receive electronic signals and/or data wirelessly or over a wired connection. In some embodiments, the control system 6106 may include an Ethernet port for connecting the system electronics to a network. In some embodiments, the control system 6106 may include one or more ports for tethering to nearby electronic devices via a wired connection. For example, the control system 6106 may have ports to run cables between the control system 6106 and the fluidics tower 6102 and/or robotic drive system 6104. Alternatively, the control system 6106 may be configured to connect wirelessly to nearby electronic devices. For example, the control system 6106 may include a Bluetooth® module or other network capabilities to transmit data wirelessly.

In some embodiments, the control system 6106 may connect to other devices over a network as described above.

The controller 6124 may be any device configured to enable a surgeon to control portions of the medical device operation system 6100 in the same location as the patient. For example, the controller 6124 may be any of the control mechanisms or controllers described herein. The controller 6124 may enable a user to control portions of the fluidics tower 6102, the interventional devices 6108, and the robotic drive system 6104. For example, the controller 6124 may be configured to move to desired positions to perform a procedure on a patient as described herein.

The controller 6124 may be part of the control system 6106 or connected, wirelessly or via a wired connection, to the control system 6106.

The interface 6126 may be configured to display information to the surgeon. The interface 6126 may be the display 23 described above configured to display fluoroscopic images, catheter data, or other patient data. The interface 6126 may be a touchscreen device. The interface 6126 may be a graphical user interface.

The monitor 6128 may include one or more electronic displays. The monitor 6128 may be any electronic visual computer display that includes a screen and circuitry configured to interpret electronic signals to display one or more images. The monitor may display the interface 6126. In some embodiments, the monitor 6128 may be configured to display fluoroscopic images, catheter data, or other patient data. Alternatively, the monitor 6128 may be configured to display one or more views of the operating room. For example, the monitor 6128 may be configured to display the working area during a procedure by displaying only the surgical site. In another example, the monitor 6128 may display the entire operating room including the surgical technicians. In another example, the monitor 6128 may display more than one view. Displaying a plurality of views to capture the entire operating room may advantageously enhance communication and understanding between the physician and the technicians and/or assistants located in the operating room thereby increasing the efficiency and safety of procedures.

The one or more communication devices 6130 may be any one or more microphones, one or more cameras, and/or one or more audio output devices.

As shown in FIG. 11, the fluidics tower 6102, the robotic drive system 6104, the control system 6106, and the one or more disposable devices 6108 are connected to a power source. In some embodiments, the fluidics tower 6102 and the control system 6106 may be directly connected to a power source such as an outlet. In some embodiments, the robotic drive system and the one or more disposable devices 6108 may indirectly connect to a power source. For example, the robotic drive system 6104 and the one or more disposable devices 6108 may receive power from the fluidics tower 6102. In such embodiments, the joint setup 6122 of the robotic drive system 6104 may be electrically connected to the electronics tower 6110 of the fluidics tower and the interventional hubs 6134 of the one or more disposable devices 6108 may be electrically connected to the fluidics system 6112 of the fluidics tower 6102 such that power may be transmitted therebetween.

Furthermore, as shown in FIG. 11, the fluidics tower 6102, the robotic drive system 6104, the control system 6106, and the one or more disposable devices 6108 may be electrically connected and configured to share electrical signals and/or data. In some embodiments, the fluidics tower 6102 may be electrically connected and configured to share electrical signals and/or data with the robotic drive system 6104, the control system 6106, and the one or more disposable devices 6108. For example, the electronics tower 6110 of the fluidics tower 6102 may be electrically connected with the joint setup 6122 of the robotic drive system 6104 and the control system 6106 while the fluidics system 6112 of the fluidics tower 6102 may be electrically connected with the one or more interventional hubs 6134 of the one or more disposable devices 6108. In some embodiments, the electronics tower 6110 may be electrically connected with the control system 6106 via a network, as shown in FIG. 11.

The one or more interventional hubs 6134 may include a first interventional hub 6136, a second interventional hub 6138, a third interventional hub 6140, and a fourth interventional hub 6142. In some embodiments, the one or more interventional hubs 6134 may be aligned sequentially such that the first interventional hub 6136 may be positioned at a first end and the fourth interventional hub 6142 may be positioned at a second end opposite the first end. In some embodiments, the first end may be a proximal end closest to a patient and the second end may be a distal end furthest from the patient. A sterile tray 6132 may separate the one or more interventional hubs 6134 and corresponding interventional devices from a support table. In some embodiments, the sterile tray 6132 forms a sterile barrier, such as the sterile barrier 32 described above.

In some embodiments, the first interventional hub 6136 may be a guidewire hub, such as the guidewire hub 26 described above; the second interventional hub 6138 may be a first catheter hub, such as the access catheter hub 2910 described above; the third interventional hub 6140 may be a second catheter hub configured to engage with and guide a procedure catheter, such as the procedure catheter hub 2912 described above; and the fourth interventional hub 6142 may be a third catheter hub configured to engage with and guide a guide catheter, such as the guide catheter hub 2914. In some embodiments, the guide catheter may extend distally from the fourth interventional hub 6142.

The fluidics tower 6102 may be electrically connected to the robotic drive system 6104, the control system 6106, and the one or more disposable devices 6108, wherein electrical signals and/or data may be transmitted between therebetween as discussed in greater detail below. The local system may transmit information about the fluidics system 6112, the robotic drive system 6104, and the plurality of interventional devices 6108 to the control system 6106 via the fluidics tower 6102.

The one or more communication devices 6130 may be in electrical communication with a power source. The one or more communication devices 6130 may further be in electrical communication with the electronics tower 6110 and configured to receive and/or transmit electronic signals and/or data therebetween. In some embodiments, the one or more communication devices 6130 are in electrical communication with the electronics tower 6110 via a network. For example, the one or more communication devices 6130 may be electrically coupled to an ethernet cable configured to connect the one or more communication devices 6130 to a network, wherein the electronics tower 6110 may be electrically coupled to the network.

In some embodiments, devices (e.g., hubs, hub adapters, interventional devices, and/or trays) described herein may be used during a robotically driven procedure. For example, in a robotically driven procedure, one or more of the interventional devices may be driven through vasculature and to a procedure site. Robotically driving such devices may include engaging electromechanical components that are controlled by user input. In some implementations, users may provide the input at a control system that interfaces with one or more hubs and hub adapters.

In some embodiments, the hubs, hub adapters, interventional devices, and trays described herein may be used during a non-robotic (e.g., manually driven) procedure. Manually driving such devices may include engaging manually with the hubs to affect movement of the interventional devices.

In some embodiments, the devices described herein may be used to carry out a method of performing an intracranial procedure at an intracranial site. The method of performing the intracranial procedure may include any of the same steps as described herein for performing a neurovascular procedure. The procedure may be robotically performed, manually performed, or a hybridized combination of both.

While the foregoing describes magnetic coupling of hubs to drive magnets, in other embodiments, any of the interventional devices and/or hubs may be mechanically coupled to a drive system. Any of the methods described herein may include steps of mechanically coupling one or more interventional devices (e.g., the guidewire 2907, the access catheter 2902, the procedure catheter 2904, and/or the guide catheter 2906) and/or one or more hubs (e.g., the guidewire hub 2909, the insert or access catheter hub 2910, the procedure catheter hub 2912, and/or the guide catheter hub 2914) with one or more drive mechanisms.

### Treatment of Pulmonary Embolism

A pulmonary embolism (PE) is a blood clot that blocks blood flow to one or more pulmonary arteries. PE typically occurs when a blood clot starts somewhere else in the body, breaks off, and travels through the heart and into the pulmonary arteries. Accordingly, PE is commonly associated with deep vein thrombosis. PE causes restriction of nutrients and oxygen to the heart and an increase in right ventricle/right atrium (RV/RA) pressure. PE can often lead to heart failure and has a high reoccurrence rate. PE affects over 900,000 patients in the United States every year. Every year, over 100,000 patients die from PE. If left untreated, PE has a 30% mortality rate with a significantly decreased quality of life. When treated with timely therapies, PE has an 8% mortality rate. PE severity is classified as either massive, sub-massive, or low-risk. Low-risk patients often see symptoms and reduced quality of life. Low risk PE is normally only treated with anticoagulant and therapy. Massive and sub-massive PE patients require a heavier degree of therapy, usually surgical intervention paired with medication. Currently, thrombectomy is used to treat massive and sub-massive patients (61%). Quick treatment of PE is vital to minimize stress on the heart H and reduce the chance of heart H failure.

The present disclosure provides a robotically controlled multi-catheter stack for treatment of pulmonary embolism (PE). The robotically controlled systems and methods disclosed herein can decrease transfer time, increase access, and improve the timeliness of therapy, thereby reducing the PE mortality rate. Furthermore, the systems and methods described herein can reduce the total number of devices and device swaps required for PE treatment. Integration of a fluidics system can further enable patient information (e.g., heart pressure readings) to be read and relayed for remote use, leading to blood loss mitigation.

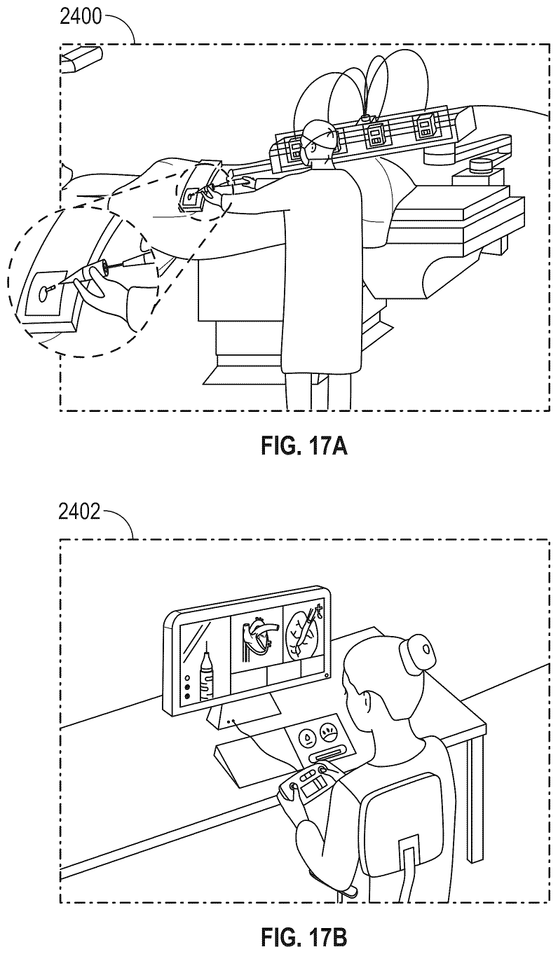

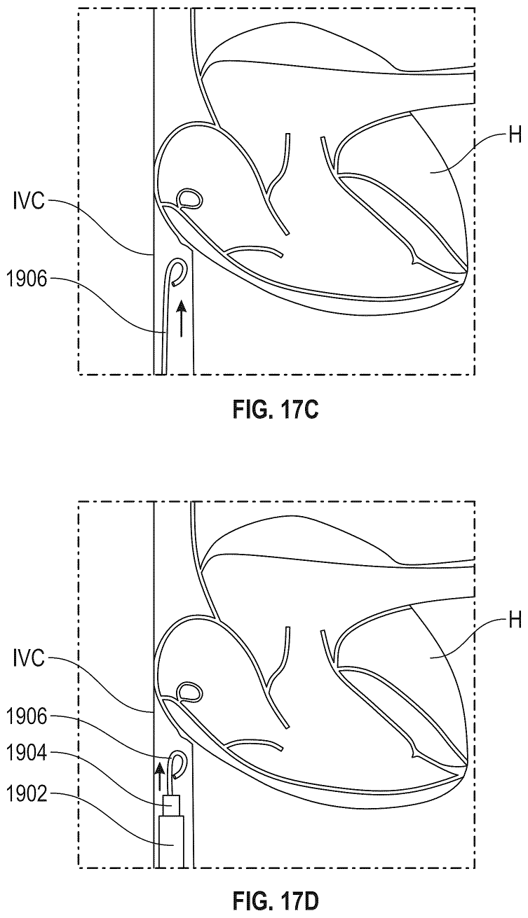

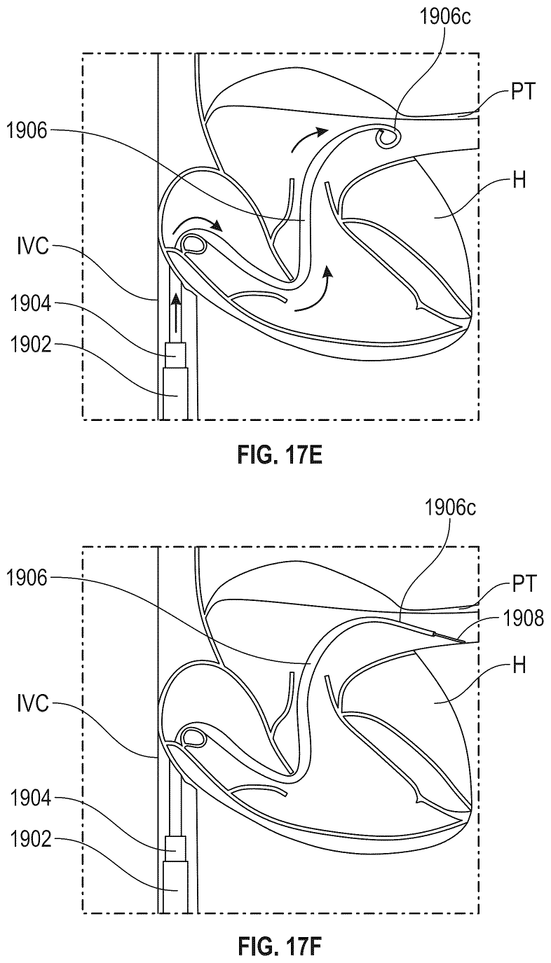

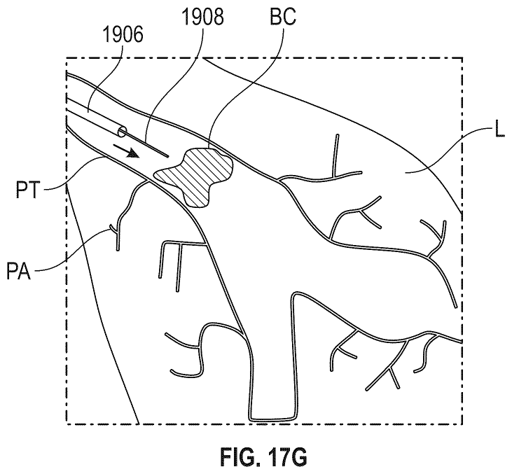

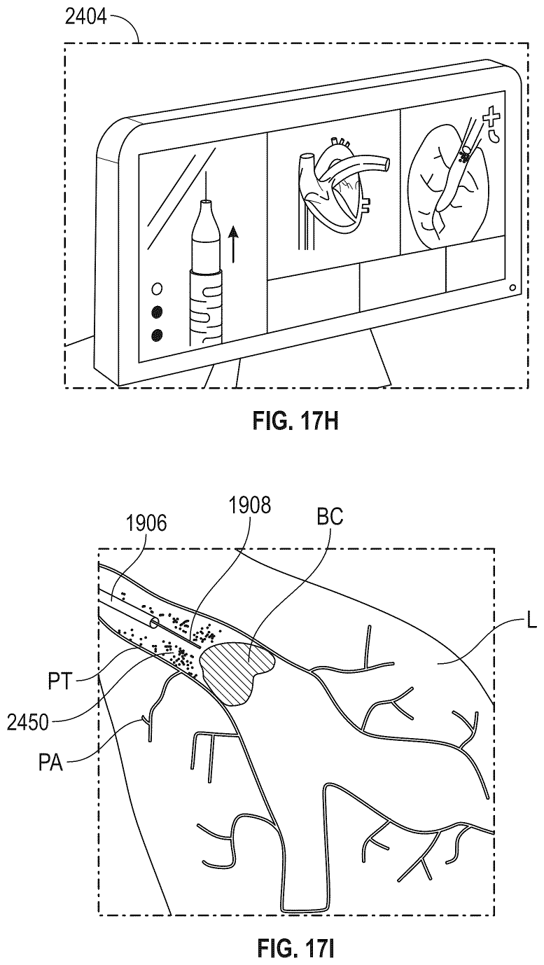

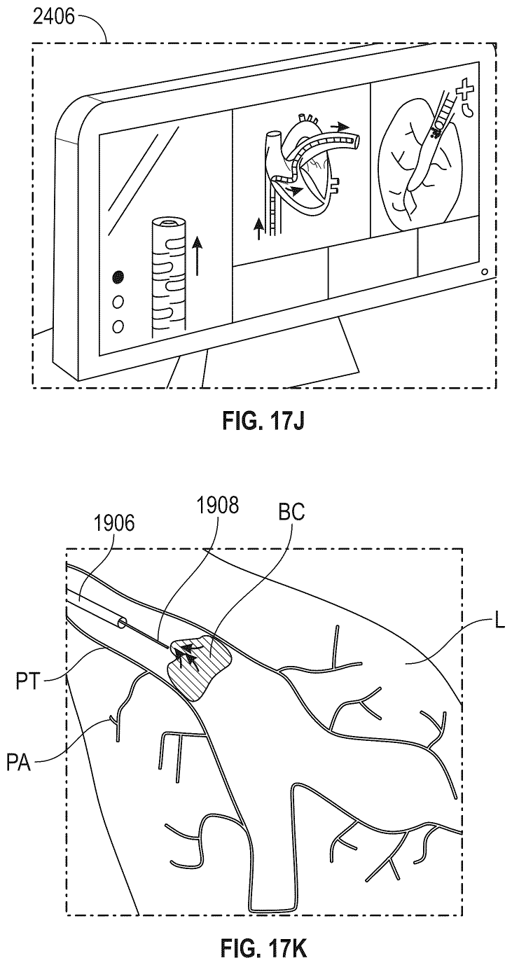

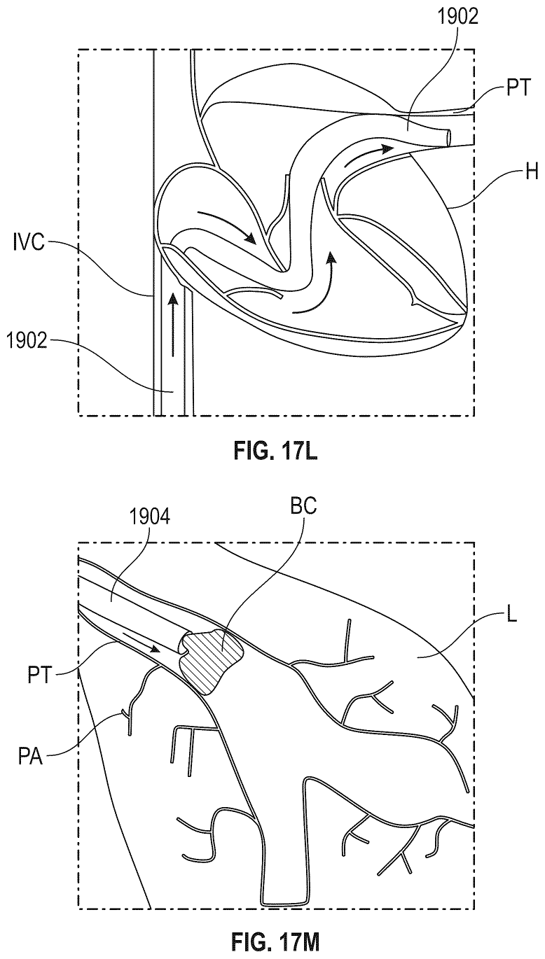

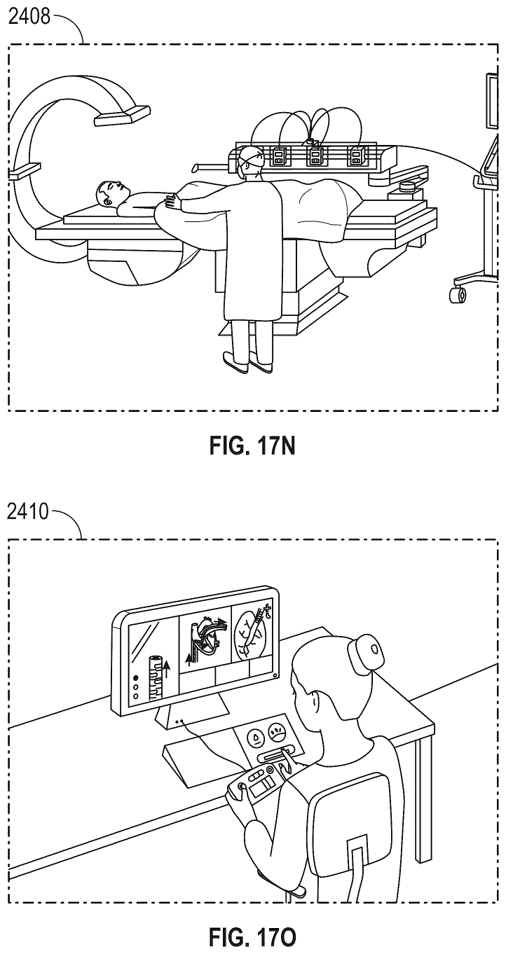

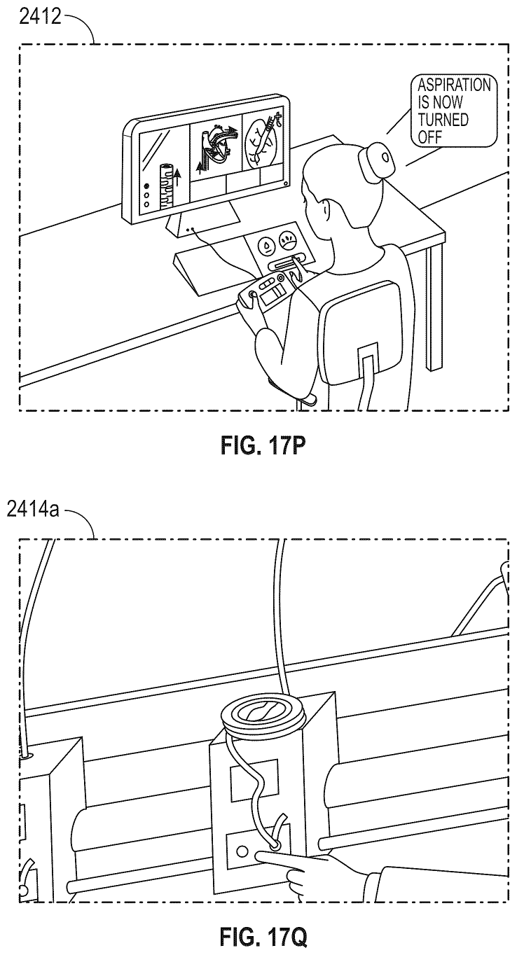

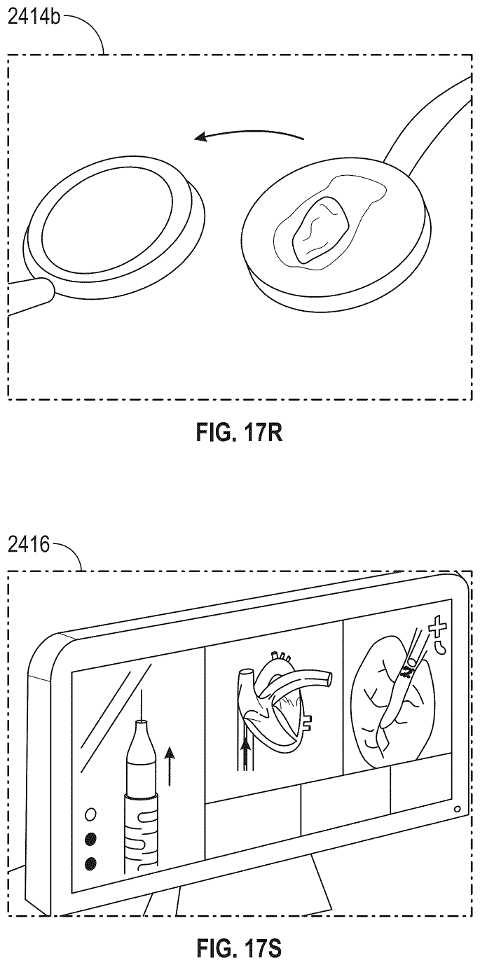

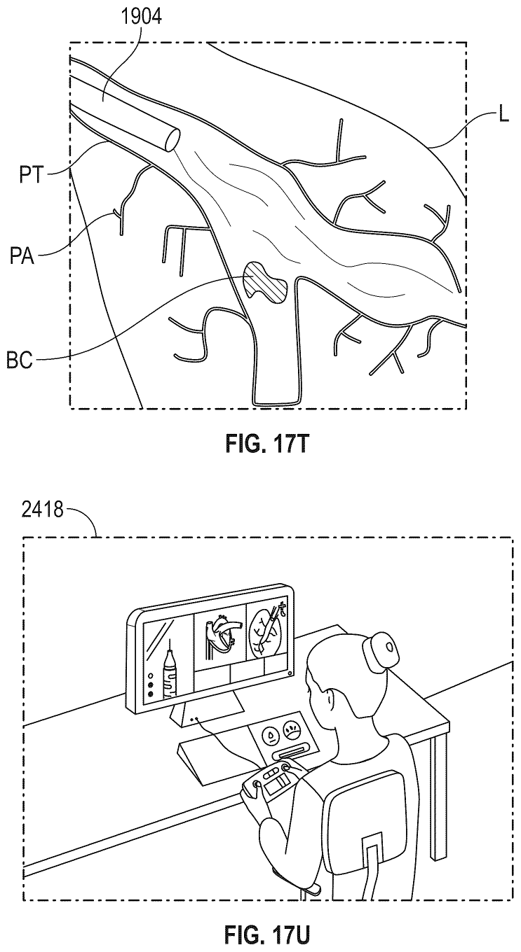

In some embodiments, the multi-device assembly 2900 of FIG. 8 can be utilized to treat pulmonary embolism. For example, the multi-device assembly 2900 can be utilized to treat pulmonary embolism according to the method described with respect to FIGS. 17A-17U in the same or similar manner as the multi-device stack 1900 can be utilized.

In some cases, the multi-device assembly 2900 can be inserted through an additional larger sheath (e.g., introducer sheath 3002). In some embodiments, the assembly 2900 can be introduced through a short sheath (e.g., shorter than the guide catheter 2906). In some embodiments, the additional sheath can be manually placed.

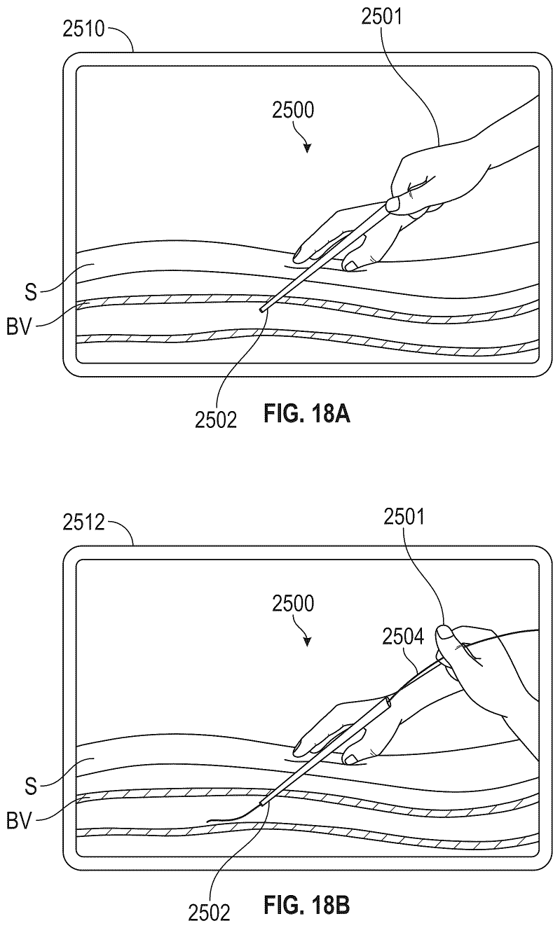

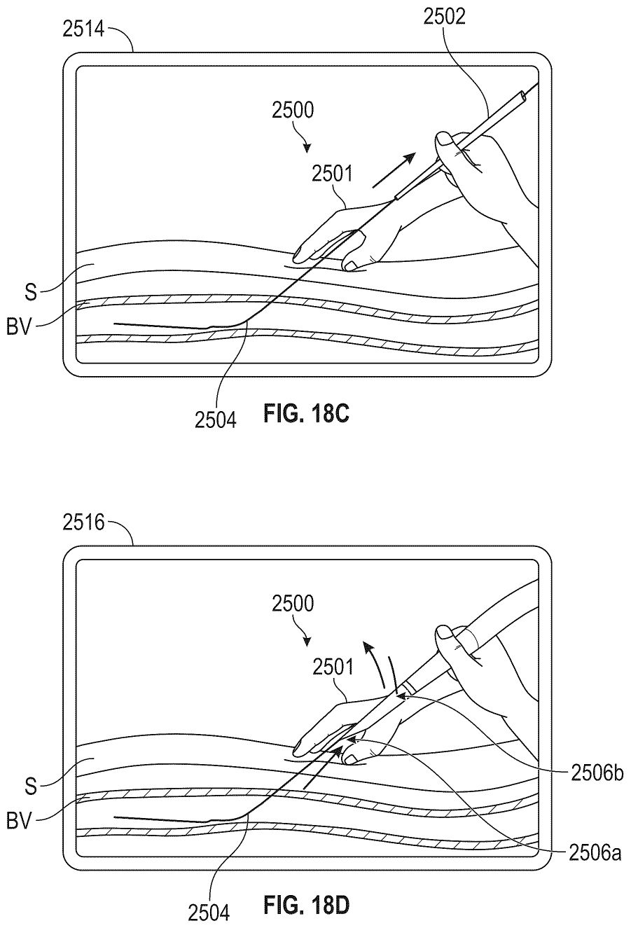

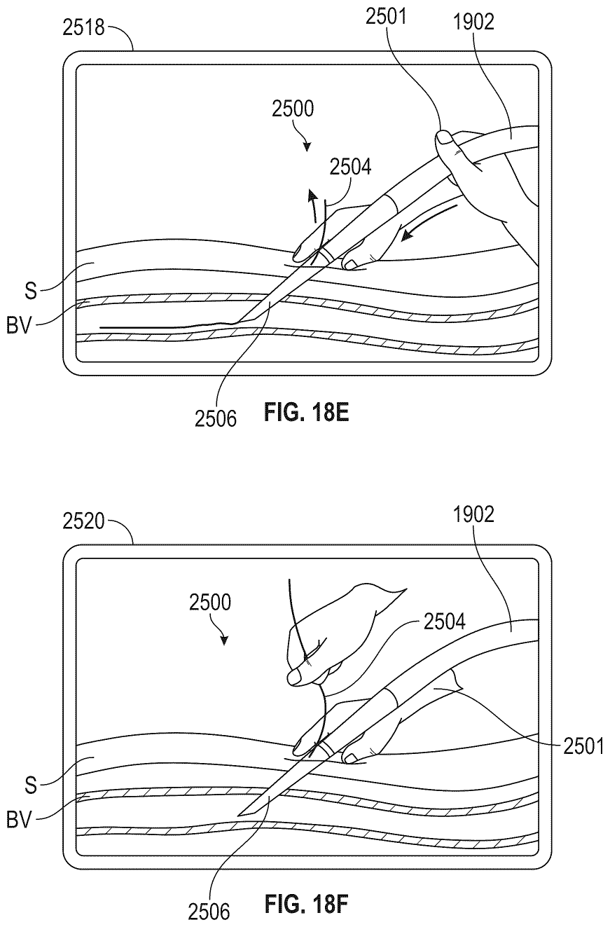

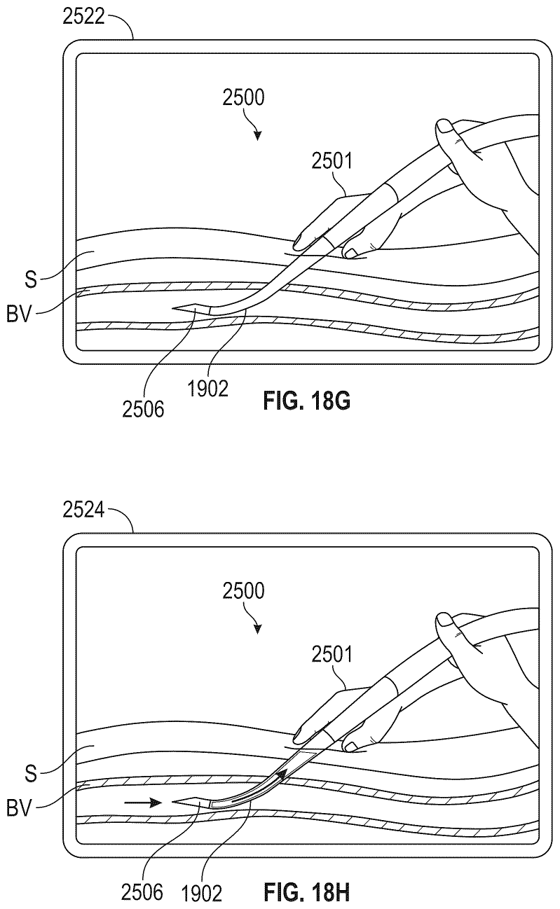

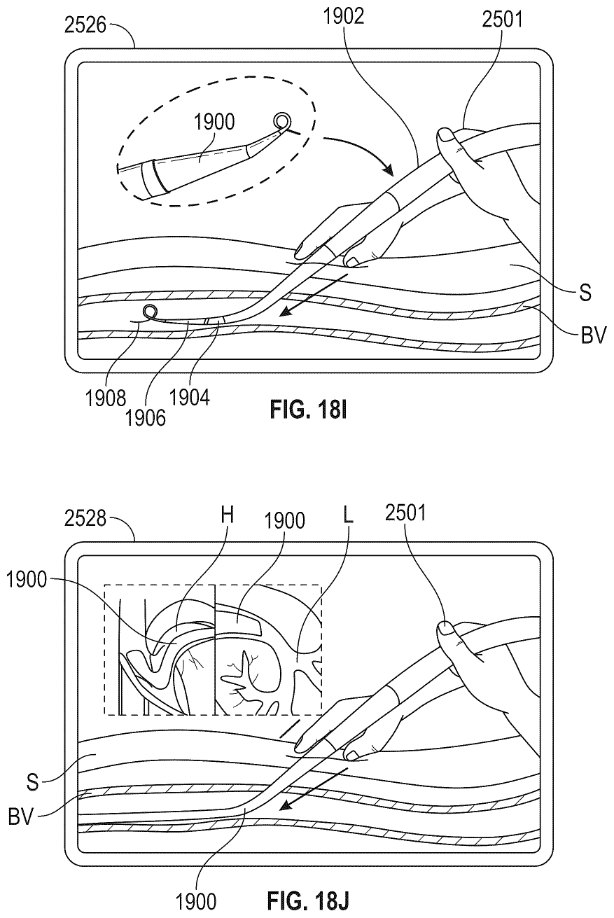

Initially, the multi-device assembly 2900 can be introduced into a patient's blood vessel (e.g., the femoral vein). In some embodiments, the multi-device assembly 2900 can be introduced into the patient's blood vessel via the insertion method depicted in FIGS. 18A-18J. In some embodiments, the guide catheter 2906 and a dilator can be placed into a blood vessel (e.g., femoral vein, jugular vein, etc.) over an access wire. The guide catheter 2906 may act as a sheath. Once the guide catheter 2906 is positioned within the vein, the dilator and access wire may be removed. The procedure catheter 2904, insert catheter 2902, and the guidewire 2907 can be inserted into the guide catheter 2906 to complete the stack within the patient's blood vessel.

After being introduced into the patient's blood vessel (e.g., femoral vein), the insert catheter 2902, the procedure catheter 2904, the guide catheter 2906, and the guidewire 2907 can be advanced (e.g., simultaneously or individually) up the inferior vena cava and into the right atrium of the patient's heart. Next, the insert catheter 2902 and the guidewire 2907 can be rotated and/or advanced (e.g., simultaneously or individually) through the right atrium, into the right ventricle, then into the pulmonary trunk.

Once in the pulmonary trunk, contrast mapping can be performed through the insert catheter 2902 to identify clot burden and establish a target treatment site (e.g., the location of the blood clot). In some embodiments, the contrast mapping can be performed without needing to remove the guidewire 2907 from the insert catheter 2902. For example, the inner diameter of the insert catheter 2902 and the outer diameter of the guidewire 2907 can be sized to enable contrast media to flow between the insert catheter 2902 and the guidewire 2907. In some embodiments, a minimum space of about 0.027 inches between the inner diameter of the insert catheter 2902 and the outer diameter of the guidewire 2907 can be maintained to facilitate a sufficient flow rate of the contrast media.

Next, the insert catheter 2902 and the guidewire 2907 can be rotated and/or advanced (e.g., simultaneously or individually) into the desired branch of the lung and to the target treatment site (e.g., into a lower pulmonary artery of the right lung). Once the insert catheter 2902 and the guidewire 2907 are at the target treatment site, the procedure catheter 2904 can be advanced to the treatment site (e.g., the right pulmonary branch) over the insert catheter 2902 and the guidewire 2907.

Once the procedure catheter 2904, the insert catheter 2902, and the guidewire 2907 are at the treatment site, the guide catheter 2906 can be advanced over the previously advanced components (e.g., the procedure catheter 2904, the insert catheter 2902, and the guidewire 2907) through the pulmonary trunk PT (e.g., into the main pulmonary artery). With the guide catheter 2906 advanced into the main pulmonary artery, the guide catheter 2906 can maintain access (e.g., without the need for exchange wires). In some embodiments, the guide catheter 2906 and the procedure catheter 2904 can be advanced together. In other embodiments, the guide catheter 2906 and the procedure catheter 2904 can be advanced and/or adjusted individually. With the guide catheter 2906 and the procedure catheter 2904 placed in the main pulmonary artery, the insert catheter 2902 and the guidewire 2907 can be removed (e.g., simultaneously or individually), leaving the procedure catheter 2904 in place to initiate thrombectomy (e.g., aspiration). In some embodiments, the guide catheter 2906 may be used for aspiration in addition to or alternatively to the procedure catheter 2904.

If repositioning to another part of the pulmonary anatomy is needed (e.g., for additional thrombectomy), the guide catheter 2906 and the procedure catheter 2904 can be retracted, rotated, and/or advanced (e.g., simultaneously or individually) into desired locations/branches of the pulmonary anatomy with or without reinsertion of the insert catheter 2902 and/or the guidewire 2907. When repositioning, the guide catheter 2906 and the procedure catheter 2904 can either be advanced together or advanced individually. If needed for repositioning, the insert catheter 2902 and the guidewire 2907 can be reinserted back into the guide catheter 2906 and the procedure catheter 2904 to help navigate pulmonary vasculature. Contrast mapping may be performed through any of the available devices, at any time during the procedure.

As described herein, in various steps of a procedure for treating a pulmonary embolism, multiple components of the multi-catheter assembly may be rotated, advanced, and/or adjusted simultaneously or individually. Simultaneous movement may be advantageous for ease of user control, to provide for a faster procedure, and/or to provide a combination of interventional devices having combined desired properties (e.g., stiffnesses) at particular locations within the anatomy.

As described herein, in certain embodiments, an insert or access catheter (such as insert or access catheter 2902) may be used in a procedure for treating pulmonary embolism (e.g., to provide access to anatomical areas for other interventional devices in a stack, for example, by gaining access before advancing the other interventional devices to the anatomical areas). In other embodiments, other access devices, such as an access dilator, may be used. The access devices described herein may provide support to other devices (e.g., catheters) while advancing those devices within the vasculature. The access devices described herein may provide atraumatic distal tips for safe navigation within the vasculature. In certain embodiments, an access dilator may not include an open lumen extending therethrough. In other embodiments, an access dilator may include a lumen that may receive one or more additional devices or fluids therethrough, e.g., in a similar manner to an insert or access catheter. While certain embodiments herein describe examples of particular access devices, such as an insert or access catheter or an access dilator, it is contemplated that other embodiments of access devices described herein may be utilized in the same or similar manner and/or may have any of the same or similar features and/or functions. For example, an access dilator (e.g., access dilator 2800, 3200, 3300) may have any of the same and/or similar features and/or functions as an insert catheter or access catheter (e.g., insert catheter 1906, 2902) and vice versa and/or may be used in any of the same and/or similar procedures. For example, an access dilator (e.g., access dilator 2800, 3200, 3300) may be used in the procedure described with respect to FIGS. 17A-17U or the procedure described with respect to FIGS. 18A-18J. Accordingly, while described with examples of specific insert catheters, the procedures of FIGS. 17A-17U and 18A-18J are not limited to the particular example insert catheters described, but may be performed using other access devices as described herein or may be described as generally being performed by an access device.

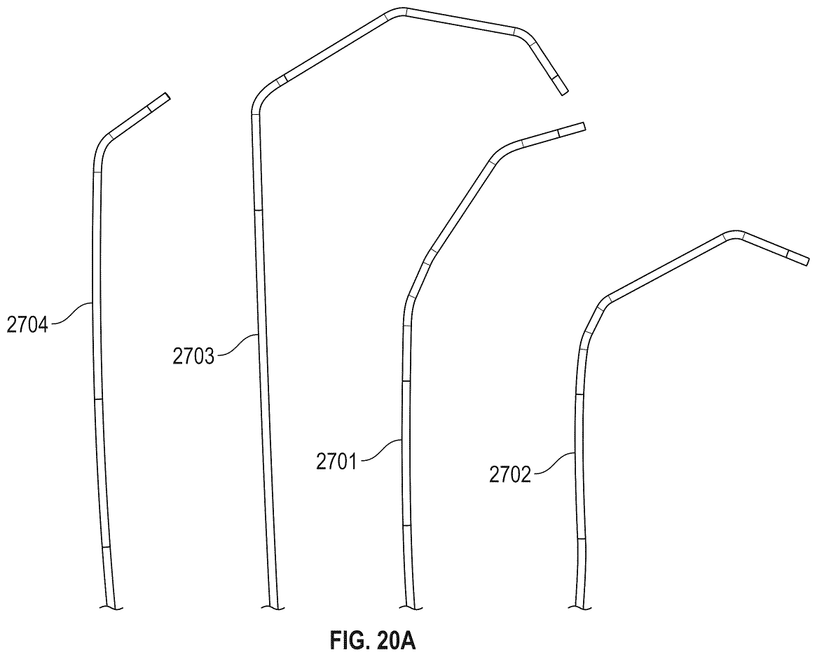

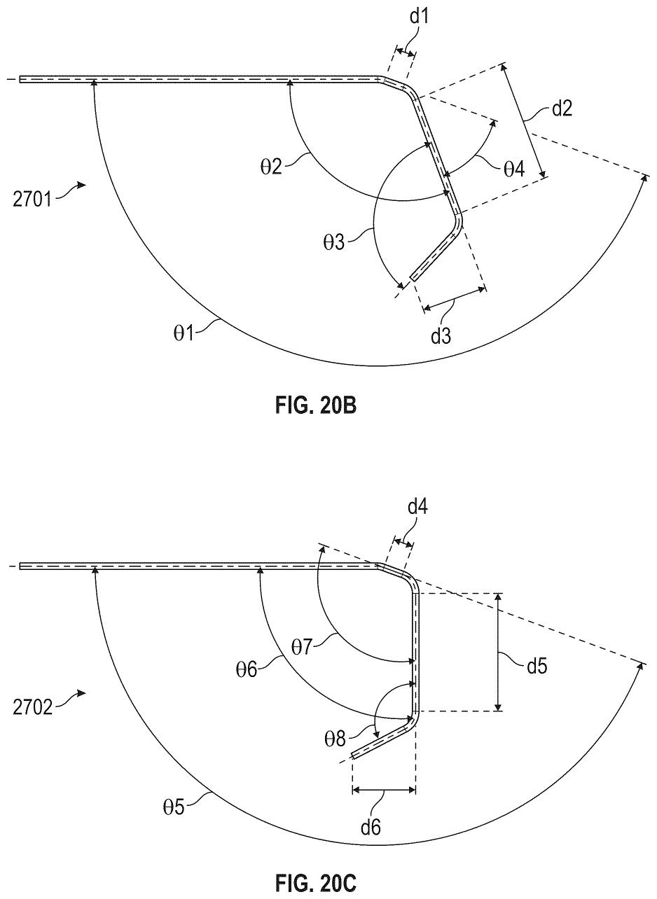

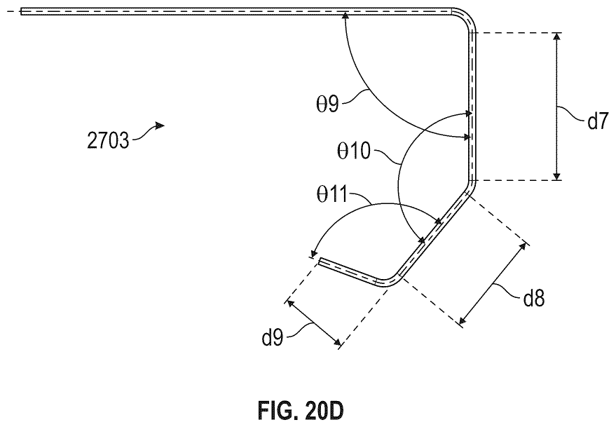

FIG. 20A depicts example insert catheters 2902 that can be used with the multi-device assembly 2900 to treat pulmonary embolism. In various embodiments, the insert catheter 2902 can have any one of a first shape 2701, a second shape 2702, a third shape 2703, or a fourth shape 2704.

FIG. 20B depicts a schematic view of the first shape 2701 for the insert catheter 2902. As shown in FIG. 20B, the first shape 2701 can include distances d1, d2, d3 and angles θ1, θ2, θ3, θ4. According to an example embodiment, the first shape 2701 can include one or more of the following dimensional values: d1=5 mm, d2=30 mm, d3=16 mm, θ1=160 degrees, θ2=110 degrees, θ3=117 degrees, θ4=50 degrees.

FIG. 20C depicts a schematic view of the second shape 2702 for the insert catheter 2902. The second shape 2702 can provide improved throw for accessing the right ventricle. The second shape 2702 may be used with the guidewire 2907 to get access to the pulmonary artery. As shown in FIG. 20C, the second shape 2702 can include distances d4, d5, d6 and angles θ5, θ6, θ7, θ8. According to an example embodiment, the second shape 2702 can include one or more of the following dimensional values: d4=5 mm, d5=30 mm, d6=16 mm, θ5=160 degrees, θ6=90 degrees, θ7=110 degrees, θ8=117 degrees.

FIG. 20D depicts a schematic view of the third shape 2703 for the insert catheter 2902. The third shape 2703 can advantageously provide a better throw from the inferior vena cava to the right ventricle (e.g., through the right atrium). The third shape 2703 can directly hook into the pulmonary artery extending from the inferior vena cava (e.g., through the right atrium and the right ventricle). With a slight push and a half rotation, an insert catheter 2902 with the third shape 2703 can jump into the pulmonary artery. In some embodiments, the third shape 2703 can enable tracking of the insert catheter 2902 straight to the left pulmonary artery. The middle bend of the third shape 2703 can facilitate access to the right pulmonary artery. As shown in FIG. 20D, the third shape 2703 can include distances d7, d8, d9 and angles θ9, θ10, θ11. According to an example embodiment, the third shape 2703 can include one or more of the following dimensional values: d7=35 mm, d8=25 mm, d9=16 mm, θ9=90 degrees, θ10=140 degrees, θ11=110 degrees. FIGS. 20B-20D provide dimensions for the first shape 2701, the second shape 2702, the third shape 2703. In other embodiments, any one or more of the values of dimensions d1, d2, d3, d4, d5, d6, d7, d8, d9, θ1, θ2, θ3, θ4, θ5, θ6, θ7, θ8, θ9, θ10, θ11 provided above and corresponding to FIGS. 20B-20D can be increased or decreased by about 1%, about 5%, about 10%, about 15%, about 25%, about 50%, about 75%, about 100%, any value between about 0% and about 100%, or over about 100%. In certain embodiments, an access dilator (e.g., access dilator 2800, 3200, 3300).

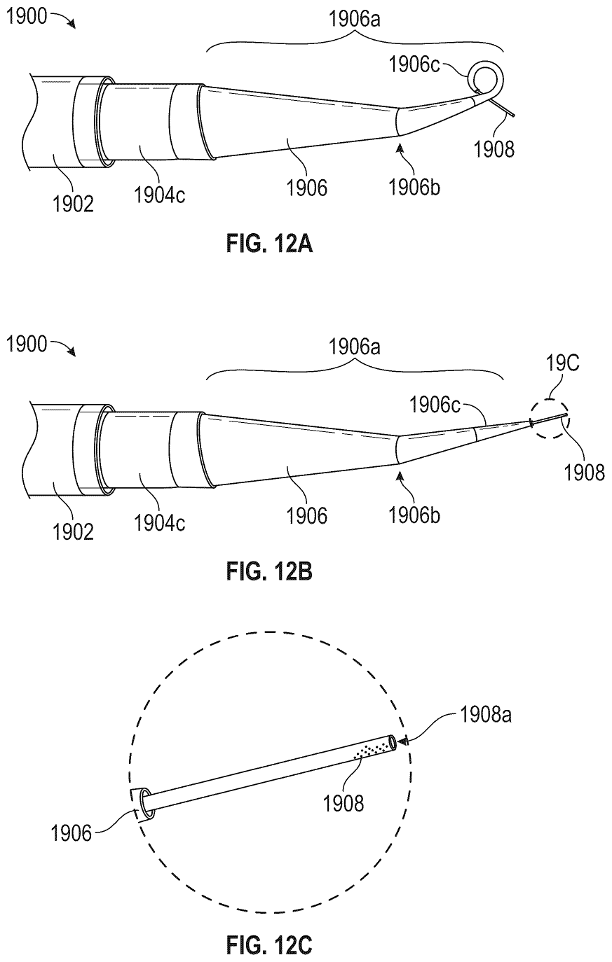

FIG. 12A depicts a schematic view of a multi-device stack 1900 (e.g., a multi-catheter stack 1900) that can be used for treatment of pulmonary embolism. The multi-device stack 1900 can advantageously provide direct access to a treatment site within a patient's lungs. The multi-device stack 1900 can include a sheath 1902, a procedure catheter 1904, an access device, which may be in the form of an insert catheter 1906, and a guidewire 1908. In certain embodiments, the multi-device stack 1900 may include only a subset of these devices. As described in more detail below, the multi-device stack 1900 can be controlled by a robotic drive system (e.g., the drive system 18, the drive system 2818) to advance the multi-device stack 1900 through a patient's vasculature and to a treatment site. Additional details of the robotic drive system are disclosed in U.S. application Ser. No. 18/986,519 filed Dec. 18, 2024, entitled ROBOTIC HUB ASSEMBLY, the entirety of which is hereby expressly incorporated by reference herein. In other embodiments, the multi-device stack 1900 may be manually driven.

As shown in FIG. 12A, the multi-device stack 1900 can be assembled in a concentric stack. The sheath 1902, the procedure catheter 1904, the insert catheter 1906, and the guidewire 1908 can be arranged in a nested configuration within each other. Specifically, the sheath 1902 can be the radially outermost component of the multi-device stack 1900. The procedure catheter 1904 can be concentrically nested within the sheath 1902. The insert catheter 1906 can be concentrically nested within the procedure catheter 1904. The guidewire 1908 can be concentrically nested within the insert catheter 1906. FIG. 12A shows the multi-device stack 1900 arranged such that the guidewire 1908 extends slightly beyond the distal end of the insert catheter 1906, the insert catheter 1906 extends slightly beyond the distal end of the procedure catheter 1904, and the procedure catheter 1904 extends slightly beyond the distal end of the sheath 1902. However, one of skill in the art would understand that these devices may be moved relative to one another to different configurations as described herein. As provided below, each component of the multi-device stack 1900 (e.g., the sheath 1902, the procedure catheter 1904, the insert catheter 1906, and the guidewire 1908) can be defined by a total length and a working length. The total length can be defined as the length between the proximal end of the component and the distal end of the component. The working length can be defined as the maximum length that the component can be advanced into a patient beyond an access point in the patient's body. As described below, the access point can be a puncture into the patient's femoral vein, jugular vein, or other blood vessel. The working length may be less than the total length because at least some portion of the component may need to extend out of the patient's body proximally from the access point to enable manipulation of the component by a medical practitioner or a robotic drive system. For example, a portion of the component extending proximally from the access point can be recessed within a hub of a robotic drive system as described herein. Additionally, there may be an additional distance between a distal most location that a hub may advance to along a drive surface of a robotic drive system and an access point (e.g., due to the architecture of the robotic drive system, due to docking components of the robotic drive system, due to a distance that a separate introducer sheath extends proximally out of the body, etc.). In some embodiments, the total length of any of the components of the multi-device stack 1900 can be within a range of between about 1 cm to about 40 cm greater than the working length, within a range of about 5 cm to about 30 cm greater than the working length, within a range of about 10 cm to about 20 cm greater than the working length, at least about 5 cm greater than the working length, at least about 15 cm greater than the working length, or at least about 30 cm greater than the working length. In this same respect, the working length of any of the components of the multi-device stack 1900 can be within a range of about 1 cm to about 40 cm less than the total length, within a range of about 5 cm to about 30 cm less than the total length, within a range of about 10 cm to about 20 cm less than the total length, at least about 5 cm less than the total length, at least about 15 cm less than the total length, or at least about 30 cm less than the total length.

According to one example corresponding to an access point in a patient's femoral vein, a patient can have a vasculature length from the access point in the femoral vein to the mid-main pulmonary trunk of between about 45 cm to about 55 cm. Accordingly, the working length of any of the components of the multi-device stack 1900 can be within a range of at least about 45 cm to at least about 55 cm. The working length of any of the devices may be greater to enable navigation to a different target site within the patient's vasculature (e.g., to the lower right lobe of the pulmonary arteries). In some embodiments, the working length of any of the components of the multi-device stack 1900 can be at least about 30 cm, at least about 45 cm, at least about 55 cm, at least about 60 cm, at least about 70 cm, between about 30 cm to about 80 cm, between about 55 cm to about 75 cm, between about 60 cm to about 70 cm, or any other value encompassed by the preceding ranges.

One or more of the sheath 1902, the procedure catheter 1904, the insert catheter 1906, and the guidewire 1908 can be coupled to a unique interventional device hub as described herein. In some embodiments, each of the sheath 1902, the procedure catheter 1904, the insert catheter 1906, and the guidewire 1908 can be coupled to a unique interventional device hub as described herein.

One concern associated with existing techniques and devices for treatment of PE (or other diseases) is the risk of losing access to the treatment site midway through a procedure. If access to the treatment site is lost at any point during the procedure (e.g., due to the treatment device being retracted too far), the physician must repeat the time-consuming process of re-advancing the treatment device through the patient's anatomy and to the treatment site. Loss of treatment site access extends the duration of the procedure, thereby increasing the risk of patient injury or death. Existing treatment techniques often require the insertion and placement of long access wires to maintain access to the treatment site. However, such access wires add complexity to the procedure, can pose a risk of injury to the patient (e.g., perforation of the blood vessel) if advanced too far, and can still easily lose access to the treatment site if retracted too far. Additionally, such access guidewires may need to be retracted prior to performing certain procedural steps, such as clot aspiration. Accordingly, if further procedural steps are required after aspiration, an access guidewire may need to be re-advanced through the patient's anatomy.

As described in more detail below, the sheath 1902 can extend from an initial entry site (e.g., access point) in the patient (e.g., a puncture in the patient's skin), through the patient's vasculature, and to the target treatment site (or a position immediately adjacent thereto) (e.g., to the pulmonary trunk). In this respect, the sheath 1902 can advantageously function as a large conduit to provide access (or re-access) to the treatment site for any of the other components of the multi-device stack 1900 or for any other devices advanced within the sheath 1902. The sheath 1902 can remain at or near the treatment site for the duration of the procedure to advantageously provide access (or re-access) to the treatment site at any time during the procedure. Accordingly, the sheath 1902 can advantageously reduce the risk of loss of access to the treatment and replace and/or eliminate the need for access wires to be used in PE or similar treatment procedures.

With reference to FIG. 12A, the sheath 1902 can be a large bore catheter having a size (e.g., inner diameter) of at least about 8 F, at least about 12 F, at least about 16 F, at least about 30 F, at least about 40 F, within a range between about 8 F to about 40 F, or within a range between about 12 F to about 30 F, e.g., depending upon desired clinical performance. In some embodiments, the sheath 1902 can preferably be a 30 F catheter with a 30 F proximal inner diameter and a 30 F distal inner diameter. The larger bore size of the sheath 1902 can advantageously enable larger nested devices (e.g., a larger procedure catheter 1904) to be advanced to the treatment site. Additionally, the larger bore size of the sheath 1902 can advantageously reduce the risk of corking or blockage of the sheath 1902 (e.g., by thrombus T). The sheath 1902 can have any suitable total length, for example a total length of at least about 45 cm, at least about 75 cm, at least about 100 cm, at least about 135 cm, at least about 150 cm, within a range from about 45 cm to about 150 cm, or within a range from about 50 cm to about 135 cm, e.g., depending upon desired clinical performance. In some embodiments, the sheath 1902 can preferably have a total length of about 85 cm. In some embodiments, the sheath 1902 can preferably have a total length of about 100 cm. As referenced above, the working length of the sheath 1902 can be within a range of about 1 cm to about 40 cm less than the total length, within a range of about 5 cm to about 30 cm less than the total length, within a range of about 10 cm to about 20 cm less than the total length, at least about 5 cm less than the total length, at least about 15 cm less than the total length, or at least about 30 cm less than the total length. In some embodiments, the working length of the sheath 1902 can be between about 30 cm and about 135 cm, between about 40 cm and about 100 cm, between about 50 cm and about 90 cm, or between about 60 cm and about 80 cm. In some embodiments, the working length of the sheath can be at least about 30 cm, at least about 40 cm, at least about 50 cm, at least about 60 cm, at least about 70 cm, at least about 80 cm, at least about 90 cm, at least about 100 cm, at least about 110 cm, at least about 120 cm, or more. In some embodiments, the working length of the sheath 1902 can be about 70 cm. The sheath 1902 can be relatively soft and/or flexible to facilitate traversal through the patient's vasculature. In some embodiments, the sheath 1902 can be a guide catheter or function as a guide catheter. The sheath 1902 may include any of the same and/or similar features and functions as any of the guide catheters described herein. In certain embodiments, the sheath 1902 can be fluidly connected to a vacuum source (e.g., a pump) for clot removal.

With reference to FIG. 12A, the procedure catheter 1904 can be an aspiration catheter. The procedure catheter 1904 can function as a thrombectomy catheter to perform aspiration of a blood clot (e.g., a pulmonary embolism). The procedure catheter 1904 can be fluidly connected to a vacuum source (e.g., a pump). The procedure catheter 1904 can include a distal opening to provide aspiration (e.g., vacuum from the pump) for clot removal (e.g., removal of a thrombus and/or embolus). The procedure catheter 1904 can have a size (e.g., outer diameter) of at least about 8 F, at least about 12 F, at least about 16 F, at least about 30 F, at least about 40 F, within a range between about 8 F to about 40 F, or within a range between about 12 F to about 30 F, e.g., depending upon desired clinical performance. In some embodiments, the outer diameter of the procedure catheter 1904 can be chosen to be about the same size as the inner diameter of the sheath 1902. In some embodiments, the procedure catheter 1904 can preferably be a 24 F catheter with a 24 F proximal inner diameter and a 24 F distal inner diameter. In some embodiments, the procedure catheter 1904 can be smaller than 8 F. The procedure catheter 1904 can have any suitable total length, for example a total length of at least about 50 cm, at least about 60 cm, at least about 75 cm, at least about 100 cm, at least about 125 cm, at least about 165 cm, at least about 200 cm, within a range from about 50 cm to about 200 cm, or within a range from about 60 cm to about 165 cm, e.g., depending upon desired clinical performance. In some embodiments, the total length of the procedure catheter 1904 can be about 10 cm to about 30 cm or longer than the total length of the sheath 1902. In some embodiments, the procedure catheter 1904 can preferably have a total length of 85 cm. In some embodiments, the procedure catheter 1904 can preferably have a total length of about 100 cm. As referenced above, the working length of the procedure catheter 1904 can be within a range of about 1 cm to about 40 cm less than the total length, within a range of about 5 cm to about 30 cm less than the total length, within a range of about 10 cm to about 20 cm less than the total length, at least about 5 cm less than the total length, at least about 15 cm less than the total length, or at least about 30 cm less than the total length. In some embodiments, the working length of the procedure catheter 1904 can be between about 30 cm and about 165 cm, between about 40 cm and about 100 cm, between about 50 cm and about 90 cm, or between about 60 cm and about 80 cm. In some embodiments, the working length of the procedure catheter 1904 can be at least about 30 cm, at least about 40 cm, at least about 50 cm, at least about 60 cm, at least about 70 cm, at least about 80 cm, at least about 90 cm, at least about 100 cm, at least about 110 cm, at least about 120 cm, at least about 130 cm, at least about 140 cm or more. In some embodiments, the working length of the procedure catheter 1904 can be about 70 cm. In some embodiments, the working length of the procedure catheter 1904 can be about 85 cm. In some embodiments, the procedure catheter 1904 can include an atraumatic, beveled tip for clot ingestion. In some embodiments, the procedure catheter 1904 can include a support profile that is mapped for delivery through the heart and into the pulmonary arteries. In some embodiments, an initially larger diameter procedure catheter 1904 (e.g., 30 F) can be exchanged for a smaller diameter procedure catheter 1904 (e.g., 16 F) to enable the procedure catheter 1904 to reach smaller diameter or more distally positioned treatment sites. In some embodiments, the procedure catheter 1904 can include a telescoping distal end with a smaller diameter (e.g., 16 F) for distal vessel access. In some embodiments, the procedure catheter 1904 may include any of the same and/or similar features and functions as any of the procedure catheters described herein.

With reference to FIG. 12A, the insert catheter 1906 can function as a leading device to guide the multi-device stack 1900 through the patient's vasculature and to the target treatment site. Specifically, in the context of PE treatments, the insert catheter 1906 can guide and facilitate traversal of the multi-device stack 1900 through the tortuous anatomy of the heart H. The insert catheter 1906 can have a size (e.g., a maximum outer diameter) of at least about 8 F, at least about 12 F, at least about 16 F, at least about 30 F, at least about 40 F, within a range between about 8 F to about 40 F, or within a range between about 12 F to about 30 F, e.g., depending upon desired clinical performance. As shown in FIG. 12A, the insert catheter 1906 can include a tapered distal end portion 1906a. The tapered distal end portion 1906a can include at least the portion of the insert catheter 1906 that extends beyond the distal end of the procedure catheter 1904. The maximum outer diameter of the insert catheter 1906 can be at the position at which the insert catheter 1906 extends out of the procedure catheter 1904. The insert catheter 1906 can have a minimum outer diameter at its distal tip. In this manner, the tapered distal end portion 1906a can provide a smooth (e.g., seamless) and atraumatic transition between the procedure catheter 1904 and the insert catheter 1906, thereby minimizing the risk of vessel trauma (e.g., spasm or arrhythmic reaction of the heart). In some embodiments, the minimum or distal outer diameter of the insert catheter 1906 can be about 50% of its maximum outer diameter, less than about 50% of its maximum outer diameter, or less than about 25% of its maximum outer diameter. For example, in some embodiments, the insert catheter 1906 can preferably have a proximal outer diameter of about 24 F, a proximal inner diameter of less than about 24 F, a distal outer diameter of about 12 F, a distal inner diameter of about 8 F, and a wall thickness of about 4 F. The tapered distal end portion 1906a can facilitate traversal within the patient's vasculature. The insert catheter 1906 can have any suitable total length, for example a total length of at least about 45 cm, at least about 55 cm, at least about 75 cm, at least about 100 cm, at least about 125 cm, at least about 150 cm, at least about 185 cm, at least about 200 cm, at least about 250 cm, within a range from about 45 cm to about 250 cm, or within a range from about 55 cm to about 185 cm, e.g., depending upon desired clinical performance. In some embodiments, the insert catheter 1906 can preferably have a total length of about 100 cm. In some embodiments, the insert catheter 1906 can preferably have a total length of about 115 cm. As referenced above, the working length of the insert catheter 1906 can be within a range of about 1 cm to about 40 cm less than the total length, within a range of about 5 cm to about 30 cm less than the total length, within a range of about 10 cm to about 20 cm less than the total length, at least about 5 cm less than the total length, at least about 15 cm less than the total length, or at least about 30 cm less than the total length. In some embodiments, the working length of the insert catheter can be between about 35 cm and about 185 cm, between about 50 cm and about 130 cm, between about 60 cm and about 120 cm, between about 70 cm and about 100 cm. In some embodiments, the working length of the insert catheter 1906 can be at least about 35 cm, at least about 40 cm, at least about 50 cm, at least about 60 cm, at least about 70 cm, at least about 80 cm, at least about 90 cm, at least about 100 cm, at least about 110 cm, at least about 120 cm, at least about 130 cm, at least about 140 cm, at least about 150 cm, at least about 160 cm or more. In some embodiments, the working length of the insert catheter 1906 can be about 85 cm. In some embodiments, the working length of the insert catheter 1906 can be about 100 cm. In some embodiments, the insert catheter 1906 can include anatomy-specific support zones for delivery into the pulmonary arteries. In some embodiments, a central lumen 1908a of the insert catheter 1906 can be shaped and/or sized to allow for contrast injection without removal of the guidewire 1908. As shown in FIG. 12A, the tapered distal end portion 1906a of the insert catheter 1906 can additionally include an angled bend 1906b. The angled bend 1906b can facilitate advancement of the insert catheter 1906 through circuitous and/or rounded anatomy.

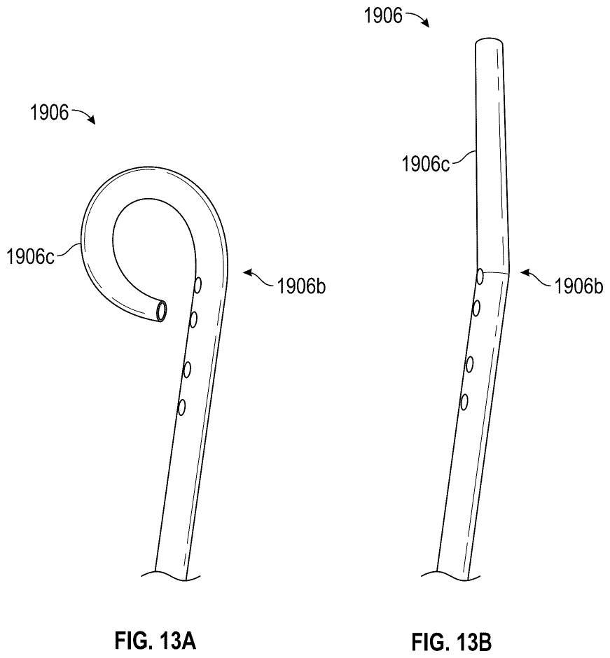

With continued reference to FIG. 12A, in some embodiments, the insert catheter 1906 can include a tip 1906c. The tip 1906c can form a portion of and/or extend from the tapered distal end portion 1906a. FIGS. 13A-13B depict enlarged views of the tip 1906c. The tip 1906c can be positioned distal to the angled bend 1906b. In some embodiments, the portions of the tapered distal end portion 1906a proximal to the tip 1906c may be referred to as an obturator portion., and the tip 1906c may be referred to as a tip portion.

In certain embodiments, the tip 1906c can be a curved tip (e.g., a pigtail tip) or can be manipulated to be shaped as a curved tip (e.g., a pigtail tip) during a procedure. A pigtail tip can reduce a risk of the multi-device stack 1900 being caught or trapped in chordae of the heart H. Further, the tip 1906c can advantageously enable atraumatic traversal through the heart. Specifically, the rounded shape of the pigtail can reduce a risk of injury. Further, the tip 1906c may be used as a leading device and through the heart and can reduce a risk of injury (e.g., perforation of heart walls) that may otherwise occur if a wire or other device was used as the leading device through the heart.

As shown in FIGS. 12A-12B and 13A-13B, the tip 1906c can be moved between a curled or curved configuration (e.g., a pigtail configuration) and a straightened configuration. In this respect, the insert catheter 1906 can be shape-shifted from a pigtail-shaped tip to a straightened tip. The insert catheter 1906 may also be torque-able. FIGS. 12A and 13A show the tip 1906c in the curved configuration. When the tip 1906c is in the curved configuration, the tip 1906c can be curled up to form a rounded distal tip surface.

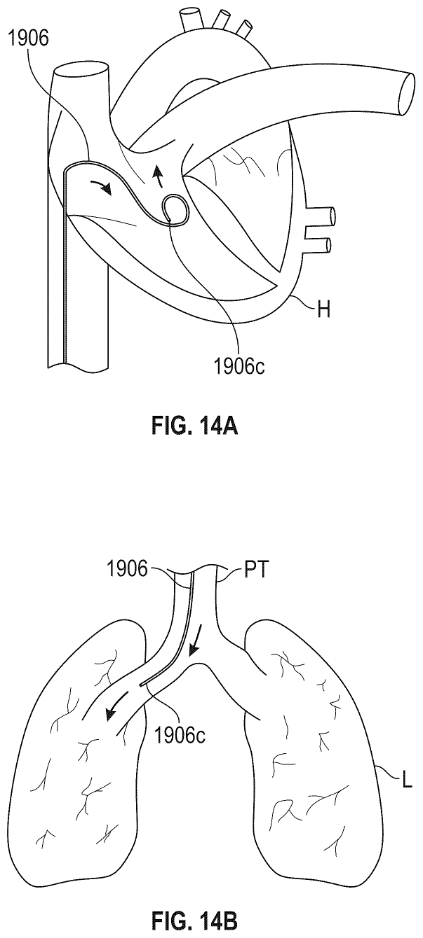

FIG. 14A depicts a schematic view of the insert catheter 1906 being traversed through the heart H. As shown in FIG. 14A, the tip 1906c can preferably be maintained in the curled configuration at least during traversal of the multi-device stack 1900 through the heart H to facilitate atraumatic advancement. FIGS. 12B and 13B show the tip 1906c in the straightened configuration. When the tip 1906c is in the straightened configuration, the tip 1906c can be at least partially straightened. FIG. 14B depicts a schematic view of the insert catheter 1906 being traversed through the lungs L. As shown in FIG. 14B, the tip 1906c can preferably be maintained in the straightened configuration at least when the multi-device stack 1900 is positioned at the treatment site (e.g., within the pulmonary trunk PT of the lungs L) to facilitate accurate advancement of the guidewire 1908 into more distal regions of the anatomy (e.g. into the pulmonary arteries). In some embodiments, the tip 1906c can be self-straightening (e.g., via a straightening mechanism integrated into the insert catheter 1906). In some embodiments, the tip 1906c can be straightened by advancement of the guidewire 1908 through and out of the tip 1906c (or additional devices such as other guidewires or catheters). In other embodiments, the tip 1906c may not be able to transition between the curled configuration and the straightened configuration. Rather, the tip 1906c may be permanently maintained in the curved configuration.

While FIGS. 12A-12B and 13A-13B depict one example insert catheter 1906 with a tapered distal end portion 1906a, an angled bend 1906b, and a tip 1906c, it is to be understood that other embodiments of the insert catheter 1906 can be used with the multi-device stack 1900. In some embodiments, the insert catheter 1906 can include any one of an obtuse angled pigtail catheter (e.g., Nyman-type), a substantially right-angled pigtail catheter (e.g., Grollman-type), a straight pigtail catheter (e.g., Eppendorf-type), a balloon occlusion catheter (e.g., Berman-type), or a double-angled catheter (e.g., a “hockey stick” catheter with a 60-degree bend). The insert catheter 1906 may have any of the same features and/or functions as any of the other insert catheters described herein.

FIG. 12C depicts an enlarged view of a region of the multi-device stack 1900 showing the guidewire 1908. As shown in FIG. 12C, in certain embodiments, the guidewire 1908 can be a hollow wire or microcatheter having a central lumen 1908a. Similar to the insert catheter 1906, in certain embodiments, the guidewire 1908 can be moved between a curled or curved configuration and a straightened configuration.

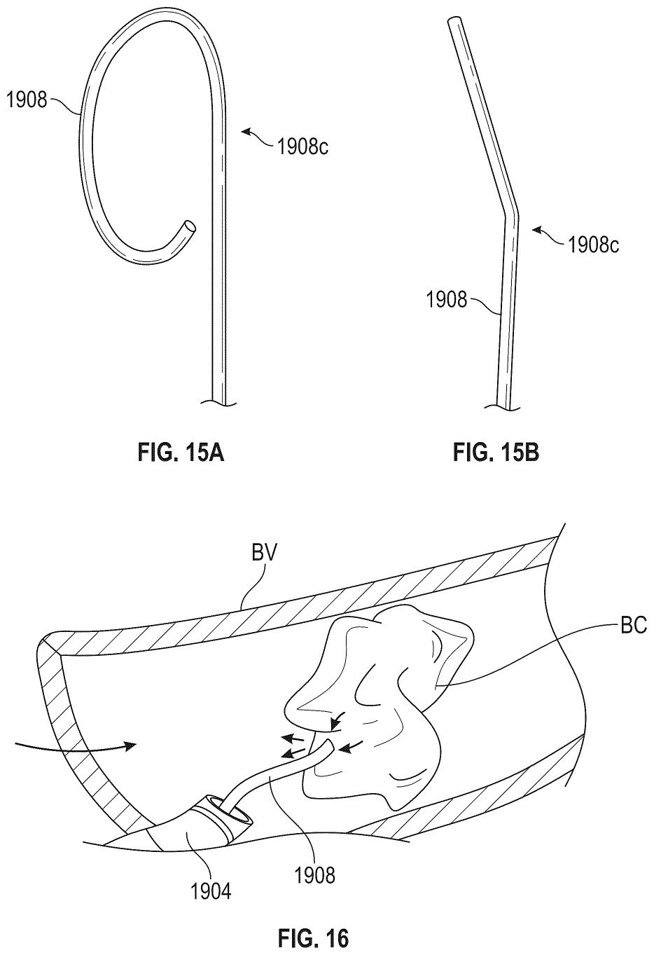

FIG. 15A depicts an enlarged schematic view of the guidewire 1908 in the curled or curved configuration, and FIG. 15B depicts an enlarged schematic view of the guidewire 1908 in the straightened configuration. In some embodiments, the guidewire 1908 can conform to the shape of the insert catheter 1906. For example, the guidewire 1908 can be placed in the curved configuration or the straightened configuration as a result of the insert catheter 1906 being placed in the curved configuration or the straightened configuration, respectively. In some embodiments, the guidewire 1908 can transition between the curved and straightened configurations to cause the insert catheter 1906 to transition between the curved and straightened configurations. In other embodiments, the guidewire 1908 can be independently transitioned between the curved configuration and the straightened configuration. In some embodiments, the guidewire 1908 can have an angled bend 1908c. The angled bend 1908c can arise from the guidewire 1908 conforming to the angled bend 1906b in the insert catheter 1906. In other embodiments, the angled bend 1908c can be formed in the guidewire 1908. The guidewire 1908 can have a size (e.g., outer diameter) of at least about 0.100 inches, at least about 0.070 inches, at least about 0.01 inches, at least about 0.005 inches, within a range from about 0.005 inches to about 0.100 inches or within a range from about 0.010 inches to about 0.070 inches, e.g., depending upon desired clinical performance. In some embodiments, the guidewire 1908 can preferably have a proximal outer diameter of about 6 F, a proximal inner diameter of about 4.2 F, a distal outer diameter of about 6 F, a distal inner diameter of about 4.2 F, and a wall thickness of about 1.8 F. In some embodiments, the guidewire can have an outer diameter of between about 2 F to about 10 F, between about 4 F to about 8 F, or between about 6 F to about 7 F. In some embodiments, the guidewire can have an inner diameter of between about 1.2 F to about 7.2 F, between about 2.2 F to about 6.2 F, or between about 3.2 F to about 5.2 F. In some embodiments, the guidewire can have a wall thickness of between about 0.8 F to about 2.8 F, between about 1.2 F to about 2.4 F, or between about 1.6 F to about 2 F. The guidewire 1908 can have any suitable total length, for example a total length of at least about 25 cm, at least about 40 cm, at least about 75 cm, at least about 100 cm, at least about 150 cm, at least about 200 cm, at least about 250 cm, within a range from about 25 cm to about 250 cm, or within a range from about 40 cm to about 200 cm, e.g., depending upon desired clinical performance. In some embodiments, the guidewire 1908 can preferably have a total length of about 150 cm. In some embodiments, the guidewire 1908 can preferably have a total length of about 165 cm. As referenced above, the working length of the guidewire 1908 can be within a range of about 1 cm to about 40 cm less than the total length, within a range of about 5 cm to about 30 cm less than the total length, within a range of about 10 cm to about 20 cm less than the total length, at least about 5 cm less than the total length, at least about 15 cm less than the total length, or at least about 30 cm less than the total length. In some embodiments, the working length of the guidewire can be between about 20 cm and about 200 cm, between about 80 cm and about 180 cm, between about 100 cm and about 170 cm, or between about 120 cm and about 165 cm. In some embodiments, the working length of the guidewire 1908 can be at least about 20 cm, at least about 40 cm, at least about 50 cm, at least about 60 cm, at least about 70 cm, at least about 80 cm, at least about 90 cm, at least about 100 cm, at least about 110 cm, at least about 120 cm, at least about 130 cm, at least about 140 cm, at least about 150 cm, at least about 160 cm, at least about 170 cm, at least about 180 cm, at least about 190 cm or more. In some embodiments, the working length of the guidewire 1908 can be about 135 cm. In some embodiments, the working length of the guidewire 1908 can be about 150 cm. The guidewire 1908 can be directed into distal patient anatomy and/or treatment sites.

In some embodiments, the guidewire 1908 can perform a plurality of functions. In one aspect, the guidewire 1908 can be used as an access tool to steer to a target location within a patient's vasculature (e.g., for atraumatic vessel selection within a patient's lungs). In another aspect, the central lumen 1908a of the guidewire 1908 can be used as a delivery device to deliver substances, aspiration, and/or other devices to a target location. For example, the guidewire 1908 can be used to deliver drugs (e.g., tissue plasminogen activator (tPA) for clot lysis), liquid embolic agents, contrast media, and/or smaller devices (e.g., a coil to treat perforations). In yet another aspect, the microwire can be used as an anchoring device to lock onto a blood clot or region of anatomy.

FIG. 16 depicts a schematic view of the multi-device stack 1900 at a treatment site within a blood vessel BV. As shown in FIG. 16, the guidewire 1908 can anchor onto a blood clot BC (e.g., via aspiration through the guidewire 1908 and/or via physical engagement with the blood clot). After being anchored to the blood clot, the guidewire 1908 can be used as a rail over which other components of the multi-device stack 1900 (e.g., the procedure catheter 1904) can be advanced closer to the blood clot. Alternatively or additionally, after being anchored to the blood clot, the guidewire 1908 can be used as a “grappling-hook” to pull the blood clot closer to the other components of the multi-device stack 1900 (e.g., the procedure catheter 1904). The guidewire 1908 may have any of the same and/or similar features and functions as any of the other guidewires described herein.

### Example Method of Operating the Multi-Device Stack 1900

A pulmonary embolism can begin as a thrombus in the patient's femoral vein. An embolus can break off from the thrombus and travel through the patient's vasculature until it becomes lodged in the patient's pulmonary arteries, thereby creating a pulmonary embolism. In such a situation, a medical intervention utilizing the multi-device stack 1900 can be undertaken to treat the pulmonary embolism. As mentioned above, the multi-device stack 1900 can advantageously extend from an entry point in the patient's skin to a treatment site in the patient's pulmonary trunk or pulmonary arteries. The multi-device stack 1900 can accordingly provide direct access to the treatment site. In some embodiments, the multi-device stack 1900 can initially be introduced into the patient's vasculature at an entry point into the patient's femoral vein. From the entry point in the femoral vein, the multi-device stack 1900 can be advanced into the inferior vena cava. After climbing up the inferior vena cava, the multi-device stack 1900 can be advanced into the right atrium of the heart. From the right atrium, the multi-device stack 1900 can be advanced through the tricuspid valve and into the right ventricle. From the right ventricle, the multi-device stack 1900 can be advanced through the pulmonary valve and into the pulmonary trunk. From the pulmonary trunk, one or more components of the multi-device stack 1900 can be advanced into either the right lung or the left lung, depending on the location of the pulmonary embolism. Within either the right lung or the left lung, the multi-device stack 1900 can be advanced into a pulmonary artery and to a treatment site within the pulmonary artery. The treatment site can be at and/or adjacent to the location of the pulmonary embolism. In some embodiments, the multi-device stack 1900 can alternatively be introduced into the patient's vascular system through an entry point into the jugular vein. From the entry point in the jugular vein, the multi-device stack 1900 can be advanced down into the right atrium. From the right atrium, the multi-device stack 1900 can be advanced to the pulmonary artery in the same manner as described above.

In certain embodiments, initially, the multi-device stack 1900 can be introduced into the patient's blood vessel (e.g., the femoral vein, jugular vein, etc.). In some embodiments, the multi-device stack 1900 can be introduced into the patient's blood vessel via the insertion method depicted in FIGS. 18A-18J. In some embodiments, the sheath 1902 and a dilator can be placed into a blood vessel (e.g., femoral vein, jugular vein, etc.) over an access wire. Once the sheath 1902 is positioned within the vein, the dilator and access wire may be removed. The procedure catheter 1904, insert catheter 1906, and guidewire 1908 can be inserted into the sheath 1902 to complete the stack within the patient's blood vessel. In some cases, the multi-device stack 1900 can be inserted through an additional larger sheath. For example, in some embodiments, the multi-device stack 1900 can be inserted through an introduced sheath, such as introducer sheath 3002. In some embodiments, the stack 1900 can be introduced through a short sheath (e.g., shorter than the sheath 1902). In some embodiments, the additional sheath can be manually placed.

After being introduced into the patient's blood vessel (e.g., femoral vein), the sheath 1902, the procedure catheter 1904, the insert catheter 1906, and the guidewire 1908 can be advanced (e.g., simultaneously or individually) up the inferior vena cava and into the right atrium of the patient's heart. Next, the insert catheter 1906 and the guidewire 1908 can be rotated and/or advanced (e.g., simultaneously or individually) through the right atrium, into the right ventricle, then into the pulmonary trunk.

Once in the pulmonary trunk, the sheath 1902 can be advanced over the procedure catheter 1904, the insert catheter 1906, and the guidewire 1908 into a stable (e.g., midpoint) position within the pulmonary trunk (e.g., to complete pulmonary access).

After the sheath 1902 is at a stable position within the pulmonary trunk, contrast mapping can be performed through insert catheter 1906 to identify clot burden and establish a target treatment site (e.g., the location of the blood clot). In some embodiments, the contrast mapping can be performed without needing to remove the guidewire 1908 from the insert catheter 1906. For example, the inner diameter of the insert catheter 1906 and the outer diameter of the guidewire 1908 can be sized to enable contrast media to flow between the insert catheter 1906 and the guidewire 1908. In some embodiments, a minimum space of at least about 0.015 inches, at least about 0.025 inches, at least about 0.027 inches, at least about 0.035 inches, at least about 0.045 inches, at least about 0.065 inches, within a range from about 0.015 inches to about 0.065 inches, or within a range from about 0.027 inches to about 0.045 inches between the inner diameter of the insert catheter 1906 and the outer diameter of the guidewire 1908 can be maintained to facilitate a sufficient flow rate of the contrast media.

Next, the insert catheter 1906 and the guidewire 1908 can be rotated and/or advanced (e.g., simultaneously or individually) into the desired branch of the lung and to the target treatment site (e.g., into a lower pulmonary artery of the right lung). Once the insert catheter 1906 and the guidewire 1908 are at the target treatment site, the procedure catheter 1904 can be advanced to the treatment site (e.g., the right pulmonary branch) over the insert catheter 1906 and the guidewire 1908.

Once the procedure catheter 1904, the insert catheter 1906, and the guidewire 1908 are at the treatment site, the sheath 1902 can be advanced over the previously advanced components (e.g., the procedure catheter 1904, the insert catheter 1906, and the guidewire 1908) through the pulmonary trunk PT (e.g., into the main pulmonary artery). With the sheath 1902 advanced into the main pulmonary artery, the sheath 1902 can maintain access (e.g., without the need for exchange wires). In some embodiments, the sheath 1902 and the procedure catheter 1904 can be advanced together. In other embodiments, the sheath 1902 and the procedure catheter 1904 can be advanced and/or adjusted individually.

With the sheath 1902 and the procedure catheter 1904 placed in the main pulmonary artery, the insert catheter 1906 and the guidewire 1908 can be removed (e.g., simultaneously or individually), leaving the procedure catheter 1904 in place to initiate thrombectomy (e.g., aspiration). In some embodiments, the sheath 1902 may be used for aspiration in addition to or alternatively to the procedure catheter 1904.

If repositioning to another part of the pulmonary anatomy is needed (e.g., for additional thrombectomy), the sheath 1902 and the procedure catheter 1904 can be retracted, rotated, and/or advanced (e.g., simultaneously or individually) into desired locations/branches of the pulmonary anatomy with or without reinsertion of the insert catheter 1906 and/or the guidewire 1908. When repositioning, the sheath 1902 and the procedure catheter 1904 can either be advanced together or advanced individually. If needed for repositioning, the insert catheter 1906 and the guidewire 1908 can be reinserted (e.g., simultaneously or individually) back into the sheath 1902 and the procedure catheter 1904 to help navigate the pulmonary vasculature.

In some embodiments, smaller and/or longer procedure catheters (e.g., thrombectomy catheters) may be telescoped through the procedure catheter 1904 for additional reach into smaller and/or more distal branches. These catheters may be traced over their own tapered-to-fit insert catheter or advanced over the guidewire 1908 or on their own without inner devices. The smaller and/or longer procedure catheters may be rotated to help in navigating/selecting anatomy and/or focusing on a specific target location.

Contrast mapping may be performed through any of the available devices, at any time during the procedure.

As described herein, in various steps of a procedure for treating a pulmonary embolism, multiple components of the multi-catheter assembly may be rotated, advanced, and/or adjusted simultaneously or individually. Simultaneous movement may be advantageous for ease of user control, to provide for a faster procedure, and/or to provide a combination of interventional devices having combined desired properties (e.g., stiffnesses) at particular locations within the anatomy.

FIGS. 17A-17U depict steps (and sub-steps) in an example method of operating the multi-device stack 1900 for treatment of a blood clot (e.g., a pulmonary embolism). During a medical procedure utilizing the multi-device stack 1900, the relative positions and/or configurations of the components of the multi-device stack 1900 (e.g., the sheath 1902, the procedure catheter 1904, the insert catheter 1906, and the guidewire 1908) can be changed depending on the stage of the procedure and/or the anatomy being advanced through (e.g., the position of the distal end of the multi-device stack 1900 relative to the patient's anatomy). Additionally, the relative axial positions of the one or more components of the multi-device stack 1900 can be dynamically changed to alter the cumulative profile and stiffness of the stack. For example, the multi-device stack 1900 can be made progressively softer or stiffer depending on the number of concentric devices at any specific axial position. The stiffness of the multi-device stack 1900 can be altered to facilitate advancement through specific anatomy of the patient. Further, one or more of the devices of the multi-device stack 1900 may be rotated during the example method.

With reference to FIG. 17A, at step 2400, the multi-device stack 1900 can initially be introduced into the patient's vasculature at an entry point. Additional details of an example introduction method for the multi-device stack 1900 are described below and illustrated in FIGS. 18A-18J. Depending on the preference of the patient, the preference of the physician, and/or other medical considerations, the multi-device stack 1900 can be introduced into the patient's vasculature at various entry points on the patient's body. As referenced above, in some embodiments, the entry point can be a puncture through the skin and into the femoral artery (e.g., near the patient's groin). Another possible entry point is a puncture through the patient's skin into the jugular vein (e.g., at the patient's neck). Any other entry point can similarly be utilized so long as it provides access to the treatment site (e.g., the pulmonary arteries). At the entry point, at least the sheath 1902 can be introduced into the patient's vasculature (e.g., into the femoral vein or jugular vein). In some embodiments, the multi-device stack 1900 can be inserted into the patient's vasculature via the method depicted in FIGS. 18A-18J and described in more detail below.

With reference to FIG. 17B, at step 2402, one or more components of the multi-device stack 1900 (e.g., the insert catheter 1906 and the guidewire 1908) can be advanced from the entry point to the pulmonary trunk (e.g., according to the travel path described above).

The advancement of the multi-device stack 1900 can be performed according to one or more sub-steps. For example, FIGS. 17C-17F depict schematic views of the heart H showing stages of advancement of the multi-device stack 1900. As shown in FIG. 17C, with the sheath 1902 in the patient's femoral vein or jugular vein at the entry point, the internal stack (e.g., the insert catheter 1906 and/or the guidewire 1908) can be advanced (e.g., driven) from near the entry point to the base of the heart H (e.g. within the inferior vena cava IVC). Next, as shown in FIG. 17D, the external stack (e.g., the access catheter and the procedure catheter 1904) can be advanced to the base of the heart H. Next, as shown in FIG. 17E, the insert catheter 1906 and the guidewire 1908 can be advanced through the heart H. As mentioned above, the tip 1906c of the insert catheter 1906 can be used to atraumatically navigate the multi-device stack 1900 through the heart H (e.g., from the entry point into the right atrium, through the tricuspid valve, and through the right ventricle up to the pulmonary trunk PT). Specifically, the tip 1906c of the insert catheter 1906 can be maintained in the curled configuration when leading the multi-device stack 1900 through the tortuous anatomy of the heart H. Next, as shown in FIG. 17F, the tip 1906c of the insert catheter 1906 can be moved to the straightened configuration by driving the guidewire 1908 out of the insert catheter 1906 and into the pulmonary trunk PT. In other embodiments, additional devices may be advanced through the tip 1906c to straighten the tip from the curved configuration to the straightened configuration or alternative methods may be utilized to straighten the tip 1906c. The tip 1906c of the insert catheter 1906 can preferably be moved to the straightened configuration to facilitate advancement of the multi-device stack 1900 through the pulmonary trunk PT and into a specific pulmonary artery PA. The narrower profile of the straightened configuration of the tip 1906c can enable the insert catheter 1906 to more easily be advanced into the narrower and more distally located pulmonary arteries PA. As shown in FIG. 17G, which shows a schematic view of the patient's lung L, the insert catheter 1906 and the guidewire 1908 can be advanced to the face of the blood clot within the pulmonary trunk PT or a pulmonary artery PA.

With reference to FIG. 17H, at step 2404, with the insert catheter 1906 in the pulmonary trunk PT, contrast media 2450 can be injected through the lumen of the insert catheter 1906. FIG. 17I depicts a schematic view of contrast media 2450 being injected into the lungs L of the patient via the insert catheter 1906. In some embodiments, the contrast media 2450 can be injected without removal or retraction of the guidewire 1908 from the insert catheter 1906. Following injection of the contrast media 2450, contrast mapping can enable the physician to locate the location of and/or burden of the blood clot BC within the pulmonary arteries PA. The physician can optionally establish a baseline pressure reading via the multi-device stack 1900. Reperfusion and/or atrial pressure readings can be measured.

With reference to FIG. 17J, at step 2406, the guidewire 1908 can be advanced to the blood clot. The guidewire 1908 can anchor onto (e.g., attach to) the blood clot (e.g., via aspiration through the central lumen 1908a of the guidewire 1908 and/or via physical engagement with the blood clot). FIG. 17K depicts a schematic view of the patient's lung L showing the guidewire 1908 anchored to the blood clot. After the guidewire 1908 is anchored to the blood clot, a clot lysing drug (e.g., tPA) can optionally be delivered to the blood clot via the central lumen 1908a of the guidewire 1908 to loosen the blood clot. In other embodiments, the clot lysing drug may be delivered prior to anchoring the guidewire 1908 to the blood clot.

With the guidewire 1908 acting as an anchor and a rail, the sheath 1902 and the procedure catheter 1904 can be advanced over the insert catheter 1906 and the guidewire 1908 to their respective target locations. Specifically, the distal end of the sheath 1902 can be advanced to a stable position within the base of the pulmonary trunk PT, and the distal end of the procedure catheter 1904 can be advanced to a face of the blood clot (e.g., immediately adjacent to or contacting the blood clot) within the pulmonary trunk PT or a pulmonary artery PA. FIG. 17L depicts a schematic view of the patient's heart H after the sheath 1902 has been advanced through the heart H and to its target location at the base of the pulmonary trunk PT. FIG. 17M depicts a schematic view of the patient's lung L after the procedure catheter 1904 has been advanced through the patient's heart H and to its target location at the face of the blood clot within the pulmonary trunk PT. In some embodiments, the guidewire 1908 can be retracted to pull the clot to the distal opening of the procedure catheter 1904. In other embodiments, the guidewire 1908 can be retracted to the face of the procedure catheter 1904 before advancing the sheath 1902 and/or procedure catheter 1904.

With reference to FIG. 17N, at step 2408, once the distal ends of the sheath 1902 and the procedure catheter 1904 are positioned at their respective target locations, the insert catheter 1906 and the guidewire 1908 can be retracted proximally from the distal end of the sheath 1902 and/or removed from the multi-device stack 1900.

With reference to FIG. 17O, at step 2410, with the procedure catheter 1904 at its target location at the blood clot, an aspiration sequence can be performed to aspirate the blood clot. The aspiration sequence can be automated and can include one or more steps of applying aspiration, repositioning the distal end of the procedure catheter 1904, and/or pressure monitoring. In some embodiments, aspiration may be performed through the sheath 1902 (e.g., if the clot is corked to the tip of the procedure catheter 1904).

With reference to FIG. 17P, at step 2412, the aspiration sequence can be stopped (e.g., aspiration turned off). The insert catheter 1906 and the guidewire 1908 can be readied for contrast injection and/or advancement up to the treatment site if needed.

With reference to FIG. 17Q, at step 2414a, the physician and/or other technician can coordinate the aspiration (e.g., thrombectomy) sequencing. For example, the aspiration sequence can be stopped in response to an aspirated blood clot being identified in a clot collection chamber (e.g., a clot catcher) coupled to the multi-device stack 1900. With reference to FIG. 17R, at step 2414b, the clot container can be cleared of the captured blood clot and re-coupled to the multi-device stack 1900.

With reference to FIG. 17S, at step 2416, additional contrast media 2450 can be injected into the patient's lungs L (e.g., at the treatment site). Contrast mapping can be performed to determine the effectiveness of the treatment (e.g., to determine reperfusion, the location of the blood clot, and/or the size of the blood clot). In some instances, after aspiration has been performed the location of the blood clot can change. FIG. 17T depicts a schematic view of the patient's lung L after aspiration showing a change in the location of the blood clot. In some instances, the initial procedure catheter 1904 can be exchanged for a smaller procedure catheter 1904 for repeated aspiration sequences. Steps 2406-2416 can be repeated until the pulmonary embolism has been successfully treated (e.g., until the blood clot has been completely removed and/or until a sufficient mean pressure drop, sufficient reperfusion, or other biometric target value has been reached). During the procedure, the sheath 1902 can provide continual access for retraction and/or advancement of the procedure catheter 1904, the insert catheter 1906, the guidewire 1908, and/or other devices through the sheath 1902.

With reference to FIG. 17U, at step 2418, the entire multi-device stack 1900 can be retracted and removed from the patient. At the conclusion of the procedure, the blood vessel BV at the entry point can be closed.

As described in more detail below, any one or more of steps 2400-2418 (or sub-steps) can be robotically controlled (e.g., by the drive system 18, or drive system 2618). For example, movement (e.g., advancement, retraction, change in configuration) of the sheath 1902, the procedure catheter 1904, the insert catheter 1906, and/or the guidewire 1908 can be robotically controlled (e.g., driven). Furthermore, aspiration, substance injection (e.g., contrast media or drug injection), pressure reading, and other relevant functions can be robotically controlled.

### Example Method of Inserting the Multi-Device Stack

FIGS. 18A-18J depict an example insertion kit 2500 and method of use thereof that can be utilized to introduce an intravascular device (e.g., the multi-device stack 1900) into the vasculature of a patient. The insertion kit 2500 can include a puncture needle 2502, a microwire 2504, and/or a dilator 2506. The puncture needle 2502 can be any needle capable of providing trans-venous access (e.g., access to the femoral vein or jugular vein). In some embodiments, the puncture needle 2502 can be part of a micropuncture set. The microwire 2504 can be a short (e.g., micro) exchange wire. The microwire 2504 can have a total length of at least about 25 cm, at least about 40 cm, at least about 75 cm, at least about 100 cm, at least about 130 cm, at least about 175 cm, within a range between about 25 cm and about 175 cm, or within a range between about 40 cm and about 135 cm, e.g., depending on desired clinical performance. The microwire 2504 can advantageously be shorter than typical exchange wires used for similar procedures, thereby improving ease of use and workflow. The dilator 2506 can function to expand the size of the puncture into the blood vessel BV to facilitate insertion of additional devices (e.g., the multi-device stack 1900). The dilator 2506 can include a short interior lumen. As shown in FIG. 17D, the interior lumen of the dilator 2506 can extend from a distal opening 2506a at the distal tip of the dilator 2506 to a side port 2506b on a side of the dilator 2506. The interior lumen of the dilator 2506 can be shaped and sized to slidably receive the microwire 2504. In some embodiments, the dilator 2506 can have an angled distal tip.
FIGS. 18A-18J depict steps of an example method for inserting an intravascular device assembly (e.g., the multi-device stack 1900) into the vasculature of a patient. The method can utilize the insertion kit 2500 (e.g., one or more of the puncture needle 2502, the microwire 2504, and/or the dilator 2506). This method of insertion can be advantageous in numerous respects. In one respect, the method can enhance the procedural workflow by reducing the number of devices and swaps required for initial vascular access. For example, the method can eliminate the need for long wire exchanges. By enabling insertion of the large sheath 1902, the method can enable continual access and re-access to the treatment site. The method can be performed by a medical practitioner 2501 (e.g., a technician, a physician, a robotic surgical device, etc.). In some embodiments, the dilator 2506 may be a part of a sheath assembly with the sheath 1902.

With reference to FIG. 18A, at step 2510, initial trans-venous access can be obtained by inserting the puncture needle 2502 through the skin S of the patient and into the blood vessel BV (e.g., into the femoral vein, the jugular vein, or other blood vessel BV). In some embodiments, contrast mapping (e.g., femoral contrast mapping) can be performed if needed.

With reference to FIG. 18B, at step 2512, the microwire 2504 can be inserted through the puncture needle 2502 and into the blood vessel BV. The microwire 2504 can be long enough to extend from within the blood vessel BV and out of the skin S.

With reference to FIG. 18C, at step 2514, with microwire 2504 purchased into the blood vessel BV, the puncture needle 2502 can be removed from the patient's blood vessel BV and detached from the microwire 2504. The puncture needle 2502 can be exchanged for the dilator 2506. The dilator 2506 may be positioned within the sheath 1902 prior to the exchange (e.g., in the form of a sheath assembly) with the dilator 2506 extending distally beyond a distal end of the sheath 1902.

With reference to FIG. 18D, at step 2516, the microwire 2504 can be threaded through the dilator 2506. Specifically, the microwire 2504 can be threaded through the distal opening 2506a and out of the side port 2506b of the dilator 2506. Control of the microwire 2504 should be maintained by the medical practitioner 2501. As shown, the port 2506b can be positioned distal to the distal end of the sheath 1902.

With reference to FIG. 18E, at step 2518, the microwire 2504 can be pinned (e.g., held in place to prevent retraction or movement) and the dilator 2506 can be advanced over the microwire 2504 until the side port 2506b nears the initial puncture site (e.g., the entry site). At this step, the dilator 2506 can be stabilized in the blood vessel BV and ready for microwire 2504 removal.

With reference to FIG. 18F, at step 2520, with purchase of the dilator 2506 into the blood vessel BV, the microwire 2504 can be removed from the patient's blood vessel BV and skin S.

With reference to FIG. 18G, at step 2522, an intravascular device (e.g., the access sheath 1902 of the multi-device stack 1900) can be advanced over the dilator 2506 and into the blood vessel BV. The sheath 1902 can be inserted according to a seamless transition with no shelf.

With reference to FIG. 18H, at step 2524, with access to the blood vessel BV now established, the dilator 2506 can be removed from the patient. The dilator 2506 can be swapped for the rest of the multi-device stack 1900 (e.g., the procedure catheter 1904, the insert catheter 1906, and/or the guidewire 1908).

With reference to FIG. 18I, at step 2526, the rest of the multi-device stack 1900 (e.g., the procedure catheter 1904, the insert catheter 1906, and/or the guidewire 1908) can be inserted into the blood vessel BV through the sheath 1902. The procedure catheter 1904, the insert catheter 1906, and/or the guidewire 1908 can be advanced until the hubs of the sheath 1902 and the procedure catheter 1904 are positioned together.

With reference to FIG. 18J, at step 2528, one or more components of the multi-catheter stack can be advanced (e.g., driven) through the patient's heart H to the target location(s) in the lung L as described above with respect to FIGS. 17A-17U. The sheath 1902 can be left in the pulmonary trunk PT to provide access for device exchanges.

### Example Robotic System for a Multi-Device Stack

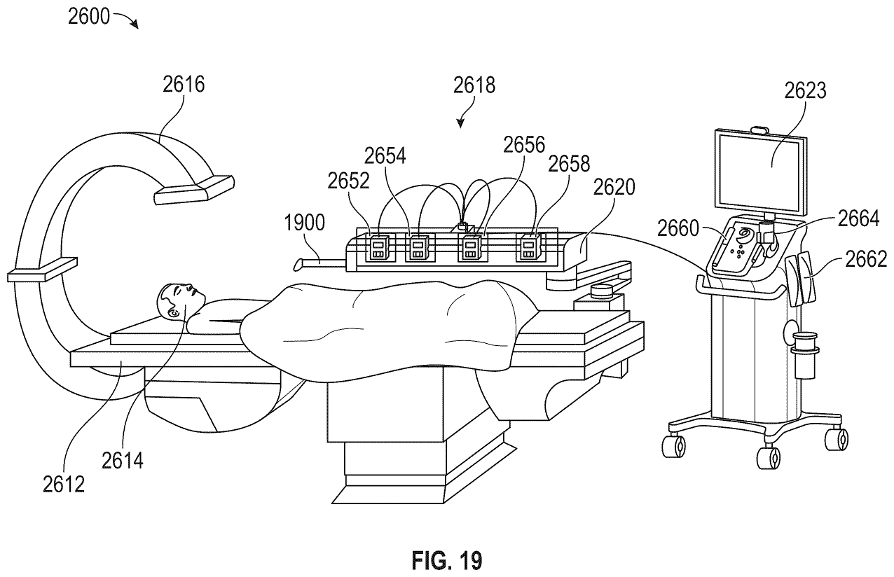

FIG. 19 depicts an example robotic system 2600 for utilizing the multi-device stack 1900. The robotic catheter system 2600 can include any of the same and/or similar features and functions as the interventional setup 10 described above with respect to FIG. 1, any of the robotic catheter systems described herein, and/or any of the robotic catheter systems described in U.S. patent application Ser. No. 18/986,519. The system 2600 can include a patient support table 2612 for supporting a patient 2614, an imaging system 2616, and a robotic interventional device drive system 2618. The drive system 2618 can include any of the same and/or similar features and functions as any of the drive systems described herein and/or any of the drive systems described in U.S. patent application Ser. No. 18/986,519. For example, any of the “hubs” described herein may be “hub assemblies” as described in U.S. patent application Ser. No. 18/986,519. The drive system 2618 can include a support table 2620, a sheath hub 2652, a procedure catheter hub 2654, an insert catheter hub 2656, and a guidewire hub 2658. The system 2600 can additionally include a display 2823. The interventional system 2600 may further include a fluidics system. The fluidics system can include a fluidics cassette 2660. The fluidics cassette 2660 can be coupled to an aspiration source, a saline source 2662, and a contrast source 2664. The fluidics cassette 2660 may include one or more fluid pathways, robotically controlled valves, and/or robotically controlled pumps or other features for providing vacuum, contrast, and/or saline (or other liquids) to one or more of the devices of the stack 1900.

The patient support table 2612, the imaging system 2616, the drive system 2618, the support table 2620, and the display 2823 can be the same as or similar to the patient support table 12, the imaging system 16, the drive system 18, the support table 2820, and the display 23 described above, respectively.

The drive system 2618 can control movement (e.g., the axial position, rotational position, and/or lateral deflection) of the multi-catheter stack. Furthermore, a fluidics management system can be integrated into the drive system to control fluid delivery to the multi-device stack 1900. Specifically, the fluidics management system can control delivery of aspiration from the cassette 2660 and/or from an aspiration source (e.g., a pump), saline from the cassette 2660 and/or from the saline source 2662, and/or contrast media from the cassette 2660 and/or from the contrast source 2664. The fluids management system can include any of the same and/or similar features and functions as the fluidics management systems described in U.S. patent application Ser. No. 18/986,519, entitled Robotic Hub Assembly, filed Dec. 18, 2024, or in U.S. patent application Ser. No. 18/666,217, entitled Fluidics Control System For Multi Catheter Stack, filed May 16, 2024, each of which is hereby expressly incorporated by reference in its entirety herein.

The sheath hub 2652 can control movement of and fluid delivery to the sheath 1902. In some embodiments, the sheath hub 2652 can be fluidically coupled to the cassette 2660, the aspiration source, the saline source 2662, and/or the contrast source 2664 to deliver aspiration, saline, and/or contrast media through the sheath 1902. The procedure catheter hub 2654 can control movement of and fluid delivery to the procedure catheter 1904. In some embodiments, the procedure catheter hub 2654 can be fluidically coupled to the cassette 2660, the aspiration source, the saline source 2662, and/or the contrast source 2664 to deliver aspiration, saline, and/or contrast media through the procedure catheter 1904. The insert catheter hub 2656 can control movement of and fluid delivery to the insert catheter 1906. In some embodiments, the insert catheter hub 2656 can be fluidically coupled to the cassette 2660, the saline source 2662 and/or the contrast source 2664 to deliver saline and/or contrast media through the insert catheter 1906. The guidewire hub 2658 can control movement of and fluid delivery to the guidewire 1908. In some embodiments, the guidewire hub 2658 can be fluidically coupled to the cassette 2660, the aspiration source, and/or the saline source 2662 to deliver aspiration and/or saline through the guidewire 1908. In some embodiments, the guidewire hub 2658 can be fluidically coupled to a drug source (e.g., a source of tPA) to deliver drugs (e.g., tPA) through the guidewire 1908.

Additional details regarding robotic systems that may be used to operate multi-catheter assemblies can be found in U.S. patent application Ser. No. 18/784,630, entitled System For Remote Medical Procedure, filed Jul. 25, 2024, which is hereby expressly incorporated by reference in its entirety herein.

### Example Access Dilator With a Pigtail Tip

The present disclosure provides an access dilator and a multi-device stack for treatment of pulmonary embolism (PE). The systems and methods disclosed herein can decrease transfer time, increase access, and improve the timeliness of therapy, thereby reducing the PE mortality rate. Furthermore, the systems and methods described herein can reduce the total number of devices and device swaps required for PE treatment. The systems and methods described herein can also be used for other interventional procedures, including thrombectomy procedures in any other portion of the vascular system.

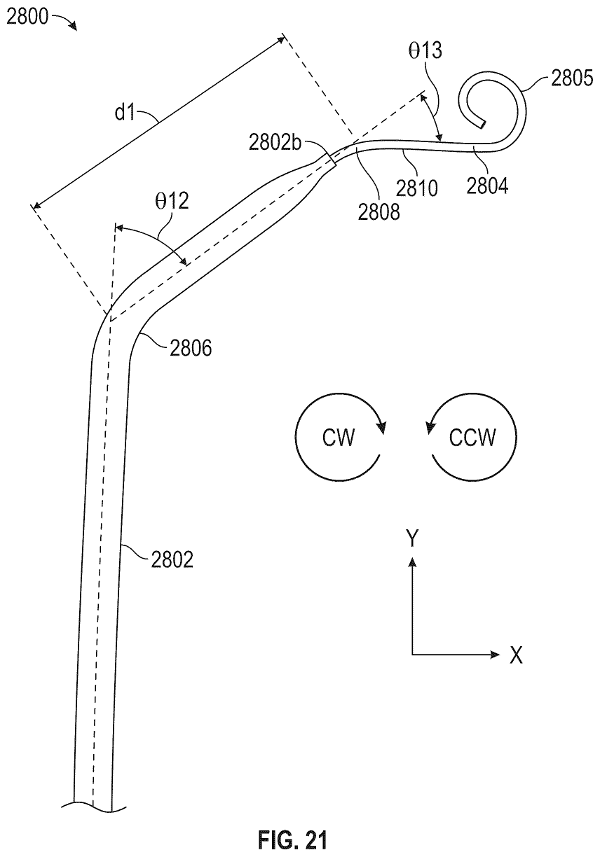

FIGS. 21-24B depict various embodiments and configurations of an access dilator (e.g., access dilator 2800, 3200, 3300). Any one or more of the access dilators 2800, 3200, 3300 can be included as an additional or alternative device of the multi-device stack 1900 described above. For example, any one or more of the access dilators 2800, 3200, 3300 can be an example of the insert catheter 1906 described above and/or utilized in the same or similar manner as the insert catheter 1906 described above and vice versa. The access dilator can be utilized during a thrombectomy procedure to obtain access to a treatment site within a patient's vasculature. For example, the access dilator can be used during treatment of pulmonary embolism to obtain access to the patient's pulmonary arteries. The access dilator can be initially inserted into a patient's vasculature via a puncture into a vein (e.g., the femoral vein or jugular vein). The access dilator may be inserted through another device that has already been introduced into the vein entry point. In some embodiments, advantageously, without utilizing a pre-inserted guidewire, the access dilator can then be advanced through the patient's vasculature to the target treatment site. The access dilator can extend/advance as a rail from a catheter (e.g., an aspiration catheter). The access dilator can include a pigtail tip. The pigtail tip can advantageously enable atraumatic traversal of the access dilator through the complex curvature and valve structures of the heart and into the pulmonary trunk. For example, the access dilator can atraumatically navigate from the entry point into the right atrium, through the tricuspid valve, and through the right ventricle up to the pulmonary valve and pulmonary trunk. The pigtail tip of the access dilator can be maintained in the curled configuration while traversing through the tortuous anatomy of the heart. After the access dilator establishes access to the treatment site, additional instruments (e.g., a procedure catheter, aspiration catheter or other large bore catheter) can be advanced via the access dilator to the treatment site. For example, the access dilator may act as a rail over which additional instruments may advance. Accordingly, the access dilator can advantageously facilitate delivery of one or more procedural devices (e.g., an aspiration catheter) to the treatment site.

FIG. 21 depicts a side view of an embodiment of an access dilator 2800. The access dilator 2800 can be used to provide access to a treatment site (e.g., within a pulmonary artery) of a patient for delivery of one or more procedural devices (e.g., an aspiration catheter). As shown in FIG. 21, the access dilator 2800 can include an obturator portion 2802 and a pigtail portion 2804. The obturator portion 2802 can include and extend between a proximal end (not shown) and a distal end 2802b. The distal end 2802b of the obturator portion 2802 can be tapered in a distal direction. The obturator portion 2802 can include a lumen 3212 for slidably receiving a device (e.g., a guidewire, a stiffening member, and/or the pigtail portion 2804). The obturator portion 2802 can include a larger outer diameter than the pigtail portion 2804. The outer diameter of the distal end 2802b can decrease distally to the pigtail portion 2804 to form the tapered or conical distal end 2802b of the obturator portion 2802. The obturator portion 2802 can form a long rail with a length sufficient to reach treatment sites deep within a patient's vasculature. The obturator portion 2802 can have a substantially annular or substantially circular cross-section.

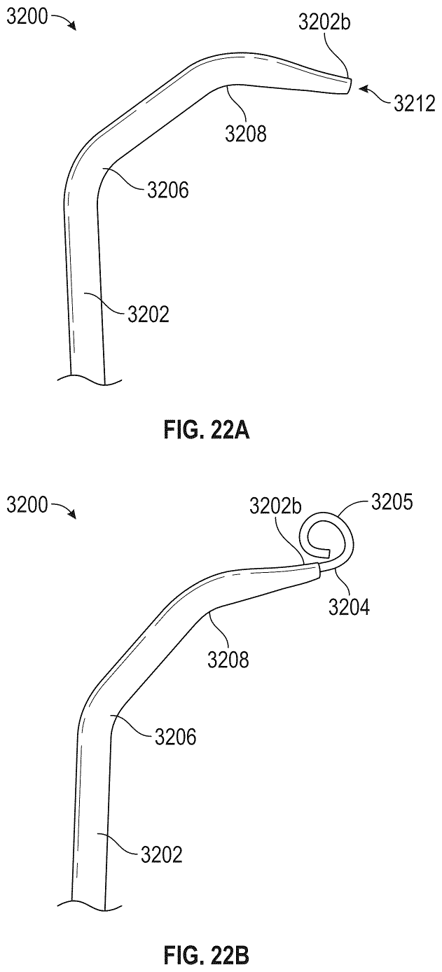

With continued reference to FIG. 21, the pigtail portion 2804 can be coupled to and/or extend from the obturator portion 2802. The pigtail portion 2804 can extend distally from the distal end 2802b of the obturator portion 2802. The pigtail portion 2804 can include a pigtail tip 2805. As shown in FIG. 21, the pigtail tip 2805 can be shaped as a spiral or curl. In some embodiments, the pigtail portion 2804 can be a fixed and/or integral part of the obturator portion 2802 (e.g., such that it cannot be advanced or retracted relative to the obturator portion 2802). In other embodiments, the pigtail portion 2804 may be movable relative to and/or formed separately from the obturator portion 2802 (e.g., the pigtail portion 2804 can be a movable pigtail tipped catheter which can be rotated, advanced, retracted, or removed completely from the lumen of the obturator portion 2802) (see FIGS. 22A-22B). In some embodiments, the pigtail portion 2804 can include a lumen. The lumen can extend through the pigtail portion 2804 to the distal end of the pigtail tip 2805. In some embodiments, the lumen of the pigtail portion 2804 can be in fluid communication with the central lumen of the obturator portion 2802. In some embodiments, the pigtail portion 2804 can include one or more side ports 2810. The one or more side ports 2810 can be in fluid communication with the lumen of the pigtail portion 2804 and/or the lumen 3212 of the obturator portion 2802. The one or more side ports 2810 can be used to inject contrast media. In some embodiments, one or more side ports 2810 can be located on the obturator portion 2802.

With continued reference to FIG. 21, the access dilator 2800 can include a first bend 2806 and a second bend 2808. The first bend 2806 can be formed in the obturator portion 2802. In some embodiments, the first bend 2806 can have a first angle θ12 of between 10 degrees and 70 degrees, between about 10 degrees and about 70 degrees, between 20 degrees and 60 degrees, or between about 20 degrees and about 60 degrees (e.g., 45 degrees or about 45 degrees), depending upon desired clinical performance. The second bend 2808 can be located at a position distal to the first bend 2806. In some embodiments, the second bend 2808 can be positioned away from the first bend 2806 by a distance d10 between 1 cm and 6 cm, between about 1 cm and about 6 cm, between 2 cm and 5 cm, or between about 2 cm and about 5 cm. In some embodiments, the second bend 2808 can be formed in the pigtail portion 2804. As shown in FIG. 21, in some embodiments, the second bend 2808 can be formed at the junction between the distal end 2802b of the obturator portion 2802 and the proximal most portion of the pigtail portion 2804 that extends from the obturator portion 2802. In other embodiments, the second bend 2808 can be formed in the obturator portion 2802 (see FIGS. 22A-22B). In some embodiments, the second bend 2808 can have a second angle θ13 of between 10 degrees and 70 degrees, between about 10 degrees and about 70 degrees, between 20 degrees and 60 degrees, or between about 20 degrees and about 60 degrees (e.g., 45 degrees or about 45 degrees), depending upon desired clinical performance. As shown in FIG. 21, the portion of the access dilator 2800 proximal to the first bend 2806, the portion of the access dilator 2800 between the first bend 2806 and the second bend 2808, and the portion of the access dilator 2800 between the second bend 2808 and the pigtail tip 2805 can each be substantially straight. In other embodiments, one or more of those portions can be curved.

With continued reference to FIG. 21, the first bend 2806 and the second bend 2808 can both be oriented in substantially the same direction. For example, the first bend 2806 and the second bend 2808 can both be oriented in a clockwise direction CW (see FIG. 21) or both be oriented in a counterclockwise direction CCW. For example, the first bend 2806 can bend in a CW direction relative to a central axis of the obturator portion 2802 proximal to the first bend 2806 and the second bend 2808 can bend in a CW direction relative to a central axis extending through the access dilator 2800 between the first bend 2806 and the second bend 2808. In some embodiments, the pigtail tip 2805 of the pigtail portion 2804 can curl in the opposite direction to the direction of the first bend 2806 and the second bend 2808. For example, in the embodiments shown in FIG. 21, the pigtail tip 2805 curls in the counterclockwise direction CCW (e.g., relative to a central axis of the access dilator 2800 between the second bend 2808 and the pigtail tip 2805), and the first bend 2806 and the second bend 2808 are oriented in the clockwise direction CW. In other embodiments, the pigtail tip 2805 can curl in the same direction as the first bend 2806 and the second bend 2808.

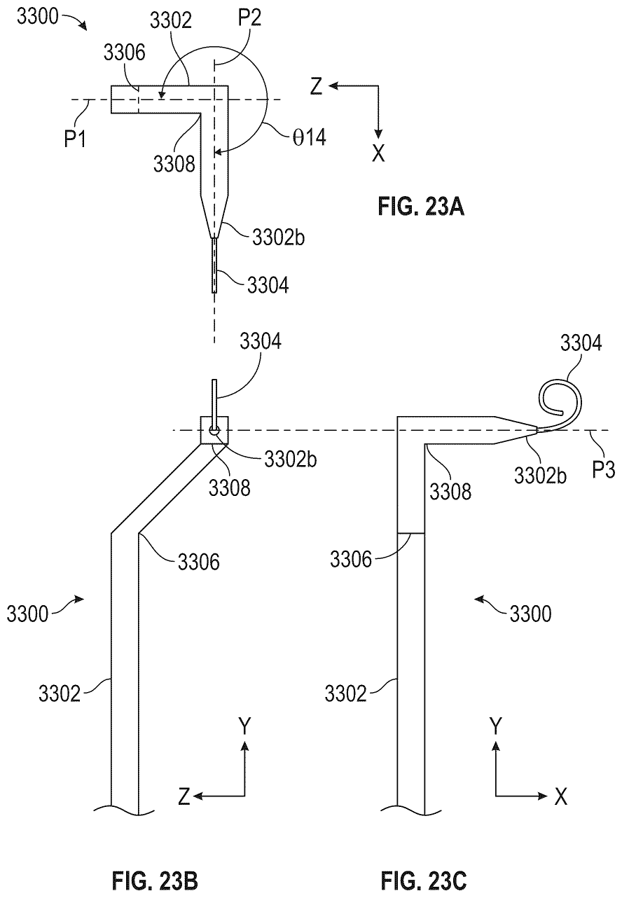

As shown in FIG. 21, the first bend 2806 and the second bend 2808 can both be oriented within substantially the same plane (e.g., the X-Y plane illustrated in FIG. 21). Additionally, the pigtail tip 2805 can curl within the same plane as the first bend 2806 and the second bend 2808. For example, as shown in FIG. 21, the first bend 2806, the second bend 2808, and the curl of the pigtail tip 2805 can be oriented within the same plane such that the access dilator 2800 can lay flat or substantially flat on a flat surface (e.g., the access dilator 2800 can have a planar or substantially planar shape that lies within the X-Y plane illustrated in FIG. 21). In other embodiments, one or more of the first bend 2806, the second bend 2808, and the pigtail portion 2804 can be oriented with a different plane (see FIGS. 23A-23B).

The access dilator 2800, including the obturator portion 2802 and/or the pigtail portion 2804, can be flexible and/or resilient. In some embodiments, the pigtail portion 2804 can be made from a softer and/or more flexible material than the obturator portion 2802. The pigtail portion 2804 can be softer to provide an atraumatic distal end, whereas the obturator portion 2802 can be more rigid to facilitate navigation and push-ability. In some embodiments, the access dilator 2800 can include and/or be used with a stiffening member (e.g., a guidewire) to selectively straighten the bends (e.g., the first bend 2806 and/or the second bend 2808) and/or the pigtail tip 2805 of the access dilator 2800. The access dilator 2800 can include an interior lumen extending through the obturator portion 2802 and/or the pigtail portion 2804 to allow for delivery of a guidewire, catheter, or other instrument through the access dilator 2800. The access dilator 2800 can be manufactured as a single piece or multiple pieces.

FIGS. 22A-22B depict another embodiment of an access dilator 3200. The access dilator 3200 can be used in the same or similar manner as and can incorporate the same or similar components or features as the other access dilators 2800, 3300 described herein, and may include additional components or features as described further herein. Thus, reference numerals used to designate the various features or components of the access dilator 3200 are identical to those used for identifying the corresponding features of components of the access dilators 2800, 3300, except that the reference numerals have been incremented to begin with “32” instead of “28” or “33”. Therefore, the structure and description for the various features of the access dilators 2800, 3300 and how they are operated are understood to also apply to the corresponding features of the access dilator 3200, except or in addition to as described below.

As shown in FIGS. 22A-22B, the access dilator 3200 can include an obturator portion 3202 and a pigtail portion 3204 that is formed separately from the obturator portion 3202. The pigtail portion 3204 can include pigtail tipped catheter that can be rotated, advanced, retracted, or removed completely from the lumen 3212 of the obturator portion 3202.

FIG. 22A depicts a side view of the access dilator 3200 with the pigtail portion 3204 in a first configuration (e.g., a retracted or removed configuration). In the retracted configuration, the pigtail portion 3204 can be retracted within the lumen 3212 of the obturator portion 3202 such that the pigtail portion 3204 does not extend from the distal end 2802b of the obturator portion 3202. The pigtail portion 3204 may alternatively be completely removed from the lumen 3212 of the obturator portion 3202. In such embodiments, the pigtail portion 3204 may be part of or coupled to a separate wire, catheter, or other device that can be advanced within the lumen 3212. As described herein, the access dilators may be coupled to a robotic drive table and advanced within the vasculature. For example, an access dilator may be coupled to a hub and/or hub adapter. In embodiments in which the pigtail portion is part of or coupled to a separate wire, catheter, or other device, the separate wire, catheter, or other device may be coupled to its own hub and/or hub adapter, while the remaining portion of the dilator 3200 (e.g., obturator 3202) may be coupled to its own hub and/or hub adapter.

Depending on the desired clinical performance or clinical application, the access dilator 3200 can be advanced with the pigtail portion 3204 in a retracted or removed configuration through specific portions of vasculature that are less tortuous or less prone to injury (e.g., from an entry point in the femoral artery up to the right atrium). As shown in FIG. 22A, the obturator portion 3202 can include a first bend 3206, a second bend 3208, a tapered distal end 3202b, and/or a lumen 3212. The second bend 3208 can be formed in the obturator portion 3202 at a location that is proximal to the distal end 3202b. The second bend 3208 can be proximal to the start of tapering at the distal end 3202b.

FIG. 22B depicts a side view of the access dilator 3200 with the pigtail portion 3204 in a second configuration (e.g., a deployed configuration). In the deployed configuration, the pigtail portion 3204 can extend at least partially out of the distal end 3202b of the obturator portion 3202 to define a curled pigtail tip 3205. The pigtail portion 3204 can be moved to the deployed configuration by advancing the pigtail portion 3204 through the lumen 3212 of the obturator portion 3202 until the pigtail tip 3205 extends at least partially out from the distal end 3202b of the obturator portion 3202. Depending on the desired clinical performance or clinical application, the access dilator 3200 can be advanced with the pigtail portion 3204 in the deployed configuration through specific portions of vasculature that are tortuous and/or prone to injury (e.g., through complex curvature and valve structures of the heart and into the pulmonary trunk). For example, with the pigtail portion 3204 in the deployed configuration, the access dilator 3200 can atraumatically navigate from the entry point into the right atrium, through the tricuspid valve, and through the right ventricle up to the pulmonary valve and pulmonary trunk. In some embodiments, the pigtail portion 3204 can include an interior lumen. In other embodiments, the pigtail portion 3204 may not include an interior lumen. In some embodiments, multiple pigtail portions 3204 may be used with a single obturator portion 3202 (e.g., pigtail portions 3204 having different orientations of the pigtail tip 3205). In some embodiments, a pigtail portion 3204 that may be moved relative to the obturator portion 3202 may be rotated relative to the obturator portion 3202 so that the pigtail tip 3205 can curve in different directions (e.g., CW or CCW) relative to the obturator portion 3202.

FIGS. 23A-23C depict schematic views of another embodiment of an access dilator 3300. The access dilator 3300 can be used in the same or similar manner as and can incorporate the same or similar components or features as the other access dilators described herein, and may include additional components or features as described further herein. Thus, reference numerals used to designate the various features or components of the access dilators 2800, 3200 are identical to those used for identifying the corresponding features of components of the access dilator 3300, except that the reference numerals have been incremented to being with “33” instead of “28” or “32”. Therefore, the structure and description for the various features of the access dilators 2800, 3200 and how they are operated are understood to also apply to the corresponding features of the access dilator 3300, except or in addition to as described below.

FIG. 23A depicts a top view of the access dilator 3300, FIG. 23B depicts a front view of the access dilator 3300, and FIG. 23C depicts a side view of the access dilator 3300. As shown in FIGS. 23A-23C, the access dilator 3300 can include an obturator portion 3302, a pigtail portion 3304, a first bend 3306, and a second bend 3308. The obturator portion 3302 can include a distal end 3302b. As shown in FIGS. 23A-23C, the first bend 3306 and the second bend 3308 can be oriented in different planes. With reference to FIGS. 3A and 3B, the first bend 3306 can be oriented within a first plane P1 that is parallel to the Z-Y plane. With reference to FIGS. 3A and 3C, the second bend 3308 can be oriented within a second plane P2 that is parallel to the X-Y plane. As shown in FIG. 23C, in some embodiments, the pigtail portion 3304 can curl within the same plane as the second bend 3308. With reference to FIG. 23A, the second plane P2 can be angled relative to the first plane P1 by a third angle θ₃. The third angle θ₃ can be any value between 35 degrees and 325 degrees, between about 35 degrees and about 325 degrees, between 45 degrees and 315 degrees, between about 45 degrees and about 315 degrees (e.g., 90 degrees, about 90 degrees, 135 degrees, about 135 degrees, 225 degrees, about 225 degrees, 270 degrees, or about 270 degrees). FIG. 23A shows the second plane P2 angled relative to the first plane P1 by about 270 degrees. In some embodiments, having the first bend 3306 and second bend 3308 in different planes can advantageously enhance navigation of the access dilator 3300 through tortuous vasculature. Specifically, the orientation and angle of the first bend 3306 and the second bend 3308 can be designed to accommodate and/or replicate specific turns and/or portions of vasculature to facilitate navigation therethrough.

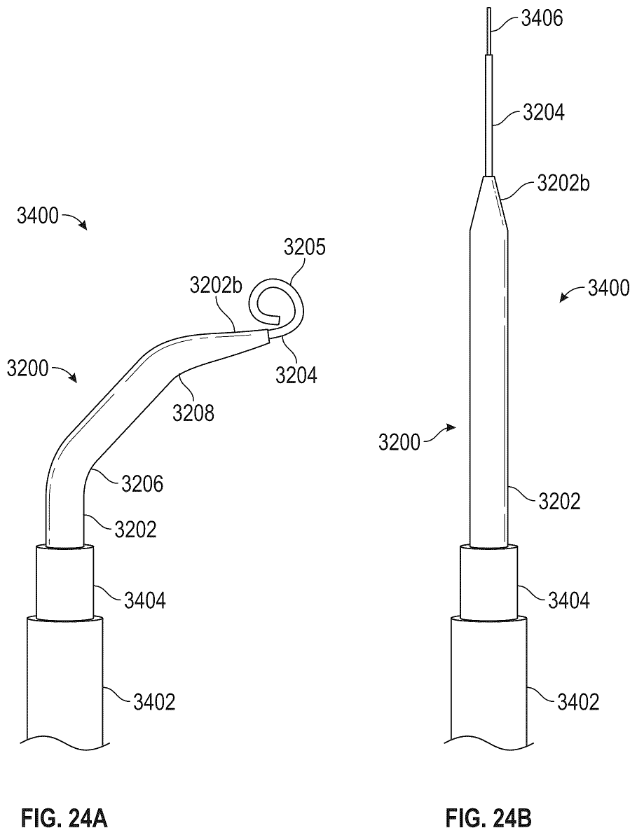

FIGS. 24A-31B depict schematic views of a multi-device stack including the access dilator 3200. The multi-device stack 3400 can include a sheath 3402 (e.g., a guide catheter), a procedure catheter 3404 (e.g., an aspiration catheter), an access dilator (e.g., access dilator 2800, 3200, 3300), and/or a stiffening member, such as a guidewire 3406. The multi-device stack 3400 may be manually driven or robotically driven to perform a vascular procedure (e.g., a PE procedure). The devices of the multi-device stack may be coupled to corresponding hubs and robotically controlled as described herein.

In some embodiments, the sheath 3402 may include any of the same or similar features and/or functions as the sheath 1902 and vice versa. In some embodiments, the procedure catheter 3404 may include any of the same and/or similar features and/or functions as the procedure catheter 1904 and vice versa. In some embodiments, the guidewire 3406 may include any of the same and/or similar features and/or functions as the guidewire 1908.

FIGS. 24A-24B illustrate the multi-device stack 3400 with the access dilator 3200, but the access dilator 2800 or access dilator 3300 could alternatively be used with the multi-device stack 3400. In some cases, the access dilator 3200 may be advanced as part of the multi-device stack 3400 to the treatment site. For example, the access dilator 3200 may be positioned within the procedure catheter 3404 and/or the sheath 3402. The access dilator 3200 can be positioned coaxially within the procedure catheter 3404. The procedure catheter 3404 can be positioned coaxially within the sheath 3402. The guidewire 3406 may be positioned within the lumen 3212 of the access dilator 3200. The guidewire 3406 may extend out of the distal end 3202b of the access dilator 3200. The multi-device stack 3400 may be advanced to the treatment site with the pigtail tip 3205 of the access dilator 3200 leading the multi-device stack 3400 to provide for atraumatic navigation. The guidewire 3406 can include a stiffening member or stylet for straightening the pigtail tip 3205.

FIG. 24A shows the multi-device stack 3400 with the access dilator 3200 in a deployed configuration. When in the deployed configuration, one or more bends (e.g., the first bend 3206 and/or the second bend 3208) can be formed in the access dilator 3200 and the pigtail tip 3205 can be curled. The obturator portion 3202 and/or the pigtail portion 3204 can be pre-formed to have the one or more bends and the curl of the pigtail tip 3205 when in a relaxed or default state. Accordingly, the access dilator 3200 can be biased to the deployed configuration when the guidewire 3406 is removed or retracted from the access dilator 3200. The access dilator 3200 may be maintained in the deployed configuration while the multi-device stack 3400 and/or the access dilator 3200 itself is advanced through a patient's vasculature.

FIG. 24B shows the multi-device stack 3400 with the access dilator 3200 in a straightened configuration. When in the straightened configuration, one or more of the first bend 3206, the second bend 3208, and the pigtail tip 3205 can be straightened. The access dilator 3200 can be transitioned to the straightened configuration by advancing the guidewire 3406 within the lumen 3212 of the obturator portion 3202 and/or the pigtail portion 3204 to straighten one or more of the first bend 3206, the second bend 3208, and the pigtail tip 3205. The access dilator 3200 may be maintained in the straightened configuration to facilitate insertion of the access dilator 3200 into the multi-device stack 3400 (e.g., into a proximal end of the sheath 3402 and/or the procedure catheter 3404) or to facilitate traversal through portions of vasculature. Retraction of the guidewire 3406 from the access dilator 3200 can transition the access dilator 3200 back to the deployed configuration. In other embodiments, the access dilator 3200 may be pre-formed to a straightened shape when in the relaxed state, and the guidewire 3406 can be inserted to define one or more of the first bend 3206, the second bend 3208, and the pigtail tip 3205. In other embodiments, other mechanisms can be used to transition the access dilator 3200 between deployed and straightened configurations including but not limited to balloon catheter systems, magnetic systems, motorized systems, and shape memory materials (e.g., nitinol).

### Example Method of Operating the Access Dilator and/or the Multi-Device Stack

A pulmonary embolism can begin as a thrombus in the patient's femoral vein. An embolus can break off from the thrombus and travel through the patient's vasculature until it becomes lodged in the patient's pulmonary arteries, thereby creating a pulmonary embolism. In such a situation, a medical intervention utilizing the multi-device stack 3400 and/or access dilator 2800, 3200, 3300 can be undertaken to treat the pulmonary embolism. As mentioned above, the multi-device stack 3400 and/or access dilator 2800, 3200, 3300 can advantageously extend from an entry point in the patient's skin to a treatment site in the patient's pulmonary trunk or pulmonary arteries. The multi-device stack 3400 and/or access dilator 2800, 3200, 3300 can accordingly provide direct access to the treatment site. In some embodiments, the multi-device stack 3400 and/or access dilator 2800, 3200, 3300 can initially be introduced into the patient's vasculature at an entry point into the patient's femoral vein. From the entry point in the femoral vein, the multi-device stack 3400 and/or access dilator 2800, 3200, 3300 can be advanced into the inferior vena cava. After climbing up the inferior vena cava, the multi-device stack 3400 and/or access dilator 2800, 3200, 3300 can be advanced into the right atrium of the heart. From the right atrium, the multi-device stack 3400 and/or access dilator 2800, 3200, 3300 can be advanced through the tricuspid valve and into the right ventricle. From the right ventricle, the multi-device stack 3400 and/or access dilator 2800, 3200, 3300 can be advanced through the pulmonary valve and into the pulmonary trunk. From the pulmonary trunk, one or more components of the multi-device stack 3400 and/or access dilator 2800, 3200, 3300 can be advanced into either the right lung or the left lung, depending on the location of the pulmonary embolism. Within either the right lung or the left lung, the multi-device stack 3400 and/or access dilator 2800, 3200, 3300 can be advanced into a pulmonary artery and to a treatment site within the pulmonary artery. The treatment site can be at and/or adjacent to the location of the pulmonary embolism. In some embodiments, the pigtail portion 2804, 3204, 3304 (e.g., the pigtail tip 2805, 3205 of the access dilator 2800, 3200, 3300) can be positioned adjacent to the treatment site. In some embodiments, the procedure catheter 3404 (e.g., an aspiration catheter) can be advanced via the access dilator 2800, 3200, or 3300 to the treatment site (e.g., the access dilator 2800, 3200, or 3300 can function as a rail to advance the procedure catheter 3404). For example, the procedure catheter 3404 can be advanced via the access dilator 2800, 3200, 3300 in a similar manner as described with respect to the procedure catheter 1904 relative to the insert catheter 1906. The sheath 3402 may be advanced relative to the access dilator 2800, 3200, or 3300 in a similar manner as described with respect to the sheath 1902 relative to the insert catheter 1906. The guidewire 3406 may be advanced relative to the access dilator 2800, 3200, or 3300 in a similar manner as described with respect to the guidewire 1908 and vice versa.

Once at the treatment site, the aspiration can be applied via the procedure catheter 3404 to aspirate and remove at least a portion of the pulmonary embolism. In some embodiments, the multi-device stack 3400 and/or access dilator 2800, 3200, 3300 can alternatively be introduced into the patient's vascular system through an entry point into the jugular vein. From the entry point in the jugular vein, the multi-device stack 3400 and/or access dilator 2800, 3200, 3300 can be advanced down into the right atrium. From the right atrium, the multi-device stack 3400 and/or access dilator 2800, 3200, 3300 can be advanced to the pulmonary artery in the same manner as described above.

Various systems and methods are described herein primarily in the context of a neurovascular access or procedure. However, the inventors contemplate applicability of the disclosed catheters, systems, and methods to any of a wide variety of alternative applications, including within the coronary vascular or peripheral vascular systems as well as other hollow organs or tubular structures in the body.

While the foregoing describes robotically driven interventional devices and manually driven interventional devices, the devices may be manually driven, robotically driven, or any combination of manually and robotically driven interventional devices, as will be appreciated by those of skill in the art in view of the disclosure herein. For example, in certain embodiments, a hub may be removed from a mount to allow the hub and its corresponding interventional device to be manually driven. Additional details regarding procedures including combinations of manual and robotically driven devices are provided in U.S. application Ser. No. 18/545,687, titled SYSTEM WITH REMOVABLE HUBS FOR MANUAL AND ROBOTIC PROCEDURE, filed Dec. 19, 2023, the entirety of which is hereby incorporated by reference herein

While certain arrangements of the inventions have been described, these arrangements have been presented by way of example only, and are not intended to limit the scope of the disclosure. Indeed, the novel methods and systems described herein may be embodied in a variety of other forms. Furthermore, various omissions, substitutions and changes in the systems and methods described herein may be made without departing from the spirit of the disclosure. The accompanying claims and their equivalents are intended to cover such forms or modifications as would fall within the scope and spirit of the disclosure. Accordingly, the scope of the present inventions is defined only by reference to the appended claims.

Features, materials, characteristics, or groups described in conjunction with a particular aspect, arrangement, or example are to be understood to be applicable to any other aspect, arrangement or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing arrangements. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a subcombination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some arrangements, the actual steps taken in the processes illustrated and/or disclosed may differ from those shown in the figures. Depending on the arrangement, certain of the steps described above may be removed, others may be added. Furthermore, the features and attributes of the specific arrangements disclosed above may be combined in different ways to form additional arrangements, all of which fall within the scope of the present disclosure. Also, the separation of various system components in the implementations described above should not be understood as requiring such separation in all implementations, and it should be understood that the described components and systems can generally be integrated together in a single product or packaged into multiple products.

For purposes of this disclosure, certain aspects, advantages, and novel features are described herein. Not necessarily all such advantages may be achieved in accordance with any particular arrangement. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain arrangements include, while other arrangements do not include, certain features, elements, and/or steps. Thus, such conditional language is not generally intended to imply that features, elements, and/or steps are in any way required for one or more arrangements or that one or more arrangements necessarily include logic for deciding, with or without user input or prompting, whether these features, elements, and/or steps are included or are to be performed in any particular arrangement.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain arrangements require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain arrangements, the terms “generally parallel” and “substantially parallel” refer to a value, amount, or characteristic that departs from exactly parallel by less than or equal to 15°, 10°, 5°, 3°, 1 degree, or 0.1 degree. The ranges disclosed herein also encompass any and all overlap, sub-ranges, and combinations thereof, and any specific values within those ranges. Language such as “up to,” “at least,” “greater than,” “less than,” “between,” and the like includes the number recited. Numbers and values used herein preceded by a term such as “about” or “approximately” include the recited numbers. For example, “approximately 7 mm” includes “7 mm” and numbers and ranges preceded by a term such as “about” or “approximately” should be interpreted as disclosing numbers and ranges with or without such a term in front of the number or value such that this application supports claiming the numbers, values and ranges disclosed in the specification and/or claims with or without the term such as “about” or “approximately” before such numbers, values or ranges such, for example, that “approximately two times to approximately five times” also includes the disclosure of the range of “two times to five times.” The scope of the present disclosure is not intended to be limited by the specific disclosures of preferred arrangements in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

What is claimed is:

1. A system for treating a pulmonary embolism, the system comprising:
a coaxial multi-device assembly comprising:
a sheath coupled to a sheath hub, wherein the sheath hub is configured to adjust an axial position of the sheath;
a procedure catheter positioned within the sheath and coupled to a procedure catheter hub, wherein the procedure catheter hub is configured to adjust an axial position of the procedure catheter; and
an access device positioned within the procedure catheter and coupled to an access device hub, wherein the access device hub is configured to adjust an axial position and a rotational position of the access device, wherein the access device comprises a tip configured to transition between a curled configuration and a straight configuration, and
a robotic drive system configured to drive axial movement of the sheath hub, the procedure catheter hub, and the access device hub.

2. The system of claim 1, wherein the coaxial multi-device assembly further comprises a guidewire positioned within the access device and coupled to a guidewire hub, wherein the guidewire hub is configured to drive axial and rotational movement of the guidewire.

3. The system of claim 2, wherein the access device is in fluid communication with a contrast source and configured to inject contrast while the guidewire is positioned within the access device.

4. The system of claim 2, wherein the guidewire comprises a hollow guidewire.

5. The system of claim 2, wherein the guidewire is in fluid communication with a source of a drug to provide the drug through the guidewire.

6. The system of claim 5, wherein the drug comprises a tissue plasminogen activator.

7. The system of claim 2, wherein the guidewire is in fluid communication with an aspiration source to provide aspiration through the guidewire.

8. The system of claims 2, wherein the guidewire comprises a tip configured to transition between a curled configuration and a straight configuration.

9. The system of claim 1, wherein the access device comprises a tapered distal section.

10. The system of claim 1, wherein the robotic drive system comprises:
a sheath hub adapter configured to couple to the sheath hub to drive axial movement of the sheath hub;
a procedure catheter hub adapter configured to couple to the procedure catheter hub to drive axial movement of the procedure catheter; and
an access device hub adapter configured to couple to the access device hub to drive axial movement of the access device.

11. The system of claim 10, wherein the sheath hub adapter is magnetically coupled to the sheath hub, wherein the procedure catheter hub adapter is magnetically coupled to the procedure catheter hub, and wherein the access device hub adapter is magnetically coupled to the access device hub.

12. The system of claim 1, wherein the access device is an access catheter or an access dilator.

13. A method of treating a pulmonary embolism, comprising:
introducing a multi-device assembly into a blood vessel of a patient, the multi-device assembly comprising a sheath, a procedure catheter, and an access device;
advancing the multi-device assembly through an inferior vena cava and into a right atrium of the patient;
advancing the access device through the right atrium of the patient, through a right ventricle of the patient, and into a pulmonary trunk of the patient;
advancing the access device into a pulmonary branch and to a treatment site;
advancing the procedure catheter over the access device and to the treatment site;
removing the access device; and
performing a thrombectomy using the procedure catheter.

14. The method of claim 13, further comprising advancing the sheath over the procedure catheter and the access device into the pulmonary trunk.

15. The method of claim 14, wherein the step of advancing the sheath over the procedure catheter and the access device into the pulmonary trunk is performed after advancing the access device into the pulmonary trunk of the patient and before advancing the access device into the pulmonary branch.

16. The method of claim 14, wherein the multi-device assembly further comprises a guidewire, wherein the step of advancing the access device through the right atrium of the patient, through the right ventricle of the patient, and into the pulmonary trunk of the patient comprises advancing the access device and the guidewire through the right atrium of the patient, through the right ventricle of the patient, and into the pulmonary trunk of the patient, wherein the step of advancing the access device into the pulmonary branch and to the treatment site comprises advancing the access device and the guidewire into the pulmonary branch and to the treatment site, and wherein the step of removing the access device comprises removing the access device and the guidewire.

17. The method of claim 16, further comprising, after performing the thrombectomy with the procedure catheter, withdrawing the sheath and the procedure catheter from the pulmonary branch and advancing the sheath and the procedure catheter into a different pulmonary branch without reinserting the access device and the guidewire.

18. The method of claim 16, wherein the guidewire comprises a hollow guidewire, the method further comprising aspirating through the guidewire to anchor a blood clot to the guidewire.

19. The method of claim 16, wherein the guidewire comprises a tip configured to transition between a curled configuration and a straight configuration, wherein advancing the guidewire through the right atrium of the patient, through the right ventricle of the patient, and into the pulmonary trunk of the patient is performed while the tip of the guidewire is in the curled configuration.

20. The method of claim 13, wherein the access device comprises a tip configured to transition between a curled configuration and a straight configuration, wherein advancing the access device through the right atrium of the patient, through the right ventricle of the patient, and into the pulmonary trunk of the patient is performed while the tip of the access device is in the curled configuration.
